(19) United States
(12) Patent Application Publication
(10) Pub. No.: US 2023/0132386 A9
(43) Pub. Date: Apr. 27, 2023

(54) GRANZYME B DIRECTED IMAGING AND THERAPY

(71) Applicant: CytoSite Biopharma Inc., Sudbury, MA (US)

(72) Inventors: Geoffrey Malcolm Bilcer, Shorewood, MN (US); Terence Alfred Kelly, Ridgefield, CT (US)

(21) Appl. No.: 17/430,180

(22) PCT Filed: Feb. 12, 2020
(86) PCT No.: PCT/US2020/017980

§ 371 Date: Aug. 11, 2021

Related U.S. Application Data
(60) Provisional application No. 62/804,893, filed on Feb. 13, 2019.

(51) Int. Cl. C07D 519/00; C07D 487/06; A61B 6/03

(52) U.S. Cl. CPC C07D 519/00; C07D 487/06; A61B 6/037

(57) ABSTRACT

Provided herein are compounds of Formula (I) below that are useful for imaging Granzyme B. Methods of imaging Granzyme B and treating an immunoregulatory abnormality, pharmaceutical compositions, and kits comprising the Granzyme B imaging agents are also provided.

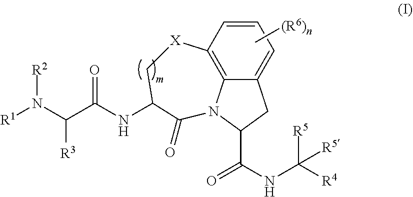

### TECHNICAL FIELD

This disclosure relates to compounds useful for imaging techniques, and more particularly to compounds that are useful for imaging Granzyme B using medical imaging, including positron emission tomography.

### BACKGROUND

Granzyme B is a serine-protease released through exocytosis by cytotoxic lymphocytes (CTL) during the cellular immune response, and represents one of the two dominant mechanisms, along with the FAS/FASL pathway, by which T-cells mediate cancer-cell death. Granzyme B is released along with the pore-forming protein perforin at the immunological-synapse formed between T-cells and their targets. A portion of the released Granzyme B then enters cancer cells, primarily through perforin-pores, where it activates multiple substrates leading to activation of the caspase cascade.

### SUMMARY

The present application provides heterocyclic compounds of Formula I below and their use as imaging agents or therapy both related to Granzyme B.

In one aspect, this disclosure relates to a compound of Formula I or a pharmaceutically acceptable salt thereof, or a stereoisomer or tautomer thereof:

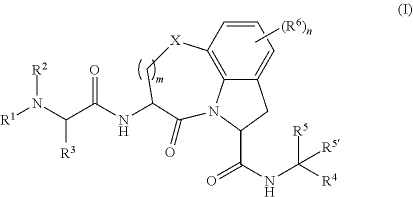

wherein:

R¹ is C₁₋₆ alkyl, C₁₋₆ heteroalkyl, C₃₋₈ cycloalkyl, C₂₋₈ heterocycloalkyl, —C(O)C₁₋₆ alkyl, —C(O)C₁₋₆ heteroalkyl, —C(O)C₃₋₈ cycloalkyl, —C(O)C₂₋₈ heterocycloalkyl, —C(O)aryl, or —C(O)heteroaryl;

R² is C₁₋₆ alkyl, C₁₋₆ alkoxy, or C₁₋₆ heteroalkyl;

R³ is hydrogen, C₁₋₆ alkyl, or C₃₋₈ cycloalkyl;

R⁴ is —C(O)OR, C₁₋₆ alkoxy, C₁₋₆ haloalkyl, —B(OR′)₂, —PO(OR″)₂, or heteroaryl;

each of R⁵ and R^{5′} is independently selected from the group consisting of hydrogen, halo, COOH, hydroxy, C₁₋₆ alkyl, C₁₋₆ alkoxy, and C₁₋₆ haloalkyl;

R⁶ is hydrogen, halo, OH, NRR′, —C(O)OR, —C(O)NRR′, C₁₋₆ alkyl, C₁₋₆ alkoxy, aryl, or heteroaryl;

X is CH₂, O, S, SO₂, or NR;

m is 0, 1, or 2; and

n is 1, 2, or 3,

wherein each of the C₁₋₆ alkyl, C₁₋₆ alkoxy, C₁₋₆ haloalkyl, C₁₋₆ heteroalkyl, C₃₋₈ cycloalkyl, C₂₋₈ heterocycloalkyl, aryl, and heteroaryl is optionally substituted with one or more moieties selected from the group consisting of oxo, halo, OH, CN, CF₃, NH₂, NO₂, C₁₋₆ alkyl, C₁₋₆ haloalkyl, C₁₋₆ thioalkyl, C₃₋₈ cycloalkyl, C2-s heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C7-12 aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′;

wherein each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;

wherein at least one of R¹, R², R³, and R⁶ optionally comprises an imaging agent or a radioisotope.

In other aspects, at least one of R¹, R², R³, and R⁶ of the compound of Formula I, or a pharmaceutically acceptable salt thereof, comprises an imaging agent, such as a paramagnetic ion, an x-ray imaging agent, a fluorophore, and a radioisotope.

In some aspects, the paramagnetic ion is selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

In some aspects, the x-ray imaging agent is selected from the group consisting of lanthanum (III), gold (III), lead (II), bismuth (III), and an iodinated x-ray imaging agent.

In some aspects, the fluorophore is selected from the group consisting of Alexa 350, Alexa 430, AMCA, BODIPY 630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, AF 350, 405, AF532, AF488, AF647, AF680, AF750, Cy5, Cy5.5, Cy7, indocyanine green (ICG), green fluorescent protein (GFP), red fluorescent protein (RFP), and dsRED.

In some embodiments, at least one of R¹, R², R³, and R⁶ of the compound of Formula I, or a pharmaceutically acceptable salt thereof, comprises 1, 2, or 3 independently selected fluorophores.

In some aspects, the radioisotope is selected from the group consisting of ³H, ¹¹C, ¹⁴C, ¹⁸F, ³²P, ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸⁹Zr, ⁹⁰Y, ^{99m}Tc, ¹¹¹In, ¹²³I, ¹²⁴I, ¹²⁵I, ¹³¹I, ¹⁵²Eu, ¹⁵³Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi, ²¹³Bi, and ²²⁵Ac.

In preferred aspects, at least one of R¹, R², R³, and R⁶ of the compound of Formula I or a pharmaceutically acceptable salt thereof, comprises ¹⁸F.

In some aspects, at least one of R¹, R², R³, and R⁶ of the compound of Formula I or a pharmaceutically acceptable salt thereof, comprises an imaging agent selected from the group consisting of a PET imaging agent, a SPECT imaging agent, and a computed tomography imaging agent.

In some embodiments, at least one of R¹, R², R³, and R⁶ of the compound of Formula I or a pharmaceutically acceptable salt thereof is a PET or SPECT imaging agent.

In some embodiments, at least one of R¹, R², R³, and R⁶ of the compound of Formula I or a pharmaceutically acceptable salt thereof, is a PET or SPECT imaging agent comprising a radioisotope selected from the group consisting of ³H, ¹¹C, ¹⁴C, ¹⁸F, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, ²⁰¹Tl.

In a preferred aspect, at least one of R¹, R², R³, and R⁶ of the compound of Formula I or a pharmaceutically acceptable salt thereof, contains a PET imaging agent comprising ¹⁸F.

In some aspects, at least one of R¹, R², R³, and R⁶ of the compound of Formula I or a pharmaceutically acceptable salt thereof, further comprises a chelating agent.

In some embodiments, the chelating agent is selected from the group consisting of 1,4,7-triazacyclononanetriacetic acid (NOTA), 1,4,7-triazacyclononane-1,4-diacetic acid (NODA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and Desferrioxamine B (DFO). In some embodiments, the chelating agent is selected from the group consisting of 1,4,7-triazacyclononanetriacetic acid (NOTA), 1,4,7-triazacyclononane-1,4-diacetic acid (NODA), 1,4,7,10-tetraazacyclododecane-1,4,7,10-tetraacetic acid (DOTA), and 1,4,7-triazacyclononane-1-glutaric acid-4,7-diacetic acid (NODAGA). In some embodiments, the chelating agent is 1,4,7-triazacyclononanetriacetic acid (NOTA).

In some aspects, at least one of R¹, R², R³, and R⁶ of the compound of Formula I or a pharmaceutically acceptable salt thereof, contains an imaging agent comprising one or more of a paramagnetic ion, an x-ray imaging agent, a fluorophore, and a radioisotope and that, the compound of Formula I or a pharmaceutically acceptable salt thereof, binds Granzyme B.

In some aspects, at least one of R¹, R², R³, and R⁶ of the compound of Formula I or a pharmaceutically acceptable salt thereof, comprises a radioisotope and that, the compound of Formula I or a pharmaceutically acceptable salt thereof, binds Granzyme B.

In preferred aspects, at least one of R¹, R², R³, and R⁶ of the compound of Formula I or a pharmaceutically acceptable salt thereof, comprises ¹⁸F and that, the compound of Formula I or a pharmaceutically acceptable salt thereof, binds Granzyme B.

In one aspect, the compound of Formula I or a pharmaceutically acceptable salt thereof, binds Granzyme B.

In other aspect, the compound of Formula I or a pharmaceutically acceptable salt thereof, is an irreversible binder of Granzyme B.

In some aspect, the compound of Formula I or a pharmaceutically acceptable salt thereof, is an inhibitor of Granzyme B.

The present application further provides a method of imaging Granzyme B in a subject comprising:

i) administering to the subject an effective amount of compound of Formula I or a pharmaceutically acceptable salt thereof, wherein at least one of R1, R2, R3, and R6 of the compound of Formula I or a pharmaceutically acceptable salt thereof, comprises an imaging agent and

ii) imaging the subject with a suitable imaging technique, thereby imaging Granzyme B in the subject.

The present application further provides a method of imaging immune response in a cell or tissue sample, comprising:

i) contacting the cell or tissue sample with an effective amount of compound of Formula I or a pharmaceutically acceptable salt thereof, wherein at least one of R¹, R², R³, and R⁶ of the compound of Formula I or a pharmaceutically acceptable salt thereof, comprises an imaging agent and

ii) imaging the cell or tissue sample with a suitable imaging technique, thereby imaging the immune response in the cell or tissue.

The present application further provides a method of imaging immune response in a subject, comprising:

i) administering to the subject an effective amount of compound of Formula I or a pharmaceutically acceptable salt thereof, wherein at least one of R¹, R², R³, and R⁶ of the compound of Formula I or a pharmaceutically acceptable salt thereof, comprises an imaging agent and

ii) imaging the subject with a suitable imaging technique, thereby imaging the immune response in the subject.

The present application further provides a method of monitoring treatment of a disease in a subject, comprising:

i) administering to the subject an effective amount of compound of Formula I or a pharmaceutically acceptable salt thereof, wherein at least one of R¹, R², R³, and R⁶ of the compound of Formula I or a pharmaceutically acceptable salt thereof, comprises an imaging agent and

ii) imaging the subject with a suitable imaging technique.

The present application further provides a method of monitoring an immune response in the treatment of a disease in a subject, comprising:

i) administering to the subject an effective amount of compound of Formula I or a pharmaceutically acceptable salt thereof, wherein at least one of R¹, R², R³, and R⁶ of the compound of Formula I or a pharmaceutically acceptable salt thereof, comprises an imaging agent and

ii) imaging the subject with a suitable imaging technique.

In some embodiments, the method further comprises administering a therapeutic agent, typically prior to step i). In some embodiments, administration of the therapeutic agent induces an immune response cell or tissue sample or subject.

In some embodiments, the therapeutic agent is selected from the group consisting of an anti-inflammatory agent, a steroid, an immunotherapy agent, a chemotherapeutic agent, and a therapeutic antibody. In some embodiments, the therapeutic agent is a chemotherapeutic agent.

Also covered by this disclosure is a pharmaceutical composition comprising a compound of Formula I and a pharmaceutically acceptable carrier.

Still within the scope of this disclosure is a method of treating an immunoregulatory abnormality in a subject in need thereof, the method comprising administering to said patient a compound of Formula I in an amount effective for treating said immunoregulatory abnormality.

### DETAILED DESCRIPTION

Cancer immunotherapies have represented a significant advance in cancer therapy over recent years. Antibodies directed against immune checkpoints such as programmed cell death protein 1 (PD-1) and cytotoxic t lymphocyte-associated protein 4 (CTLA-4) have been approved with positive outcomes for some patients. Research into the field of immune-oncology continues, with strategies including CAR-T cells, vaccines, small molecules, and antibodies under development. Despite the promise of these therapies, they are not a panacea. These immunotherapies can be associated with significant adverse events, which are costly, and the response rates are typically 20-50%, meaning the majority of patients do not respond to therapy. Furthermore, determining an individual patient's response to therapy can be challenging using conventional methods, as response is frequently associated with an immune-cell infiltrate that can make responding tumors appear to grow on anatomic imaging (e.g., CT, MRI), and demonstrate increased avidity with FDG-PET imaging due to the influx of metabolically active immune cells. Given the constraints of current imaging technologies, clinical studies for cancer immunotherapies typically employ overall survival as their study endpoint as opposed to progression-free survival.

Granzyme B, a downstream marker of cytotoxic T-cell activity, could serve as a novel biomarker to assess cancer immunotherapy efficacy. Granzyme B expression within a tumor can be assessed not only for CTL presence or absence, but as an effector protein released by active T-cells that also integrates a measure of CTL activity, thus accounting for issues of T-cell exhaustion that make assessment of CTL presence difficult to accomplish. Accordingly, the present application provides novel Granzyme B specific imaging agents.

### Compounds

This disclosure relates to compounds that bind Granzyme B and can be used for diagnostic and therapeutic purposes, in particular as imaging agents. The compounds are of Formula I or a pharmaceutically acceptable salt thereof.

The compound of Formula I or a pharmaceutically acceptable salt thereof, is as follows:

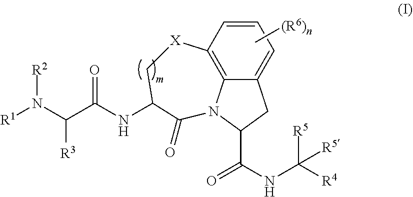

wherein:

R¹ is C₁₋₆ alkyl, C₁₋₆ heteroalkyl, C₃₋₈ cycloalkyl, C₂₋₈ heterocycloalkyl, —C(O)C₁₋₆ alkyl, —C(O)C₁₋₆ heteroalkyl, —C(O)C₃₋₈ cycloalkyl, —C(O)C₂₋₈ heterocycloalkyl, —C(O)aryl, or —C(O)heteroaryl;

R² is C₁₋₆ alkyl, C₁₋₆ alkoxy, or C₁₋₆ heteroalkyl;

R³ is hydrogen, C₁₋₆ alkyl, or C₃₋₈ cycloalkyl;

R⁴ is —C(O)OR, C₁₋₆ alkoxy, C₁₋₆ haloalkyl, —B(OR′)₂, —PO(OR″)₂, or heteroaryl;

each of R⁵ and R^{5′} is independently selected from the group consisting of hydrogen, halo, COOH, hydroxy, C₁₋₆ alkyl, C₁₋₆ alkoxy, and C₁₋₆ haloalkyl;

R⁶ is hydrogen, halo, OH, NRR′, —C(O)OR, —C(O)NRR′, C₁₋₆ alkyl, C₁₋₆ alkoxy, aryl, or heteroaryl;

X is CH₂, O, S, SO₂, or NR;

m is 0, 1, or 2; and

n is 1, 2, or 3,

wherein each of the C₁₋₆ alkyl, C₁₋₆ alkoxy, C₁₋₆ haloalkyl, C₁₋₆ heteroalkyl, C₃₋₈ cycloalkyl, C₂₋₈ heterocycloalkyl, aryl, and heteroaryl is optionally substituted with one or more moieties selected from the group consisting of oxo, halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C7-12 aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′;

wherein each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;

wherein at least one of R¹, R², R³, and R⁶ optionally comprises an imaging agent or a radioisotope.

In some embodiments, compounds of Formula I each feature that X is CH₂.

In some embodiments, compounds of Formula I each feature that X is CH₂ and R¹ is C₁₋₆ alkyl, C₁₋₆ heteroalkyl, —C(O)C₁₋₆ alkyl, or —C(O)C₁₋₆ heteroalkyl.

In some embodiments, compounds of Formula I each feature that X is CH₂ and R² is C₁₋₆ alkyl or C₁₋₆ heteroalkyl.

In some embodiments, compounds of Formula I each feature that X is CH₂ and R³ is C₁₋₆ alkyl.

In some embodiments, compounds of Formula I each feature that X is CH₂ and R⁴ is heteroaryl and each of R⁵ and R^{5′} is hydrogen, wherein the heteroaryl is optionally substituted with 1, 2, or 3 substituents independently selected from the group consisting of oxo, halo, hydroxy, C₁₋₆ alkyl, and C₁₋₆ haloalkyl.

In some embodiments, compounds of Formula I each feature that X is CH₂; R¹ is C₁₋₆ alkyl, C₁₋₆ heteroalkyl, —C(O)C₁₋₆ alkyl, or —C(O)C₁₋₆ heteroalkyl; and R² is C₁₋₆ alkyl or C₁₋₆ heteroalkyl.

In some embodiments, compounds of Formula I each feature that X is CH₂; R³ is C₁₋₆ alkyl, R⁴ is heteroaryl, and each of R⁵ and R^{5′} is hydrogen.

In some embodiments, compounds of Formula I each feature that X is CH₂; R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen. These compounds can have R³ being butyl, e.g.,

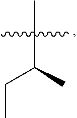 [SMILES: *[C@@H](C)CC]

and R⁴ being unsubstituted triazole, e.g.,

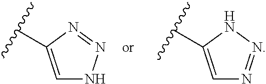 [SMILES: *c1c[nH]nn1 ; *c1cnn[nH]1]

Preferably, these compounds have R¹ being —C(O)C₁₋₆ alkyl or —C(O)C₁₋₆ heteroalkyl.

In some embodiments, compounds of Formula I each feature that X is CH₂ and R¹ is —C(O)C₁₋₆ alkyl substituted with aryl or heteroaryl.

In some embodiments, compounds of Formula I each feature that X is CH₂ and R¹ is —C(O)C₁₋₆ heteroalkyl substituted with F.

In some embodiments, compounds of Formula I each feature that X is O.

In some embodiments, compounds of Formula I each feature that X is O; R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen. In these compounds, R³ can be butyl, e.g.,

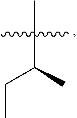 [SMILES: *[C@@H](C)CC]

and R⁴ can be unsubstituted triazole, e.g.,

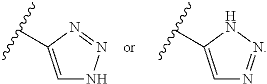 [SMILES: *c1c[nH]nn1 ; *c1cnn[nH]1]

Preferably, these compounds have R¹ being —C(O)C₁₋₆ alkyl or —C(O)C₁₋₆ heteroalkyl.

In some embodiments, compounds of Formula I each feature that X is O and R¹ is —C(O)C₁₋₆ alkyl substituted with aryl or heteroaryl.

In some embodiments, compounds of Formula I each feature that X is O and R¹ is —C(O)C₁₋₆ heteroalkyl substituted with F.

In some embodiments, compounds of Formula I each feature that X is NR, in which R is H, C₁₋₆ alkyl, C₁₋₆ haloalkyl, C₁₋₆ alkoxy, C₃₋₈ cycloalkyl, C₂₋₈ heterocycloalkyl, aryl, or heteroaryl. Preferably, R is H, C₁₋₆ alkyl, or C₃₋₈ cycloalkyl.

In some embodiments, compounds of Formula I each feature that X is NR; R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen. In these compounds, R³ can be butyl, e.g.,

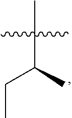 [SMILES: *[C@@H](C)CC]

and R⁴ can be unsubstituted triazole, e.g.,

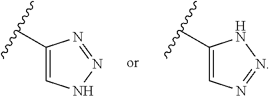 [SMILES: *c1c[nH]nn1 ; "or" ; *c1cnn[nH]1]

Preferably, these compounds have R¹ being —C(O)C₁₋₆ alkyl or —C(O)C₁₋₆ heteroalkyl.

In some embodiments, compounds of Formula I each feature that X is NR and R¹ is —C(O)C₁₋₆ alkyl substituted with aryl or heteroaryl.

In some embodiments, compounds of Formula I each feature that X is NR and R¹ is —C(O)C₁₋₆ heteroalkyl substituted with F.

In some embodiments, the compound of Formula I is a compound of the following formula:

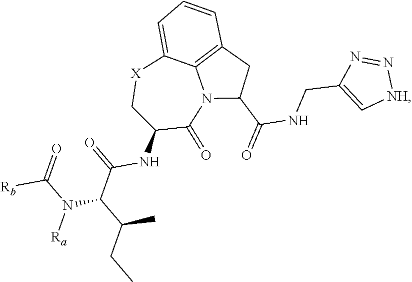

wherein X is CH₂, O, S, SO₂, or NR, in which R is H, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl,

Rₐ is C₁₋₆ alkyl or C₁₋₆ heteroalkyl in which the C₁₋₆ alkyl or C₁₋₆ heteroalkyl is optionally substituted with a halo, heteroaryl, or C(O)ORc,

R_{b} is C₁₋₆ alkyl or C₁₋₆ heteroalkyl in which the C₁₋₆ alkyl or C₁₋₆ heteroalkyl is optionally substituted with a halo, heteroaryl, —C(O)OR_{c}, or —C(O)NR_{c}R_{d},

wherein R_{c} and R_{d}, independently, is H or C₁₋₆ alkyl.

In some embodiments, the moiety

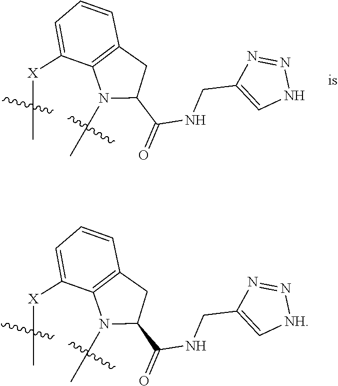

In some embodiments, Rₐ is C₁₋₆ alkyl. In some embodiments, Rₐ is C₁₋₆ alkyl substituted with —C(O)OR_{c}. In some embodiments, Rₐ is C₁₋₆ alkyl substituted with a heteroaryl. In some embodiments, Rₐ is C₁₋₆ alkyl substituted with a benzothiophenyl. In some embodiments, Rₐ is C₁₋₆ heteroalkyl substituted with F.

In some embodiments, R_{b} is C₁₋₆ alkyl. In some embodiments, R_{b} is C₁₋₆ alkyl substituted with —C(O)OR_{c} or —C(O)NR_{c}R_{d}. In some embodiments, R_{b} is C₁₋₆ alkyl substituted with a heteroaryl. In some embodiments, R_{b} is C₁₋₆ alkyl substituted with a heteroaryl selected from imidazolyl, benzothiophenyl, and benzooxazolyl. In some embodiments, R_{b} is C₁₋₆ heteroalkyl substituted with F.

In some embodiments, C₁₋₆ heteroalkyl contains at least one O as the heteroatom connecting two carbons in the alkyl chain.

Exemplary compounds of Formula I include, but are not limited to, the following compounds:

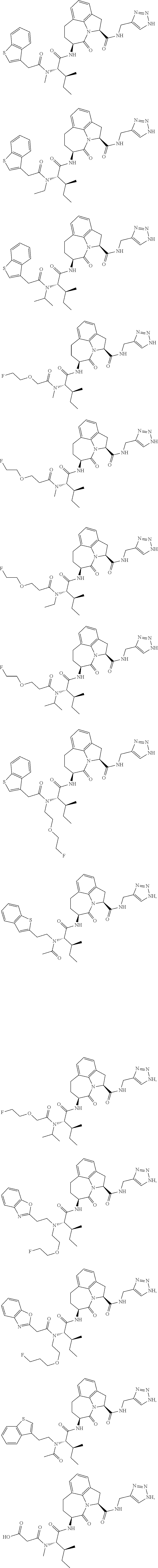

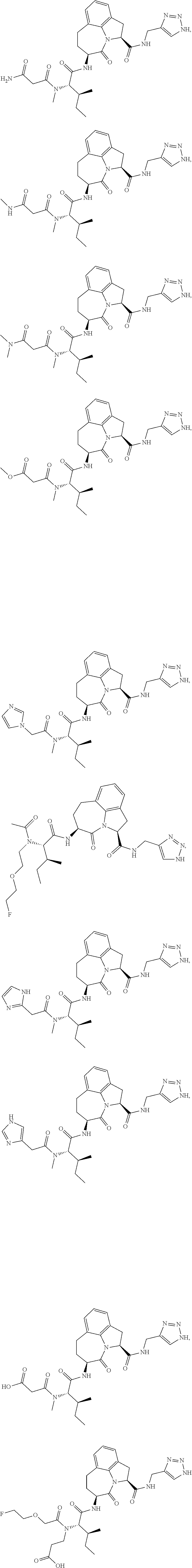

An exemplary compound of Formula I can be one of the following compounds:

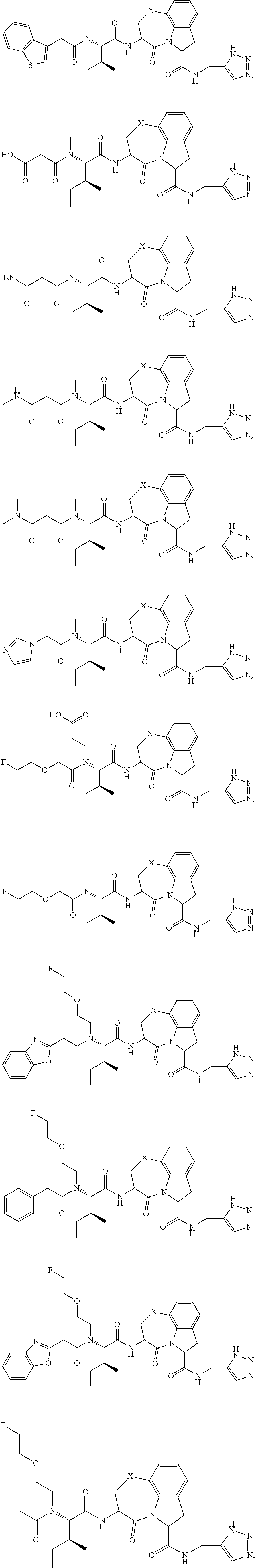

wherein X is CH₂, O, S, SO₂, or NR, in which R is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl.

In some embodiments, each heteroaryl is independently selected from monocyclic 5-6 membered heteroaryl and 8-10 membered bicyclic heteroaryl.

In some embodiments, heterocycloalkyl is independently a monocyclic 4-6 membered heterocycloalkyl, or a bicyclic 8-10 membered heterocycloalkyl group, each comprising 1, 2, 3, or 4 heteroatoms selected from O, S and N, and optionally substituted with 1, 2, or 3 substituents independently selected from the group consisting of C₁₋₄ alkyl, C₁₋₄ haloalkyl, —O—C₁₋₄ alkyl, —O—C₁₋₄ haloalkyl, halo, hydroxy and oxo groups.

In some embodiments, each of heteroaryl and heterocycloalkyl is independently selected from the group consisting of benzimidazolyl, benzofuranyl, benzopyrazolyl, benzotriazolyl, benzothiophenyl, benzoxazolyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, tetrazolyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, tetrahydrofuranyl, and tetrahydrothienyl, each optionally substituted with 1, 2, or 3 substituents independently selected from the group consisting of C₁₋₄ alkyl, C₁₋₄ haloalkyl, —O—C₁₋₄ alkyl, —O—C₁₋₄ haloalkyl, halo, hydroxy and oxo groups.

It is preferred that, in the compound of Formula I or a pharmaceutically acceptable salt thereof, at least one of R¹, R², R³, and R⁶ comprises an imaging agent. If desired, two, three or all of R¹, R², R³, and R⁶ can comprises an imaging agent.

Suitable imaging agents are selected from the group consisting of a paramagnetic ion, an x-ray imaging agent, a fluorophore, and a radioisotope.

Suitable paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), and erbium (III). The paramagnetic ions are either directly or indirectly (e.g., through a chelator) bound to the compounds of Formula I or a pharmaceutically acceptable salt thereof, provided herein.

Suitable x-ray imaging agents include lanthanum (III), gold (III), lead (II), bismuth (III), and iodinated x-ray imaging agents (e.g, diatrizoate, ioxaglate, metrizoate, iopamidol, iohexol, ioxilan, iopromide, iodixanol, and ioversol).

Suitable fluorophores include Alexa 350, Alexa 430, AMCA, BODIPY 630/650, BODIPY 650/665, BODIPY-FL, BODPY-R6G, 13BODLPY-TMR, BODLPY-TRX, cascade blue, Cy3, Cy5, 6-FAM, fluorescein isothiocyanate, HEX, 6-JOE, oregon green 488, oregon green 500, oregon green 514, quantum dots, pacific blue, REG, rhodamine green, rhodamine red, renographin, ROX, TAMRA, TET, tetramethylrhodamine, Texas Red, AF 350, 405, AF532, AF488, AF647, AF680, AF750, Cy5, Cy5.5, Cy7, indocyanine green (ICG), green fluorescent protein (GFP), red fluorescent protein (RFP), and dsRED.

The radioisotopes provided herein are useful as imaging agents in one or more of the methods provided herein. In addition, the radioistopes provided herein may also be useful in one or more therapeutic applications, for example, when administered to a subject in a therapeutically effective amount. For example, ¹³¹I and ⁶⁴Cu may be useful as imaging agents (e.g., as non-toxic and/or non-therapeutic radioisotopes) when administered to the subject at low concentrations (e.g., 5 mCi) and may also be useful as therapeutic agents (i.e., as toxic radioisotopes and/or therapeutic radioisotopes) when administered to the subject at a higher concentration.

Suitable radioisotopes include ³H, ¹¹C, ¹⁴C, ¹⁸F, ³²P, ³⁵S, ³⁶Cl, ⁵¹Cr, ⁵²Fe, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸⁹Zr, ⁹⁰Y, ^{99m}Tc, ¹¹¹In, ¹²³I, ¹²⁴I, ¹²⁵I, ¹³¹I, ¹⁵²Eu, ¹⁵³Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi, ²¹³Bi, and ²²⁵Ac. The radioisotopes are either directly or indirectly (e.g., through a chelator) bound to the compounds of Formula I or a pharmaceutically acceptable salt thereof, provided herein.

In preferred aspects of the disclosure, at least one of R¹, R², R³, and R⁶ of the compound of Formula I or a pharmaceutically acceptable salt thereof, contains ¹⁸F.

In some embodiments, at least one of R¹, R², R³, and R⁶ of the compound of Formula I or a pharmaceutically acceptable salt thereof, comprises an imaging agent selected from the group consisting of a positron emission tomography (PET) imaging agent, a single-photon emission computed tomography (SPECT) imaging agent, and a computed tomography imaging agent.

In some embodiments, the compound of Formula I or a pharmaceutically acceptable salt thereof, is a PET or SPECT imaging agent. In some embodiments, the compound of Formula I or a pharmaceutically acceptable salt thereof, is a PET imaging agent. In some embodiments, the compound of Formula I or a pharmaceutically acceptable salt thereof, is a SPECT imaging agent. In some embodiments, the compound of Formula I or a pharmaceutically acceptable salt thereof, is a computed tomography imaging agent. In some embodiments, the compound of Formula I or a pharmaceutically acceptable salt thereof, is a radioisotopic computed tomography imaging agent.

In some embodiments, the compound of Formula I or a pharmaceutically acceptable salt thereof, is a PET or SPECT imaging agent, wherein at least one of R¹, R², R³, and R⁶ of the compound of Formula I or a pharmaceutically acceptable salt thereof, comprises one or more radioisotopes selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹Ac.

In some embodiments, the compound of Formula I or a pharmaceutically acceptable salt thereof, is a PET or SPECT imaging agent, wherein at least one of R¹, R², R³, and R⁶ of the compound of Formula I or a pharmaceutically acceptable salt thereof, comprises 1, 2, or 3 radioisotopes selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹Tl.

In some embodiments, the compound of Formula I or a pharmaceutically acceptable salt thereof, is a PET or SPECT imaging agent, wherein at least one of R¹, R², R³, and R⁶ of the compound of Formula I or a pharmaceutically acceptable salt thereof, comprises one radioisotope selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹Tl.

In some embodiments, the compound of Formula I or a pharmaceutically acceptable salt thereof, is a PET or SPECT imaging agent, wherein at least one of R¹, R², R³, and R⁶ of the compound of Formula I or a pharmaceutically acceptable salt thereof, comprises ⁶⁸Ga.

In a preferred embodiment, the compound of Formula I or a pharmaceutically acceptable salt thereof, is a PET imaging agent, wherein at least one of R¹, R², R³, and R⁶ of the compound of Formula I or a pharmaceutically acceptable salt thereof, comprises ¹⁸F.

In some embodiments, at least one of R¹, R², R³, and R⁶ of the compound of Formula I or a pharmaceutically acceptable salt thereof, further comprises one or more chelating agents.

Suitable chelating agents include, but are not limited to, 1,4,7-triazacyclononanetriacetic acid (NOTA), 1,4,7-triazacyclononane-1,4-diacetic acid (NODA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and Desferrioxamine B (DFO). In some embodiments, the chelating agent is selected from the group consisting of 1,4,7-triazacyclononanetriacetic acid (NOTA), 1,4,7-triazacyclononane-1,4-diacetic acid (NODA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and Desferrioxamine B (DFO). In some embodiments, the chelating agent is selected from the group consisting of 1,4,7-triazacyclononanetriacetic acid (NOTA), 1,4,7-triazacyclononane-1,4-diacetic acid (NODA), 1,4,7,10-tetraazacyclododecane-1,4,7,10-tetraacetic acid (DOTA), and 1,4,7-triazacyclononane-1-glutaric acid-4,7-diacetic acid (NODAGA). In some embodiments, the chelating agent is 1,4,7-triazacyclononanetriacetic acid (NOTA).

### Synthesis

As will be appreciated, the compounds provided herein, including salts thereof, can be prepared using known organic synthesis techniques and can be synthesized according to any of numerous possible synthetic routes.

The compound of Formula I or a pharmaceutically acceptable salt thereof, provided herein can be prepared by the routes depicted in the Scheme 1 below. Appropriate protective groups for use in such syntheses will be found in the above texts, as well as in McOmie, Protective Groups in Organic Chemistry, (1973):98. Additional reactions may be necessary, as described elsewhere, to form intramolecular linkages to restrain conformation.

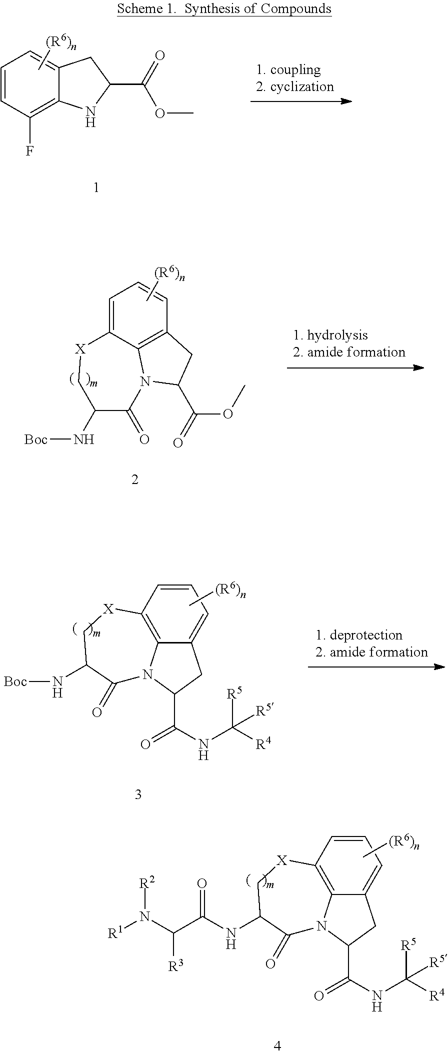

Some compounds of the disclosure could be synthesized following the general Scheme 1 wherein Compound 1 could be coupled with an appropriate protected amino acid and followed with cyclization onto the indole nitrogen to form Compound 2. The ester of Compound 2 could then be hydrolyzed followed by amide formation to form Compound 3. Boc deprotection of Compound 3 could be followed by amide bond formation using an appropriate acid to form final Compound 4.

### Imaging Agents

Many appropriate imaging agents are known in the art (see, for e.g., U.S. Pat. Nos. 5,021,236; 4,938,948; and 4,472,509, the disclosure of each of which is incorporated herein by reference in its entirety). Radioactively labeled compounds of Formula I or a pharmaceutically acceptable salt thereof, provided herein may be prepared according to well-known methods in the art. As an example, compounds of Formula I or a pharmaceutically acceptable salt thereof, provided herein may be labeled with ⁶⁸Ga by radiometalation of a bifunctional chelator provided herein (e.g., NOTA, NODA, DOTA, or NODAGA) or a similar derivative thereof.

Synthetic methods for incorporating radioisotopes into organic compounds are well known in the art, and one of ordinary skill in the art will readily recognize other methods applicable for the compounds provided herein.

It will be appreciated by one skilled in the art that the processes described are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Definitions

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

Throughout the definitions, the term “Cn-m” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. In some embodiments, the C₁₋₄ alkyl group is optionally substituted with 1, 2, or 3 halo groups. Preferably, the C₁₋₄ alkyl group is optionally substituted with 1, 2, or 3 fluoro groups.

As used herein, the term “Cₙ₋ₘ heteroalkyl,” employed alone or in combination with other terms, refers to a saturated hydrocarbon group that may be straight-chain or branched, having n to m carbons, wherein at least one heteroatom selected from nitrogen, sulfur and oxygen connects two carbon atoms in the saturated hydrocarbon chain (straight or branched).

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “halo” or “halogen” refers to F, C1, Br, or I, or their isotopes. In some embodiments, a halo or halogen is F, Cl, or Br, or their isotopes. Preferably, the halo or halogen is F or ¹⁸F.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

As used herein, the term “hydroxy” refers to a group of formula —OH.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “oxo” refers to a double-bonded oxygen (i.e., ═O).

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of Formula I depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds of Formula I or a pharmaceutically acceptable salt thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

### Methods of Use

The present application further provides methods of imaging Granzyme B using a compound of Formula I or a pharmaceutically acceptable salt thereof.

In some embodiments, the method of imaging is performed in a cell, a tissue, a cell sample, a tissue sample, or a subject.

As used herein, the term “subject,” refers to any animal, including mammals and invertebrates. For example, mice, rats, other rodents, rabbits, dogs, cats, swine, cattle, sheep, horses, primates, fish, and humans. In some embodiments, the subject is a human. In some embodiments, the subject is a mouse. In some embodiments, the subject is a fish (e.g., a zebra fish).

In some embodiments, the method comprises administering to the subject an effective amount of a compound of Formula I or a pharmaceutically acceptable salt thereof. In some embodiments, the method is an in vitro method. In some embodiments, the method is an in vivo method.

The present application further provides a method of imaging Granzyme B in a cell or tissue, comprising:

i) contacting the cell or tissue with an effective amount of a compound of Formula I or a pharmaceutically acceptable salt thereof, and

ii) imaging the cell or tissue with a suitable imaging technique, thereby imaging Granzyme B in the cell or tissue, wherein:

at least one of R¹, R², R³, and R⁶ of the compound of Formula I or a pharmaceutically acceptable salt thereof, comprises an imaging agent.

The present application further provides a method of imaging Granzyme B in a cell sample or tissue sample, comprising:

i) contacting the cell sample or tissue sample with effective amount of a compound of Formula I or a pharmaceutically acceptable salt thereof, and

ii) imaging the cell sample or tissue sample with a suitable imaging technique, thereby imaging Granzyme B in the cell sample or tissue sample, wherein:

at least one of R¹, R², R³, and R⁶ of the compound of Formula I or a pharmaceutically acceptable salt thereof, comprises an imaging agent.

The present application further provides a method of imaging Granzyme B in a subject, comprising:

i) administering to the subject an effective amount of a compound of Formula I or a pharmaceutically acceptable salt thereof, and

ii) imaging the subject with a suitable imaging technique, thereby imaging Granzyme B in the subject, wherein:

at least one of R¹, R², R³, and R⁶ of the compound of Formula I or a pharmaceutically acceptable salt thereof, comprises an imaging agent.

The present application further provides a method of imaging an immune response in a cell or tissue sample, comprising:

i) contacting the cell or tissue sample with an effective amount of a compound of Formula I or a pharmaceutically acceptable salt thereof, and

ii) imaging the cell or tissue sample with a suitable imaging technique, thereby imaging the immune response in the cell or tissue sample, wherein:

at least one of R¹, R², R³, and R⁶ of the compound of Formula I or a pharmaceutically acceptable salt thereof, comprises an imaging agent.

The present application further provides a method of imaging an immune response in a subject, comprising:

i) administering to the subject an effective amount of a compound of Formula I or a pharmaceutically acceptable salt thereof, and

ii) imaging the subject with a suitable imaging technique, thereby imaging the immune response in the subject, wherein:

at least one of R¹, R², R³, and R⁶ of the compound of Formula I or a pharmaceutically acceptable salt thereof, comprises an imaging agent.

The present application further provides a method of monitoring treatment of a disease in a subject, comprising:

i) administering to the subject an effective amount of a compound of Formula I or a pharmaceutically acceptable salt thereof, and

ii) imaging the subject with a suitable imaging technique, wherein:

at least one of R¹, R², R³, and R⁶ of the compound of Formula I or a pharmaceutically acceptable salt thereof, comprises an imaging agent.

The present application further provides a method of monitoring an immune response in the treatment of a disease in a subject, comprising:

i) administering to the subject an effective amount of a compound of Formula I or a pharmaceutically acceptable salt thereof, and

ii) imaging the subject with a suitable imaging technique, wherein:

at least one of R¹, R², R³, and R⁶ of the compound of Formula I or a pharmaceutically acceptable salt thereof, comprises an imaging agent.

In some embodiments, the methods provided herein further comprise waiting a time sufficient to allow the compound of Formula I or a pharmaceutically acceptable salt thereof, to accumulate at a cell or tissue site (e.g., a cell or tissue site in a subject) associated with the disease, prior to imaging.

In some embodiments, the methods provided herein further comprise waiting a time sufficient to allow the compound of Formula I or a pharmaceutically acceptable salt thereof, to bind Granzyme B at a cell or tissue site (e.g., a cell or tissue site in a subject) associated with the disease, prior to imaging.

In some embodiments, the time sufficient is from about 30 seconds to about 24 hours, for example, about 30 seconds to about 24 hours, about 30 seconds to about 12 hours, about 30 seconds to about 6 hours, about 30 seconds to about 2 hours, about 30 seconds to about 1 hour, about 30 seconds to about 30 minutes, about 30 seconds to about 10 minutes, about 10 minutes to about 24 hours, about 10 minutes to about 12 hours, about 10 minutes to about 6 hours, about 10 minutes to about 2 hours, about 10 minutes to about 1 hour, about 10 minutes to about 30 minutes, about 30 minutes to about 24 hours, about 30 minutes to about 12 hours, about 30 minutes to about 6 hours, about 30 minutes to about 2 hours, about 30 minutes to about 1 hour, about 1 hour to about 24 hours, about 1 hour to about 12 hours, about 1 hour to about 6 hours, about 1 hour to about 2 hours, about 2 hours to about 24 hours, about 2 hours to about 12 hours, about 2 hours to about 6 hours, about 6 hours to about 24 hours, about 6 hours to about 12 hours, or about 12 hours to about 24 hours.

In some embodiments, the suitable imaging technique is a non-invasive imaging technique. In some embodiments, the suitable imaging technique is a minimally invasive imaging technique. As used herein, the term “minimally invasive imaging technique” comprises imaging techniques employing the use of an internal probe or injection of a compound of Formula I or a pharmaceutically acceptable salt thereof, or radiotracer via syringe.

Example imaging techniques include, but are not limited to, fluoroscopic imaging, x-ray imaging, magnetic resonance imaging (MRI), ultrasound imaging, photoacoustic imaging, thermographic imaging, tomographic imaging, echocardiographic imaging, positron emission tomography (PET) imaging, PET with computed tomography (CT) imaging, PET-MRI, single-photon emission computed tomography (SPECT), and ultrasound imaging. In some embodiments, the suitable imaging technique is selected from the group consisting of PET imaging, PET-CT, PET-MRI, and SPECT.

In some embodiments, the suitable imaging technique is selected from the group consisting of positron emission tomography (PET) imaging, positron emission tomography (PET) with computed tomography imaging, and positron emission tomography (PET) with magnetic resonance imaging (MRI). In some embodiments, the suitable imaging technique is selected positron emission tomography (PET) imaging.

Also covered by this disclosure is a method of treating an immunoregulatory abnormality in a subject in need thereof, the method comprising administering to said patient a compound of Formula I in an amount effective for treating said immunoregulatory abnormality.

In some embodiments, an immunoregulatory abnormality as described herein is selected from the group consisting of an autoimmune disorder, an inflammatory disorder, a skin disorder, cancer, and a cardiovascular disorder.

In some embodiments, the immunoregulatory abnormality is a cancer. In some embodiments, the cancer comprises a solid tumor. In some embodiments, the cancer is a hematological cancer (e.g., leukemia, lymphoma, and the like). In some embodiments, the cancer is selected from the group consisting of brain, breast cancer, cervical cancer, colorectal cancer, lung cancer, lymphoma, melanoma, bladder cancer, renal cell carcinoma, multiple myeloma, pancreatic cancer, and prostate cancer. In some embodiments, the cancer is selected from the group consisting of Hairy-cell leukemia, Kaposi's sarcoma, follicular lymphoma, chronic myeloid leukemia, cutaneous T-cell lymphoma, peripheral T-cell lymphoma, T-cell prolymphocytic leukemia, Classical Hodgkin's lymphoma, B-cell non-Hodgkin's lymphoma, chronic lymphocytic leukemia, acute myeloid leukemia, myelodysplastic syndrome, primary myelofibrosis, post-essential thrombocytheia myelofibrosis, post-polycythemia vera myelofibrosis, melanoma, renal cell carcinoma, prostate cancer, non-small cell lung cancer, small cell lung cancer, glioblastoma, hepatocellular carcinoma, urothelial carcinoma, esophageal carcinoma, gastroesophageal carcinoma, gastric cancer, multiple myeloma, colon cancer, rectal cancer, squamous cell carcinoma of the head and neck, epithelial ovarian cancer (EOC), primary peritoneal cancer, fallopian tube carcinoma, HER2+ breast cancer, ER+/PR+/HER2− breast cancer, triple-negative breast cancer, gastric cancer, pancreatic cancer, bladder cancer, Merkel cell cancer, nasopharyngeal cancer, adrenocortical carcinoma, meningioma, neuroblastoma, retinoblastoma, osteosarcoma, rhabdomyosarcoma, Ewing's sarcoma, liposarcoma, fibrosarcoma, leiomyosarcoma, peripheral primitive neuroectodermal tumor, squamous cell carcinoma of the cervix, squamous cell carcinoma of the vagina, and squamous cell carcinoma of the vulva. In some embodiments, the cancer is colon cancer. In some embodiments, the cancer is melanoma.

In some embodiments, the immunoregulatory abnormality is selected from the group consisting of graft-versus-host disease, rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, rheumatic fever, post-infectious glomerulonephritis, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia, alopecia senilis by preventing epilation, alopecia senilis by providing hair germination and/or promoting hair generation and hair growth, muscular dystrophy, pyoderma, Sezary's syndrome, Addison's disease, ischemia-reperfusion injury of organs, transplantation disease, ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, aging, carcinogenesis, metastasis of carcinoma and hypobaropathy, histamine or leukotriene-C4 release associated diseases, Behcet's disease, autoimmune hepatitis, primary biliary cirrhosis, sclerosing cholangitis, partial liver resection, acute liver necrosis, necrosis caused by toxin, viral hepatitis, shock, anoxia, B-virus hepatitis, non-A/non-B hepatitis, cirrhosis, alcoholic cirrhosis, hepatic failure, fulminant hepatic failure, late-onset hepatic failure, acute-on-chronic liver failure, cytomegalovirus infection, HCMV infection, AIDS, senile dementia, trauma, chronic bacterial infection, malignancy of lymphoid origin, acute lymphocytic leukemia, chronic lymphocytic leukemia, acute lymphocytic lymphoma, and chronic lymphocytic lymphoma.

In some embodiments, the immunoregulatory abnormality is selected from the group consisting of systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

In some embodiments, the immunoregulatory abnormality is selected from the group consisting of bone marrow rejection, organ transplant rejection, and graft-versus-host disease.

As used herein, the phrase “therapeutically effective amount” refers to the amount of active compound of Formula I or a pharmaceutically acceptable salt thereof, or pharmaceutical agent that elicits the biological or medicinal response that is being sought in a tissue, system, animal, individual or human by a researcher, veterinarian, medical doctor or other clinician.

In some embodiments, the dosage of the compound of Formula I or a pharmaceutically acceptable salt thereof, administered to a subject or individual is about 1 μg to about 2 g, for example, about 1 μg to about 2 g, about 1 μg to about 1000 mg, about 1 μg to about 500 mg, about 1 μg to about 100 mg, about 1 μg to about 50 mg, about 1 μg to about 1 mg, about 1 μg to about 500 μg, about 1 μg to about 100 μg, about 1 μg to about 10 μg, about 10 μg to about 2 g, for example, about 10 μg to about 2 g, about 10 μg to about 1000 mg, about 10 μg to about 500 mg, about 10 μg to about 100 mg, about 10 μg to about 50 mg, about 10 μg to about 1 mg, about 10 μg to about 500 μg, about 10 μg to about 100 μg, about 100 μg to about 2 g, for example, about 100 μg to about 2 g, about 100 jig to about 1000 mg, about 100 μg to about 500 mg, about 100 μg to about 100 mg, about 100 μg to about 50 mg, about 100 μg to about 1 mg, about 100 μg to about 500 μg, about 500 μg to about 2 g, for example, about 500 μg to about 2 g, about 500 μg to about 1000 mg, about 500 μg to about 500 mg, about 500 μg to about 100 mg, about 500 μg to about 50 mg, about 500 μg to about 1 mg, about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

### Combination Therapies

When employed in methods of treating a disease, the compound of Formula I or a pharmaceutically acceptable salt thereof, provided herein can be administered in combination with one or more of the additional agents provided herein. Example therapeutic agents include, but are not limited to, anti-inflammatory agents, steroids, immunotherapy agents, chemotherapeutic agents, and therapeutic antibodies.

In some embodiments, administration of the therapeutic agent induces an immune response cell or tissue sample or subject. In some embodiments, the therapeutic agent is a compound of Formula I or a pharmaceutically acceptable salt thereof.

In some embodiments, the therapeutic agent is a compound of Formula I or a pharmaceutically acceptable salt thereof, wherein at least one of R¹, R², R³, and R⁶ of the compound of Formula I or a pharmaceutically acceptable salt thereof, comprises a radioisotope (e.g., a therapeutic radioisotope).

In some embodiments, the therapeutic agent is a compound of Formula I or a pharmaceutically acceptable salt, wherein at least one of R¹, R², R³, and R⁶ of the compound of Formula I or a pharmaceutically acceptable salt thereof, comprises a toxic radioisotope.

Examples of toxic radioisotopes include, but are not limited to, alpha emitters (e.g., ²¹¹At, ²¹²Pb, ²¹²Bi, ²¹³Bi, ²²⁵Ac, ²²⁷Th) and beta emitters (e.g., ⁹⁰Y, ¹³¹I and ¹⁷⁷Lu). In some embodiments, the toxic radioisotope is a beta emitter.

In some embodiments, the toxic radioisotope is a beta emitter selected from the group consisting of ⁹⁰Y, ¹³¹I and ¹⁷⁷Lu. In some embodiments, the toxic radioisotope is an alpha emitter.

In some embodiments, the toxic radioisotope is an alpha emitter selected from the group consisting of ²¹¹At, ²¹²Pb, ²¹²Bi, ²¹³Bi, ²²⁵Ac, ²²⁷Th.

The present application further provides a method of treating a disease in a subject, comprising:

i) administering to the subject, an effective amount of compound of Formula I or a pharmaceutically acceptable salt thereof, wherein at least one of R¹, R², R³, and R⁶ of the compound of Formula I or a pharmaceutically acceptable salt thereof, comprises a non-toxic imaging agent (e.g., a non-toxic radioisotope); and

ii) imaging the subject with a suitable imaging technique, thereby treating the disease in the subject.

The present application further provides a method of treating a disease in a subject, comprising:

i) administering to the subject, an effective amount of compound of Formula I or a pharmaceutically acceptable salt thereof, wherein at least one of R¹, R², R³, and R⁶ of the compound of Formula I or a pharmaceutically acceptable salt thereof, comprises a non-therapeutic imaging agent (e.g., a non-therapeutic radioisotope); and

ii) imaging the subject with a suitable imaging technique, thereby treating the disease in the subject.

In some embodiments, at least one of R¹, R², R³, and R⁶ of the compound of Formula I or a pharmaceutically acceptable salt thereof, comprises a non-toxic radioisotope selected from the group consisting of ³H, ¹¹C, ¹⁴C, ¹⁸F, ³²P, ³⁵S, ⁵²Fe, ⁵⁸Co, ⁶⁴Cu, ⁶⁸Ga, ⁷⁶Br, ⁷⁷Br, ⁸⁹Zr, ¹¹¹In, ¹²³I, ¹²⁴I, ¹²⁵I, ¹³¹I, ¹⁸⁶Re, ¹⁸⁸Re, and ²⁰¹Tl.

In a preferred embodiment, at least one of R¹, R², R³, and R⁶ of the compound of Formula I or a pharmaceutically acceptable salt thereof, comprises BF.

In some embodiments, at least one of R¹, R², R³, and R⁶ of the compound of Formula I or a pharmaceutically acceptable salt thereof, comprises a toxic radioisotope selected from the group consisting of ²¹At, ²¹²Pb, ²¹²Bi, ²¹³Bi, ²²⁵Ac, ²²⁷Th, ⁹⁰Y, ¹³¹I and ¹⁷⁷Ln.

In some embodiments, the subject has been identified and/or diagnosed as having the disease to be treated prior to step i). In some embodiments, the subject is identified and/or diagnosed as having the disease to be treated after step ii). For example, the disease to be treated is selected from the group consisting of: an autoimmune disorder, an inflammatory disorder, a skin disorder, cancer, and a cardiovascular disorder as described herein.

In some embodiments, the subject has been treated with one or more immunotherapeutic agents prior to step i). In some embodiments, the disease has been determined to be resistant to the one or more immunotherapeutic agents administered prior to step i).

In some embodiments, the method further comprises:

iii) administering one or more immunotherapeutic agents after the administration of an effective amount of the compound of Formula I or a pharmaceutically acceptable salt thereof. In some embodiments, steps i)-iii) are repeated multiple times.

In some embodiments, the disease to be treated is selected from the group consisting of an autoimmune disorder, an inflammatory disorder, a skin disorder, cancer, and a cardiovascular disorder as described herein. In some embodiments, the disease is a cancer described herein.

In some embodiments, the compound of Formula I or a pharmaceutically acceptable salt thereof, is administered to the subject in a therapeutically effective amount.

In some embodiments, the methods provided herein further comprise administering a therapeutic agent prior to step i). In some embodiments, the methods provided herein further comprise administering a therapeutic agent after step ii).

In some embodiments, the methods provided herein further comprise the steps of:

iii) administering a therapeutically effective amount of a therapeutic agent after step ii); and

iv) repeating steps i) and ii) of the methods provided herein.

In some embodiments, the therapeutic agent is a compound other than a compound of Formula I, or a pharmaceutically acceptable salt thereof, provided herein.

In some embodiments, steps i)-iv) are repeated multiple times.

In some embodiments, the compound of Formula I or a pharmaceutically acceptable salt thereof, provided herein and the one or more additional therapeutic agents are administered according to a dosing regimen over a period of time. In some embodiments, the cell, cell sample, tissue, tissue sample, or subject are imaged with an appropriate imaging technique after administration of an effective amount of compound of Formula I or a pharmaceutically acceptable salt thereof, provided herein. In some embodiments, the cell, cell sample, tissue, tissue sample, or subject are imaged with an appropriate imaging technique after administration of the additional therapeutic agent.

In some embodiments, the present application provides a method of treating a disease in a subject, comprising:

i) administering to the subject an effective amount of a compound of Formula I or a pharmaceutically acceptable salt thereof, wherein at least one of R¹, R², R³, and R⁶ of the compound of Formula I or a pharmaceutically acceptable salt thereof, comprises a non-toxic imaging agent (e.g., a non-toxic radioisotope);

ii) imaging the subject with a suitable imaging technique;

iii) administering to the subject a therapeutic agent, thereby treating the disease in the subject.

In some embodiments, the present application provides a method of treating a disease in a subject, comprising:

i) administering to the subject an effective amount of a compound of Formula I or a pharmaceutically acceptable salt thereof, wherein at least one of R¹, R², R³, and R⁶ of the compound of Formula I or a pharmaceutically acceptable salt thereof, comprises a non-therapeutic imaging agent (e.g., a non-therapeutic radioisotope);

ii) imaging the subject with a suitable imaging technique;

iii) administering to the subject a therapeutic agent, thereby treating the disease in the subject.

In some embodiments, the method further comprises determining if the compound of Formula I or a pharmaceutically acceptable salt thereof, binds to a cell or tissue of the subject to be treated prior to step iii). In some embodiments, the method further comprises determining if the compound of Formula I or a pharmaceutically acceptable salt thereof, binds to Granzyme B, prior to the administration of step iii).

In some embodiments, the subject has been identified and/or diagnosed as having the disease to be treated prior to step i). In some embodiments, the subject is identified and/or diagnosed as having the disease to be treated after step ii).

In some embodiments, the subject has been treated with one or more immunotherapeutic agents prior to step i). In some embodiments, the disease has been determined to be resistant to the one or more immunotherapeutic agents administered prior to step i).

In some embodiments, the method further comprises:

iv) administering one or more immunotherapeutic agents after the administration of the therapeutic agent of step iii). In some embodiments, steps i)-iv) are repeated multiple times.

In some embodiments, the additional therapeutic agent is administered to the subject in a therapeutically effective amount.

In some embodiments, the therapeutic agent is an antibody. Example antibodies for use in combination therapy include but are not limited to trastuzumab (e.g. anti-HER2), ranibizumab (e.g. anti-VEGF-A), bevacizumab (e.g. anti-VEGF), panitumumab (e.g. anti-EGFR), cetuximab (e.g. anti-EGFR), rituxan (anti-CD20), antibodies directed to c-MET, and antibody inhibitors of Granzyme B (e.g., Clone GB11, Clone GrB-7, and NCL-L-Gran-B), ipilimumab (anti-CTLA-4), nivolumab (anti-PD-1), pembrolizumab (anti-PD-1), atezolizumab (anti-PD-1), elotuzumab (anti-SLAM7), and daratumumab (anti-CD38).

In some embodiments, the therapeutic agent is a steroid. Example steroids include corticosteroids such as cortisone, dexamethasone, hydrocortisone, methylprednisolone, prednisolone, and prednisone. In some embodiments, the additional agent is a corticosteroid.

In some embodiments, the therapeutic agent is an anti-inflammatory compound. Example anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

In some embodiments, the therapeutic agent is chemotherapeutic agent. Example chemotherapeutic agents include, but are not limited to, a cytostatic agent, cisplatin, doxorubicin, taxol, etoposide, irinotecan, topotecan, paclitaxel, docetaxel, epothilones, tamoxifen, 5-fluorouracil, methotrexate, temozolomide, cyclophosphamide, SCH 66336, R115777, L778,123, BMS 214662, gefitinib, erlotinib hydrochloride, antibodies to EGFR, imatinib mesylate, intron, ara-C, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, vinorelbine, porfimer, erbitux, liposomal, thiotepa, altretamine, melphalan, trastuzumab, fulvestrant, exemestane, ifosfamide, rituximab, C225, alemtuzumab, clofarabine, cladribine, aphidicolin, sunitinib, dasatinib, tezacitabine, Sml1, triapine, didox, trimidox, amidox, 3-AP, MDL-101,731, bendamustine, ofatumumab, and GS-1101 (also known as CAL-101).

In some embodiments, the chemotherapeutic agent is selected from the group consisting of an alkylating agent (e.g., busulfan, chlorambucil, cisplatin, cyclophosphamide (cytoxan), dacarbazine, ifosfamide, mechlorethamine (mustargen), and melphalan), a nitrosourea (e.g., carmustine, lomustine, semustine, and streptozocin), a triazine (e.g., dacarbazine) an anti-metabolite (e.g., 5-fluorouracil (5-FU), cytarabine (Ara-C), fludarabine, gemcitabine, and methotrexate), a purine analog (e.g., 6-mercaptopurine, 6-thioguanine, and pentostatin (2-deoxycoformycin)), a mitotic inhibitor (e.g., docetaxel, etoposide (VP16), teniposide, paclitaxel, taxol, vinblastine, vincristine, and vinorelbine), an anti-tumor antibiotic (e.g., bleomycin, dactinomycin, daunorubicin, doxorubicin, mitomycin, plicamycin, and idarubicin), a platinum chemotherapeutic agent (e.g., cisplatin and carboplatin), an anthracenedione (e.g., mitoxantrone), a toxin (e.g., ricin A-chain (Burbage, Leukemia research, 21.7 (1997): 681-690), diphtheria toxin A (Massuda et al., Proceedings of the National Academy of Sciences, 94.26 (1997): 14701-14706; Lidor, American journal of obstetrics and gynecology, 177.3 (1997): 579-585), pertussis toxin A subunit, E. coli enterotoxin toxin A subunit, cholera toxin A subunit and Pseudomonas toxin c-terminal), and a gene therapy vector (e.g., a signal transducing protein (e.g., Src, Abl, and Ras), Jun, Fos, and Myc).

In some embodiments, the therapeutic agent is an immunotherapeutic agent. An immunotherapeutic agent generally triggers immune effector cells and molecules to target and destroy cells (e.g., cancer cells). The immune effector may be, for example, an antibody specific for a marker on the surface of a cell (e.g. a tumor cell). The antibody alone may serve as an effector of therapy or it may recruit other cells to effect cell killing. Various effector cells include, but are not limited to, cytotoxic T cells and NK cells.

Example immunotherapeutic agents include, but are not limited to, azathioprine, chlorambucil, cyclophosphamide, cyclosporine, daclizumab, infliximab, methotrexate, tacrolimus, immune stimulators (e.g., IL-2, IL-4, IL-12, GM-CSF, tumor necrosis factor; interferons alpha, beta, and gamma; F42K and other cytokine analogs; a chemokine such as MIP-1, MIP-1β, MCP-1, RANTES, IL-8; or a growth factor such FLT3 ligand), an antigenic peptide, polypeptide or protein, or an autologous or allogenic tumor cell composition (see e.g., Ravindranath & Morton, International reviews of immunology, 7.4 (1991): 303-329), hormonal therapy, adrenocorticosteroids, progestins (e.g., hydroxyprogesterone caproate, medroxyprogesterone acetate, and megestrol acetate), estrogens (e.g., diethylstilbestrol and ethinyl estradiol), anti-estrogens (e.g., testosterone propionate and fluoxymesterone), anti-androgens (e.g., flutamide), and gonadotropin-releasing hormone analogs (e.g., leuprolide). Additional immunotherapeutic agents are known in the art, and can be found, for example, in Rosenberg et al, New England Journal of Medicine, 319.25 (1988): 1676-1680; and Rosenberg et al, Annals of surgery, 210.4 (1989): 474).

The therapeutic agents provided herein can be effective over a wide dosage range and are generally administered in an effective amount. It will be understood, however, that the amount of the therapeutic agent actually administered will usually be determined by a physician, according to the relevant circumstances, including the condition to be imaged, the chosen route of administration, the actual compound administered, the age, weight, and response of the individual subject, the severity of the subject's symptoms, and the like.

### Pharmaceutical Compositions

Still within the scope of this disclosure is a pharmaceutical composition comprising a compound of Formula I and a pharmaceutically acceptable carrier.

When employed as pharmaceuticals, the compound of Formula I or a pharmaceutically acceptable salt thereof, and therapeutic agents provided herein can be administered in the form of pharmaceutical compositions. These compositions can be prepared as described herein or elsewhere, and can be administered by a variety of routes, depending upon whether local or systemic treatment is desired and upon the area to be treated.

Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral.

Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump.

In some embodiments, the compound of Formula I or a pharmaceutically acceptable salt thereof, provided herein is suitable for parenteral administration. In some embodiments, the compound of Formula I or a pharmaceutically acceptable salt thereof, is suitable for intravenous administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the pharmaceutical compositions provided herein are suitable for parenteral administration. In some embodiments, the pharmaceutical compositions provided herein are suitable for intravenous administration.

Also provided are pharmaceutical compositions which contain, as the active ingredient, a compound provided herein, or a pharmaceutically acceptable salt thereof, in combination with one or more pharmaceutically acceptable carriers (excipients).

In making the pharmaceutical compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

Thus, the pharmaceutical compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

### Kits

The present application further provides a kit comprising a compound of Formula I or a pharmaceutically acceptable salt thereof. In some embodiments, the kit further comprises one or more additional therapeutic agents provided herein.

In some embodiments, the kit comprises one or more components of the compounds provided herein (e.g., one or more imaging agents, one or more chelating agents, one or more linking groups, or compounds of Formula I or a pharmaceutically acceptable salt thereof, that bind Granzyme B).

In some embodiments, each component of the kit (is stored within the kit in a separate container (e.g., a separate vial). In some embodiments, the components of the kits may be packaged either in aqueous media or in lyophilized form.

In some embodiments, the kit further comprises instructions, for example, as inserts or as labels, indicating quantities of the composition to be administered, guidelines for administration, and/or guidelines for mixing components of the kit to prepare a compound of Formula I, or a pharmaceutically acceptable salt thereof. In some embodiments, the instructions further comprise instructions for performing one or more of the methods provided herein.

The kits provided herein can further include, if desired, one or more conventional pharmaceutical kit components, such as, for example, containers with one or more pharmaceutically acceptable carriers, additional containers, etc., as will be readily apparent to those skilled in the art.

The compounds of Formula I described above can be tested using the human Granzyme B biochemical assay provided below.

### Assay Parameters:

TABLE 1
Specific assay parameters

| | |
|---|---|
| Enzyme : Granzyme B (human lymphocytes) | 5 nM |
| Substrate : Ac-IETD-AFC | 200 μM |
| Cpd incubation with enzyme | 30 mins |
| Assay temperature | 22° C. |
| Read time | 60 mins |
| Std inhibitor | C006* |
| Reader | EnVision multimode plate |
| | reader 2104 |

* the structure of C006 can be found in PCT/U52019/017802.

### Materials:

1. Granzyme B (human lymphocytes) Enzyme: Enzo Lifesciences, Cat #ALX-200-602-0010
2. Substrate (Ac-IETD-AFC): Enzo Lifesciences, Cat #ALX-260-110-M010
3. Standard inhibitor (Compound 20-Isomer3): Synthesized internally in TCG
4. Dimethyl sulfoxide (DMSO): Sigma-Aldrich, Cat #41639
5. HEPES: Gibco, Cat #15630-080
6. Calcium chloride: Sigma-Aldrich, Cat #C-5080
7. Distilled water: Gibco, Cat #15230-162
8. Bovine serum albumin (BSA): Sigma-Aldrich, Cat #A3059
9. Black, clear bottom PDL plate (384 wells): Greiner bio-one, Cat #781946
10. Polypropylene plate (384 wells): Corning, Cat #3657

### Assay Buffer Composition:

TABLE 2
Specific assay buffer compositions

| | |
|---|---|
| Components | Concentration |
| HEPES | 50 mM |
| CaCl2 | 10 mM |

In distilled water
pH adjusted to 7.5 with 5(N) NaOH

### Compound Preparation:

1. DMSO is added to the respective compound vial to make 10 mM Compound stock solution, which is stored at −20° C.
2. 10 mM Compound stock solution is thawed and 1 mM DMSO stock solution is prepared by adding 45 μl of DMSO to 5 μl of 10 mM compound stock solution.
3. 1 mM DMSO stock solution is serially diluted (3.16 fold) by adding 10 μl of 1 mM DMSO stock to 21.6 μl of DMSO and mixed well. 10 μl of the resulting solution is then added to 21.6 μl of DMSO and mixed well. This process is continued to created 11 dilution points for the assay standard inhibitor and 8 dilution points for test compounds in 384 well polypropylene plate.
4. 2 μl of each dilution are dispensed in assay ready plate. 5. Each well is then diluted 25 fold by adding 48 μl of assay buffer to 2 μl of compound in assay ready plate to make Compound working stock.

### Enzyme Preparation:

1. Supplied Granzyme B (human lymphocytes) enzyme is reconstituted to 1 mg/ml (about 31.25 μM) and 1 μl aliquots are kept in −80° C.
2. 1 μl aliquot is diluted to 625 nM by adding 49 μl of assay buffer containing 0.1% BSA and gently mixed.
3. 10 nM enzyme working stock is prepared by adding assay buffer containing 0.1% BSA.

### Substrate Preparation:

1. Supplied substrate (Ac-IETD-AFC) is reconstituted by adding DMSO to make 10 mM stock, aliquoted and stored in −80° C.
2. 4 mM substrate mid stock is prepared by adding DMSO.
3. Substrate working stock i.e. 800 μM is prepared by adding assay buffer.

### Assay Protocol:

1. 10 μl of serially diluted Compound working stock (start dose 40 μM) is added from assay ready plate to assay plate according to the plate map.
2. Positive control (40 μM of standard inhibitor) and negative control (4% DMSO buffer) are added to the respective wells.
3. 20 μl of Enzyme working stock is added to assay plate and gently mixed.
4. The plate is incubated at 22° C. for 30 mins and spun at 130 g for 1 minute.
5. After incubation, 10 μl of Substrate working stock is added to respective wells and mixed (Assay plate is maintained in dark after substrate addition).
6. The plate is incubated at 22° C. for 60 mins and spun at 130 g for 1 minute.
7. Fluorescence read (RFU) (Ex: 400 nm/Em: 505 nm) is taken after 60 mins in EnVision Multimode plate reader.

### Final Assay Concentration:

TABLE 3
Specific final assay concentrations

| | | |
|---|---|---|
| Reagent | Final concentration | Volume added |
| Compound/Std/Controls | Starting from 10 μM, 3.16 | 10 μl |
| | fold serial dilution | |
| Enzyme | 5 nM | 20 μl |
| Substrate | 200 μM | 10 μl |

### Data Analysis:

1. The RFU read is analyzed to calculate percent inhibition by normalizing with positive and negative controls taken as 100% and 0% effect respectively in Microsoft excel.
2. Graph is generated by putting the analyzed data in GraphPad prism 5.0 software to get IC₅₀ value for each compound.

In addition, compounds of Formula I described above are also tested in the metabolic stability assay using liver microsomes, as provided below.

Experimental summary

TABLE 4
Summary of experimental protocol

| | |
|---|---|
| Test system | Metabolic stability |
| Test concentration | 1 μM, n = 2 |
| Liver Microsomes | Human/Rat/Mouse liver |
| | microsomes (0.4 mg/ml) |
| Cofactor | NADPH Regeneration System (NRS) |
| Incubation | 0 and 30 min [OR] 0, 5, 10, 20, 30 |
| | and 60 minutes with NRS at 37° C. |
| Detection | LC-MS/MS |
| Reference standard | Atenolol, Propranolol, Diclofenac, Verapamil |
| Data analysis | % Parent compound remaining/ T-half / Clearance |

### Protocol

Buffer pH7.4: Prepare 1 (M) KH₂PO₄ and 1 (M) K₂HPO₄. Titrate 1(M) K₂HPO₄ with 1 (M) KH₂PO₄ to obtain pH 7.40. Dilute this buffer 10 fold in Water (30 ml buffer+270 ml of water) to obtain 100 mM phosphate buffer. Adjust pH to 7.40±0.02 using 5(N) HCl or 5(N) NaOH.

NADPH Regeneration System (NRS): Prepare a solution containing 13 mM NADP, 33 mM Glucose-6-phosphate, 33 mM MgCl₂ and 4 U/ml Glucose-6-phosphate dehydrogenase in buffer.

Liver Microsome (LM) suspension: Thaw LM vial on ice, then mix 1.0 ml LM (20 mg/ml) with 19 ml buffer [final LM Conc: 1 mg/ml]

LM+NRS suspension: Mix 5.0 ml NRS with 20 ml LM suspension [final LM Conc: 0.8 mg/ml]

System suitability standard: a synthesized compound having Mol wt 686.2 used as System suitability standard. Dissolve this compound in ice-cold acetonitrile to obtain concentration of 0.1 μg/ml and store at 4° C.

Compound Dilution:

Compound Stock: 10 mM in DMSO

Sub stock (100 μM): 4 μl of 10 mM Compound Stock+398 μl Acetonitrile Working plate (2 μM): 10 μl of 100 μM Sub stock+490 μl buffer

### Assay Procedure

Incubate all plastic materials including tips at 37° C. overnight.

Incubate LM suspension and NRS at 37° C. for 15 min before use.

Add 40 μl buffer to the wells of blank plate.

Add 40 μl compound (from working plate) to 0, 5, 10, 20, 30 and 60 min plates.

Initiate reaction by adding 40 μl of LM+NRS suspension in each plate.

Terminate reaction by adding 240 μl ice-cold acetonitrile containing system suitability standard at designated time points. For T=0 add 240 μl ice-cold acetonitrile containing system suitability standard before LM+NRS addition.

Centrifuge (3500 rpm, 20 min and 15° C.) the plates.

Mix 110 μl supernatant with 110 μl water and quantitate amount of Compound in the solution using LC-MS/MS.

### Calculation

% Remaining at t=x min=100×([LC-MS/MS peak area of analyte]_{t=x min})/([LC-MS/MS peak area of analyte]ₜ₌₀ₘᵢₙ)

T_{1/2}=ln 2/−(slope of the ln(% Remaining) vs. time plot)=minutes

$$CLint , app = ln 2 \cdot \frac{1}{T 1 \text{/} 2 ( min )} \cdot \frac{mL incubation}{mg LM protein} \cdot \frac{1000 μl}{mL} = μL \text{/} min \text{/} mg$$

For Scaled CLint & Predicted CL use the relevant physiological parameters, as applicable.

Note:

(1) Source of LM:

Human LM: Corning, Cat #25117

Rat LM: Corning, Cat #452501

Mouse LM: Xenotech, Cat #M1000

(2) Volumes mentioned in the protocol are representative values and may change depending on number of test compounds.

(3) It is assumed that the compounds are chemically stable in the assay system/buffer.

This study does not carry any information on chemical stability or instability.

The metabolic stability assay described above can be performed using different test concentrations, LM Concentrations, or time points.

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever.

Shown in the table below are the structures and named of certain exemplary compounds. These compounds were found to inhibit Granzyme B to various degrees as indicated by their IC₅₀ values included in Table 5 below. Among the symbols in the table, “++++” indicates an IC₅₀ value of 0.001-0.300 μM, “+++” indicates an IC₅₀ value of 0.301-1.000 μM, “++” indicates an IC50 value of 1.001-5 μM, “+” indicates an IC₅₀ value of greater than 5 μM, and “nd” indicates not determined.

TABLE 5
Compounds and IC₅₀ values

| Compound | | | |
|---|---|---|---|
| Number | Structure | Name | IC₅₀ |
| C152 | 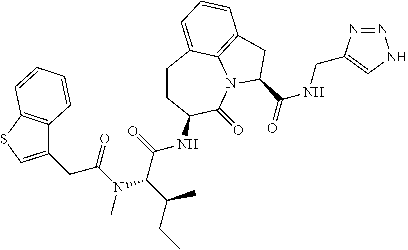 [SMILES: CC[C@H](C)[C@@H](C(=O)N[C@H]1CCc2cccc3c2N(C1=O)[C@H](C(=O)NCc1c[nH]nn1)C3)N(C)C(=O)Cc1csc2ccccc12] | (2S,5S)-5-{(2S,3S)-2-[(2- Benzo[b]thiophen-3-yl-acetyl)- methyl-amino]-3-methyl- pentanoylamino}-4-oxo-1,2,4,5,6,7- hexahydro-azepino[3,2,1-hi]indole-2- carboxylic acid (1H-[1,2,3]triazol-4- ylmethyl)-amide | +++ |
| C155 | 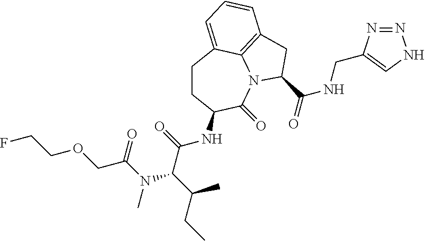 [SMILES: CC[C@H](C)[C@@H](C(=O)N[C@H]1CCc2cccc3c2N(C1=O)[C@H](C(=O)NCc1c[nH]nn1)C3)N(C)C(=O)COCCF] | (2S,5S)-5-((2S,3S)-2-{[2-(2-Fluoro- ethoxy)-acetyl]-methyl-amino}-3- methyl-pentanoylamino)-4-oxo- 1,2,4,5,6,7-hexahydro-azepino[3,2,1- hi]indole-2-carboxylic acid (1H- [1,2,3]triazol-4-ylmethyl)-amide | +++ |
| C176 | 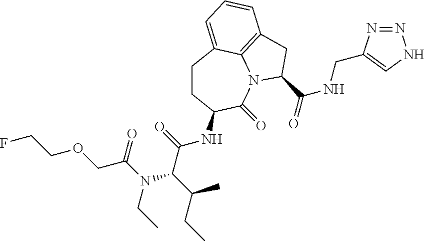 [SMILES: CC[C@H](C)[C@@H](C(=O)N[C@H]1CCc2cccc3c2N(C1=O)[C@H](C(=O)NCc1c[nH]nn1)C3)N(CC)C(=O)COCCF] | (2S,5S)-5-((25,3S)-2-{Ethyl-[2-(2- fluoro-ethoxy)-acetyl]-amino}-3- methyl-pentanoylamino)-4-oxo- 1,2,4,5,6,7-hexahydro-azepino[3,2,1- hi]indole-2-carboxylic acid (1H- [1,2,3]triazol-4-ylmethyl)-amide | +++ |
| C177 | 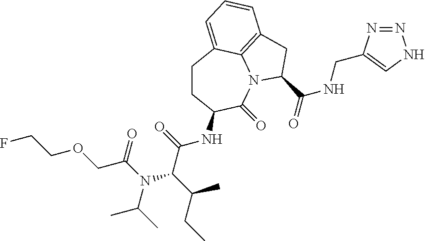 [SMILES: CC[C@H](C)[C@@H](C(=O)N[C@H]1CCc2cccc3c2N(C1=O)[C@H](C(=O)NCc1c[nH]nn1)C3)N(C(=O)COCCF)C(C)C] | (2S,5S)-5-((2S,3S)-2-{[2-(2-Fluoro- ethoxy)-acetyl]-isopropyl-amino}-3- methyl-pentanoylamino)-4-oxo- 1,2,4,5,6,7-hexahydro-azepino[3,2,1- hi]indole-2-carboxylic acid (1H- [1,2,3]triazol-4-ylmethyl)-amide | ++ |
| C178 | 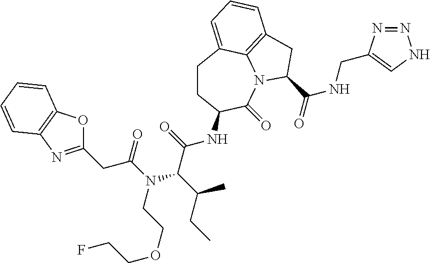 [SMILES: CC[C@H](C)[C@@H](C(=O)N[C@H]1CCc2cccc3c2N(C1=O)[C@H](C(=O)NCc1c[nH]nn1)C3)N(CCOCCF)C(=O)Cc1nc2ccccc2o1] | (2S,5S)-5-((2S,3S)-2-{(2- Benzooxazol-2-yl-acetyl)-[2-(2- fluoro-ethoxy)-ethyl]-amino}-3- methyl-pentanoylamino)-4-oxo- 1,2,4,5,6,7-hexahydro-azepino[3,2,1- hi]indole-2-carboxylic acid (1H- [1,2,3]triazol-4-ylmethyl)-amide | +++ |
| C179 | 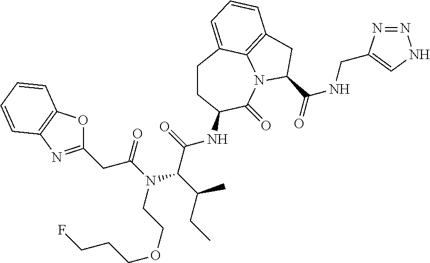 [SMILES: CC[C@H](C)[C@@H](C(=O)N[C@H]1CCc2cccc3c2N(C1=O)[C@H](C(=O)NCc1c[nH]nn1)C3)N(CCOCCCF)C(=O)Cc1nc2ccccc2o1] | (2S,5S)-5-((2S,3S)-2-{(2- Benzooxazol-2-yl-acetyl)-[2-(3- fluoro-propoxy)-ethyl]-amino}-3- methyl-pentanoylamino)-4-oxo- 1,2,4,5,6,7-hexahydro-azepino[3,2,1- hi]indole-2-carboxylic acid (1H- [1,2,3]triazol-4-ylmethyl)-amide | +++ |
| C184 | 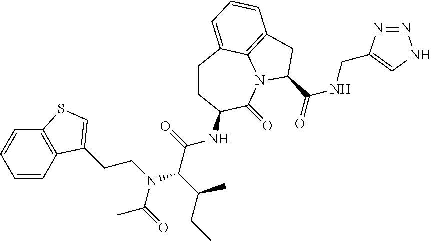 [SMILES: CC[C@H](C)[C@@H](C(=O)N[C@H]1CCc2cccc3c2N(C1=O)[C@H](C(=O)NCc1c[nH]nn1)C3)N(CCc1csc2ccccc12)C(C)=O] | (2S,5S)-5-{(2S,3S)-2-[Acetyl-(2- benzo[b]thiophen-3-yl-ethyl)-amino]- 3-methyl-pentanoylamino}-4-oxo- 1,2,4,5,6,7-hexahydro-azepino[3,2,1- hi]indole-2-carboxylic acid (1H- [1,2,3]triazol-4-ylmethyl)-amide | + |
| C190 | 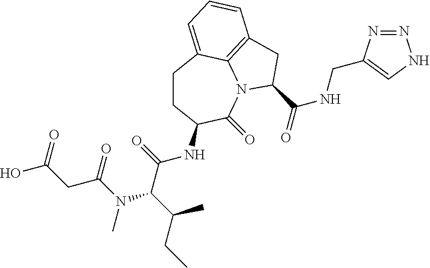 [SMILES: CC[C@H](C)[C@@H](C(=O)N[C@H]1CCc2cccc3c2N(C1=O)[C@H](C(=O)NCc1c[nH]nn1)C3)N(C)C(=O)CC(=O)O] | N-Methyl-N-((1S,2S)-2-methyl-1- {(2S,5S)-4-oxo-2-[(1H-[1,2,3]triazol- 4-ylmethyl)-carbamoyl]-1,2,4,5,6,7- hexahydro-azepino[3,2,1-hi]indol-5- ylcarbamoyl}-butyl)-malonamic acid | +++ |
| C191 | 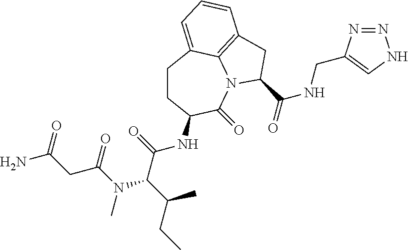 [SMILES: CC[C@H](C)[C@@H](C(=O)N[C@H]1CCc2cccc3c2N(C1=O)[C@H](C(=O)NCc1c[nH]nn1)C3)N(C)C(=O)CC(N)=O] | N-Methyl-N-((1S,2S)-2-methyl-1- {(2S,5S)-4-oxo-2-{(1H-[1,2,3]triazol- 4-ylmethyl)-carbamoyl]-1,2,4,5,6,7- hexahydro-azepino[3,2,1-hi]indol-5- ylcarbamoyl}-butyl)-malonamide | +++ |
| C192 | 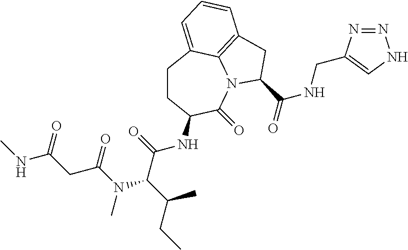 [SMILES: CC[C@H](C)[C@@H](C(=O)N[C@H]1CCc2cccc3c2N(C1=O)[C@H](C(=O)NCc1c[nH]nn1)C3)N(C)C(=O)CC(=O)NC] | N,N′-Dimethyl-N-((1S,2S)-2-methyl- 1-{(2S,5S)-4-oxo-2-[(1H- [1,2,3[triazol-4-ylmethyl)- carbamoyl]-1,2,4,5,6,7-hexahydro- azepino[3,2,1-hi]indol-5- ylcarbamoyl}-butyl)-malonamide | +++ |
| C193 | 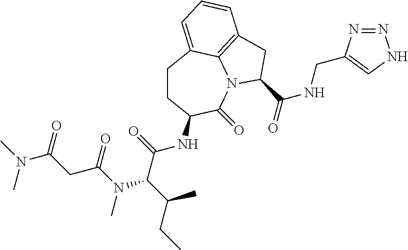 [SMILES: CC[C@H](C)[C@@H](C(=O)N[C@H]1CCc2cccc3c2N(C1=O)[C@H](C(=O)NCc1c[nH]nn1)C3)N(C)C(=O)CC(=O)N(C)C] | N,N,N′-Trimethyl-N′-((lS,2S)-2- methyl-1-{(2S,5S)-4-oxo-2-[(1H- [1,2,3]triazol-4-ylmethyl)- carbamoyl]-1,2,4,5,6,7-hexahydro- azepino[3,2,1-hi]indol-5- ylcarbamoyl}-butyl)-malonamide | +++ |
| C194 | 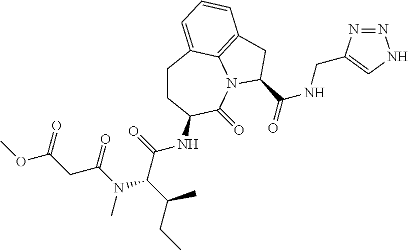 [SMILES: CC[C@H](C)[C@@H](C(=O)N[C@H]1CCc2cccc3c2N(C1=O)[C@H](C(=O)NCc1c[nH]nn1)C3)N(C)C(=O)CC(=O)OC] | N-Methyl-N-((1S,2S)-2-methyl-1- {(2S,5S)-4-oxo-2-[(1H-[1,2,3]triazol- 4-ylmethyl)-carbamoyl]-1,2,4,5,6,7- hexahydro-azepino[3,2,1-hi]indol-5- ylcarbamoyl}-butyl)-malonamic acid methyl ester | +++ |
| C198 | 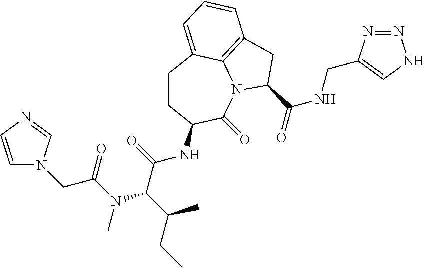 [SMILES: CC[C@H](C)[C@@H](C(=O)N[C@H]1CCc2cccc3c2N(C1=O)[C@H](C(=O)NCc1c[nH]nn1)C3)N(C)C(=O)Cn1ccnc1] | (2S,5S)-5-{(2S,3S)-2-[(2-Imidazo1-1- yl-acetyl)-methyl-amino]-3-methyl- pentanoylamino}-4-oxo-1,2,4,5,6,7- hexahydro-azepino[3,2,1-hi]indole-2- carboxylic acid (1H-[1,2,3]triazol-4- ylmethyl)-amide | +++ |
| C203 | 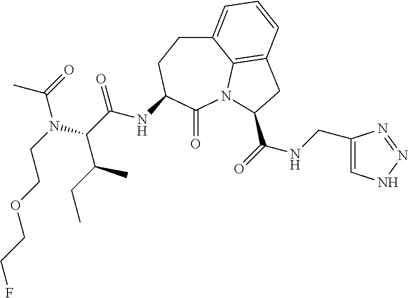 [SMILES: CC[C@H](C)[C@@H](C(=O)N[C@H]1CCc2cccc3c2N(C1=O)[C@H](C(=O)NCc1c[nH]nn1)C3)N(CCOCCF)C(C)=O] | (2S,5S)-5-((2S,3S)-2-{Acetyl-[2-(2- fluoro-ethoxy)-ethyl]aminol -3- methyl-pentanoylamino)-4-oxo- 1,2,4,5,6,7-hexahydro-azepino[3,2,1- hi]indole-2-carboxylic acid (1H- [1,2,3[triazol-4-ylmethyl)-amide | +++ |
| C195 | 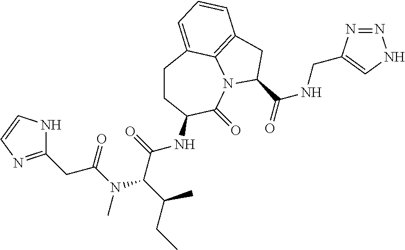 [SMILES: CC[C@H](C)[C@@H](C(=O)N[C@H]1CCc2cccc3c2N(C1=O)[C@H](C(=O)NCc1c[nH]nn1)C3)N(C)C(=O)Cc1ncc[nH]1] | (2S,5S)-5-{(2S,3S)-2-[(2-1H- Imidazo1-2-yl-acetyl)-methyl-amino]- 3-methyl-pentanoylamino}-4-oxo- 1,2,4,5,6,7-hexahydro-azepino[3,2,1- hi]indole-2-carboxylic acid (1H- [1,2,3[triazol-4-ylmethyl)-amide | nd |
| C197 | 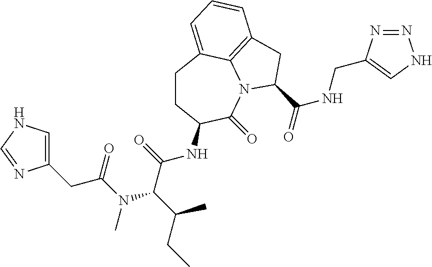 [SMILES: CC[C@H](C)[C@@H](C(=O)N[C@H]1CCc2cccc3c2N(C1=O)[C@H](C(=O)NCc1c[nH]nn1)C3)N(C)C(=O)Cc1c[nH]cn1] | (2S,5S)-5-{(2S,3S)-2-(2-1H- Imidazo1-4-yl-acetyl)-methyl-amino]- 3-methyl-pentanoylamino}-4-oxo- 1,2,4,5,6,7-hexahydro-azepino[3,2,1- hi]indole-2-carboxylic acid (1H- [1,2,3]triazol-4-ylmethyl)-amide | +++ |
| C214 | 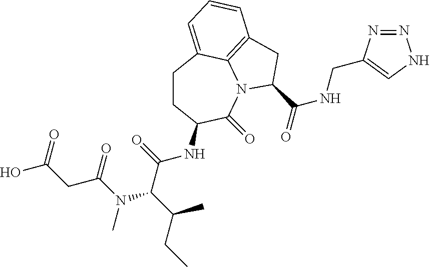 [SMILES: CC[C@H](C)[C@@H](C(=O)N[C@H]1CCc2cccc3c2N(C1=O)[C@H](C(=O)NCc1c[nH]nn1)C3)N(C)C(=O)CC(=O)O] | N-Methyl-N-((1S,2S)-2-methyl-1- {(1S,8S)-9-oxo-1-[(1H-[1,2,3]triazol- 4-y lmethyl)-carbamoyl]-1,2,8,9- tetrahydro-7H-6-oxa-9a-aza- benzo[cd]azu1en-8-ylcarbamoyl}- butyl)-malonamic acid | +++ |
| C230 | 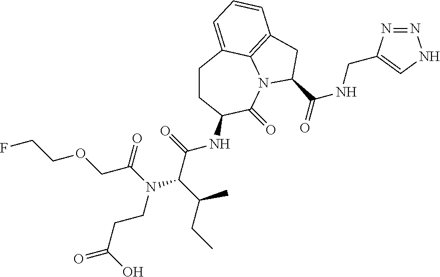 [SMILES: CC[C@H](C)[C@@H](C(=O)N[C@H]1CCc2cccc3c2N(C1=O)[C@H](C(=O)NCc1c[nH]nn1)C3)N(CCC(=O)O)C(=O)COCCF] | 3-[[2-(2-Fluoro-ethoxy)-acetyl]- ((1S,2S)-2-methyl-1-{(2S,5S)-4-oxo- 2-[(1H-[1,2,3]triazol-4-ylmethyl)- carbamoyl]-1,2,4,5,6,7-hexahydro- azepino[3,2,1-hi]indol-5- ylcarbamoyl}-butyl)-amino]- propionic acid | +++ |

Described below are the procedures used to synthesize the above-descried exemplary compounds. The synthesis of certain intermediates, such as the Central Scaffold in Scheme-18 below, can be found in PCT/US2019/017802.

### Example 1: Synthesis of Compounds Wherein X is CH₂

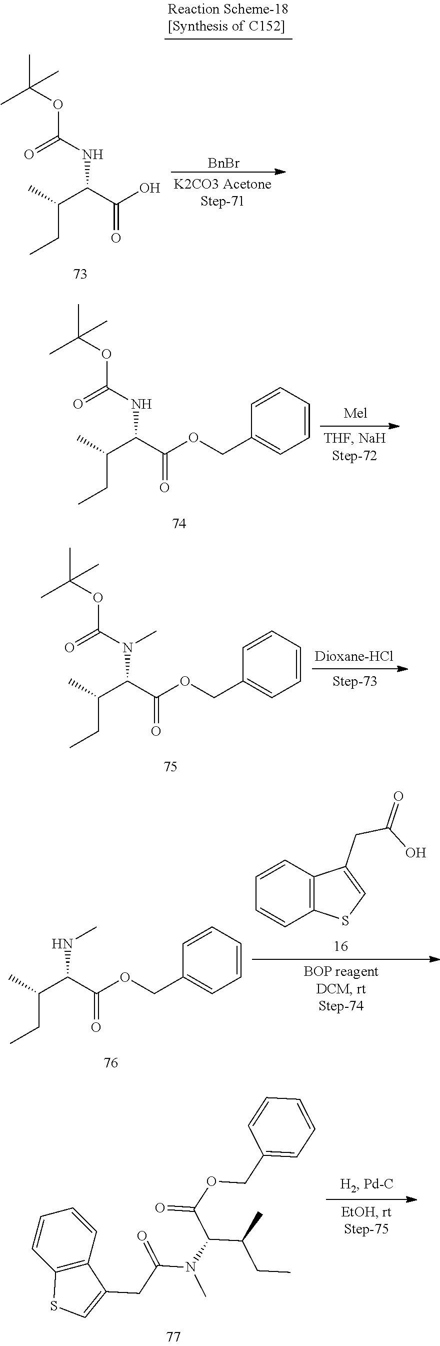

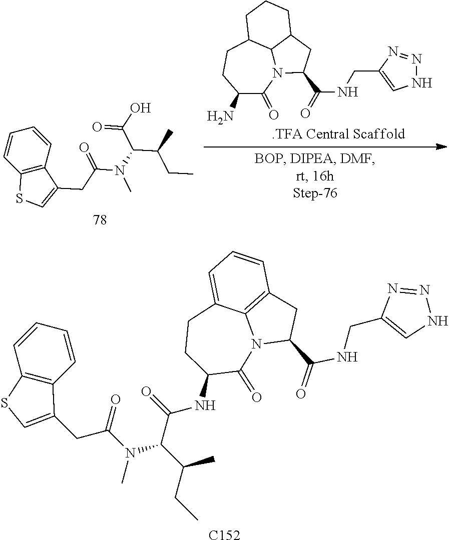 [SMILES: CC[C@H](C)[C@@H](C(=O)O)N(C)C(=O)Cc1csc2ccccc12 ; N[C@H]1CCC2CCCC3C[C@@H](C(=O)NCc4c[nH]nn4)N(C1=O)C23 ; CC[C@H](C)[C@@H](C(=O)N[C@H]1CCc2cccc3c2N(C1=O)[C@H](C(=O)NCc1c[nH]nn1)C3)N(C)C(=O)Cc1csc2ccccc12 ; "C152"]

Synthesis of (2S, 3S)-2-tert-Butoxycarbonylamino-3-methyl-pentanoic acid benzyl ester [Scheme-18, Step-71]: To a stirred solution of compound 73 (5.00 g, 21.6 mmol) in acetone (10 mL), was added K2CO3 (7.50 g, 54.0 mmol) and benzyl bromide (5.14 mL, 43.2 mmol) respectively at room temperature under argon atmosphere and allowed to stir at 60° C. for 16 h. After completion [confirmed by TLC (10% EtOAc-Hexane, Rf-0.5) and LCMS] reaction mixture was concentrated under reduced pressure and partitioned between water (100 mL and EtOAc (3×50 mL). Organic layer was separated, dried (MgSO₄) and concentrated under reduced pressure. The resultant crude was purified by column chromatography (eluent: 3% EtOAc-hexane, SiO₂) to provide compound 74 (4.1 g, 59%) as pale yellow liquid. Mass [ESI]: m/z 321.41 [M⁺+1].

Synthesis of (2S,3S)-2-(tert-Butoxycarbonyl-methyl-amino)-3-methyl-pentanoic acid benzyl ester [Scheme-18, Step-72]: To a stirred solution of compound 74 (500 mg, 1.06 mmol) in THF (4 mL) at 0° C. was added NaH (131 mg, 3.27 mmol) portion-wise under nitrogen atmosphere and allowed to stir for 10 minutes. To the resultant reaction mixture was added MeI (0.306 mL, 4.91 mmol), and allowed to stir for 2 hour at room temperature. After completion [confirmed by TLC (10% EtOAc-Hexane, R_{f}-0.5) and LC-MS] reaction mixture was concentrated under reduced pressure, resultant crude was partitioned between EtOAc (75 mL) and water (50 mL). Organic layer was separated, washed with brine (30 mL), dried (MgSO₄) and concentrated. Crude residue was purified by column chromatography (eluent: 3-5% EtOAc-Hexane, SiO₂) to afford compound 75 (0.20 g, 36%) as pale yellow liquid. [¹H NMR complies].

Synthesis of (2S,3S)-3-Methyl-2-methylamino-pentanoic acid benzyl ester [Scheme-18, Step-73]: To a stirred solution of compound 75 (0.10 g, 0.31 mmol) in 2 mL Dioxane, was added 1 mL of 4 (N) HCl in dioxane at room temperature under inert atmosphere and allowed to stir for 1 h at room temperature. After completion [confirmed by LC-MS and TLC (10% EtOAc-Hexane, Rf-0.1)]; reaction mixture was concentrated under reduced pressure to provide HCl salt of compound 76 (80 mg, yield-quatitative) as off white solid. Mass [ESI]: m/z 235.32 [M⁺+1].

Synthesis of (2S,3S)-2-[(2-Benzo[b]thiophen-3-yl-acetyl)-methyl-amino]-3-methyl-pentanoic acid benzyl ester [Scheme-18, Step-74]: To a stirred solution of compound 16 (500 mg, 1.95 mmol), compound 76 (HCl salt) (375 mg, 1.95 mmol) in DCM (10 mL) was added DIPEA (1.70 mL, 9.77 mmol) drop-wise for 10 min under inert atmosphere. To the resultant mixture BOP (1.73 g, 3.91 mmol) was added and allowed to stir at room temperature for 16 h. After completion [confirmed by LC-MS and TLC (30% EtOAc-Hexane, Rf-0.7)]; reaction mixture was partitioned between water (60 mL) and DCM (3×50 mL]. Organic layer was separated, washed with brine (30 m), dried (MgSO₄) and concentrated. Resultant crude was purified by column chromatography (eluent: 6% EtOAc-Hexane, SiO₂) to provide compound 77 (0.73 g, 91%) as yellow gum. Mass [ESI]: m/z 409.54 [M⁺+1].

Synthesis of (2S,3S)-2-[(2-Benzo[b]thiophen-3-yl-acetyl)-methyl-amino]-3-methyl-pentanoic acid [Scheme-18, Step-75]: To a stirred solution of compound 77 (730 mg, 1.78 mmol) in EtOH (8 mL), 500 mg of 10% by weight Pd/C (wet) powder was added and allowed to stir under ordinary hydrogen pressure (balloon) for 16 h at room temperature. After completion [confirmed by TLC (30% EtOAc-Hexane, Rf-0.1) and LC-MS], reaction mixture was filtered through a celite bed, concentrated under reduced pressure. Resultant crude was triturated with pentane to afford compound 78 (0.25 g, 44%) as white solid.

Synthesis of (2S, 5S)-5-{(2S, 3S)-2-[(2-Benzo[b]thiophen-3-yl-acetyl)-methyl-amino]-3-methyl-pentanoylamino}-4-oxo-1,2,4,5,6,7-hexahydro-azepino[3,2,1-hi]indole-2-carboxylicacid(1H-[1,2,3]triazol-4-ylmethyl)-amide (C152) [Scheme-18, Step-76]: To a stirred solution of compound 78 (0.12 g, 0.38 mmol) and central scaffold (0.15 g, 0.34 mmol) in DMF (2.0 mL) was added DIPEA (0.30 mL, 1.7 mmol), followed by BOP reagent (0.30 g, 0.68 mmol) at rt, under argon atmosphere, and continued at the same temperature for 16 h. After completion [confirmed by LC-MS], crude reaction mixture was submitted for reverse phase prep HPLC purification to afford 20 mg of faster eluting as major isomer [Isomer1] of C152 as white solid. Mass [ESI]: m/z 627.77 [M⁺+1].

Synthesis of (2S, 5S)-5-((2S, 3S)-2-{[2-(2-Fluoro-ethoxy)-acetyl]-methyl-amino}-3-methyl-pentanoylamino)-4-oxo-1,2,4,5,6,7-hexahydro-azepino[3,2,1-hi]indole-2-carboxylic acid (1H-[1,2,3]triazol-4-ylmethyl)-amide (C155): This compound was synthesized following the same protocol as for C152. Prep HPLC purification afforded 27 mg of faster eluting as major isomer [Isomer1] of C155 as white solid. Mass [ESI]: m/z 557.63 [M⁺+1].

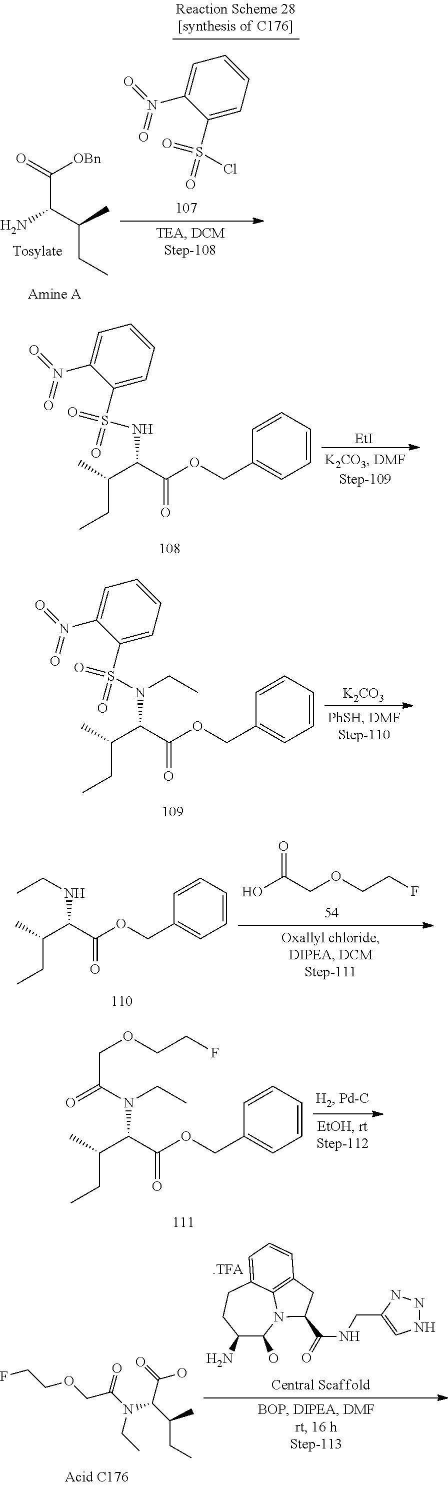

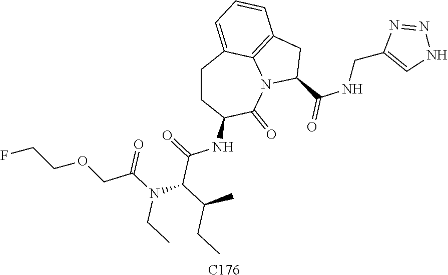 [SMILES: CC[C@H](C)[C@@H](C(=O)N[C@H]1CCc2cccc3c2N(C1=O)[C@H](C(=O)NCc1c[nH]nn1)C3)N(CC)C(=O)COCCF ; "C176"]

Synthesis of (2S,3S)-3-Methyl-2-(2-nitro-benzenesulfonylamino)-pentanoic acid benzyl ester [Scheme-28, Step-108]: To a stirred solution of Amine A (5.0 gm, 12 mmol) in DCM (20 mL) was added TEA (10.6 mL, 76.2 mmol) followed by portion-wise addition of 107 (3.38 gm, 15.2 mmol) and resultant reaction mixture was allowed to stir at room temperature for 16 h. Then reaction mixture was concentrated under reduced pressure. Resultant crude was partitioned between EtOAc and 1 (N) aqueous KHSO₄ solution. Organic layer was separated, dried (MgSO₄) and concentrated under reduced pressure. Crude residue thus obtained was purified by column chromatography using silica gel 100-200 mesh as absorbent under gradient elution of 15-20% EtOAc-hexane to afford compound 108 (3.4 gm) as colorless semi solid. Mass [ESI]: m/z 406.45 [M⁺+1].

Synthesis of (2S,3S)-2-[Ethyl-(2-nitro-benzenesulfonyl)-amino]-3-methyl-pentanoic acid benzyl ester [Scheme-28, Step-109]: To a stirred solution of compound 108 (1.0 gm, 2.46 mmol) in DMF (5.0 mL) was added K2CO3 (680 mg, 4.92 mmol) followed by addition of EtI (0.8 mL, 9.8 mmol) and resultant reaction mixture was allowed to stir at room temperature for 2.5 h. After completion (monitored with TLC-20% EtOAc-hexane, Rf-0.4 & LC-MS), resultant crude was partitioned between EtOAc and cold water. Organic layer was separated, dried (MgSO₄) and concentrated under reduced pressure to afford compound 109 [1.1 gm, crude compound] as yellow liquid. Mass [ESI]: m/z 434.50 [M⁺+1].

Synthesis of (2S,3S)-2-Ethylamino-3-methyl-pentanoic acid benzyl ester [Scheme-28, Step-110]: To a stirred solution of compound 109 (1.50 gm, 3.45 mmol) in DMF (5.0 mL) was added K2CO3 (1.24 gm, 8.97 mmol) followed by addition of PhSH (0.35 mL, 3.45 mmol) and resultant reaction mixture was allowed to stir at room temperature for 1.5 h. After completion (monitored with TLC, 5% MeOH in DCM, Rf-0.3 & LC-MS) resultant crude was partitioned between EtOAc and cold water. Organic layer was separated, dried (MgSO₄) and concentrated under reduced pressure. Resultant crude was purified by column chromatography using silica gel 100-200 mesh and under gradient elution of 0-5% MeOH-DCM to afford compound 110 [800 mg, 92.1%] as sticky yellow liquid. Mass [ESI]: m/z 249.35 [M⁺+1].

Synthesis of (2S,3S)-2-{Ethyl-[2-(2-fluoro-ethoxy)-acetyl]-amino}-3-methyl-pentanoic acid benzyl ester [Scheme-28, Step-111]: To a stirred solution of compound 54 (200 mg, 1.64 mmol) in DCM (5.0 mL) was added oxalyl chloride (0.28 mL, 3.27 mmol) followed by addition of catalytic amount of DMF and the resultant reaction mixture was allowed to stir at room temperature for 30 minutes. Then reaction mixture was concentrated under reduced pressure and resultant crude acid chloride was diluted with DCM (5.0 mL) followed by addition of DIPEA (1.40 mL, 8.19 mmol), compound 110 (408 mg, 1.63 mmol) and allowed to stir at room temperature for 3 h. Then reaction mixture was partitioned between EtOAc and water. Organic layer was separated, dried (MgSO₄) and concentrated under reduced pressure. Crude residue thus obtained was purified by column chromatography (eluent: 15-20% EtOAc-hexane, absorbent SiO₂) to afford compound 111 [130 mg, 22%] as light brown sticky liquid. Mass [ESI]: m/z 353.4 [M⁺+1].

Synthesis of (2S,3S)-2-{Ethyl-[2-(2-fluoro-ethoxy)-acetyl]-amino}-3-methyl-pentanoic acid [Scheme-28, Step-112]: To a stirred solution of compound 111 (130 mg, 0.37 mmol) in EtOH (5.0 mL) was added of 10% by weight Pd/C (wet) powder (60 mg) and degassed under argon atmosphere for 10 minutes. The resultant suspension was allowed to stir under ordinary hydrogen pressure (balloon) at room temperature for 16 h. After completion reaction mixture was filtered through celite bed, filtrate part was concentrated under reduced pressure to afford Acid C176 (90 mg, 92%) as light brown liquid. Mass [ESI]: m/z 263.3 [M⁺+1].

Synthesis of (2S, 5S)-5-((2S, 3S)-2-{Ethyl-[2-(2-fluoro-ethoxy)-acetyl]-amino}-3-methyl-pentanoylamino)-4-oxo-1, 2, 4, 5, 6, 7-hexahydro-azepino [3,2,1-hi]indole-2-carboxylic acid (1H-[1,2,3]triazol-4-ylmethyl)-amide [Scheme-1, Step-5]: To a stirred solution of central scaffold (150 mg, 0.34 mmol) and Acid C176 (90.0 mg, 0.34 mmol) in DMF (2.0 mL) was added DIPEA (0.3 mL, 1.7 mmol), followed by the addition of BOP reagent (302 mg, 0.68 mmol) and resultant reaction mixture was allowed to stir at room temperature for 16 h. After completion [monitored with LC-MS], crude reaction mixture was submitted for reverse phase prep HPLC purification to afford 5 mg of faster eluting isomer [Isomer 1] of C176 as white solid. Mass [ESI]: m/z 571.64 [M⁺+1].

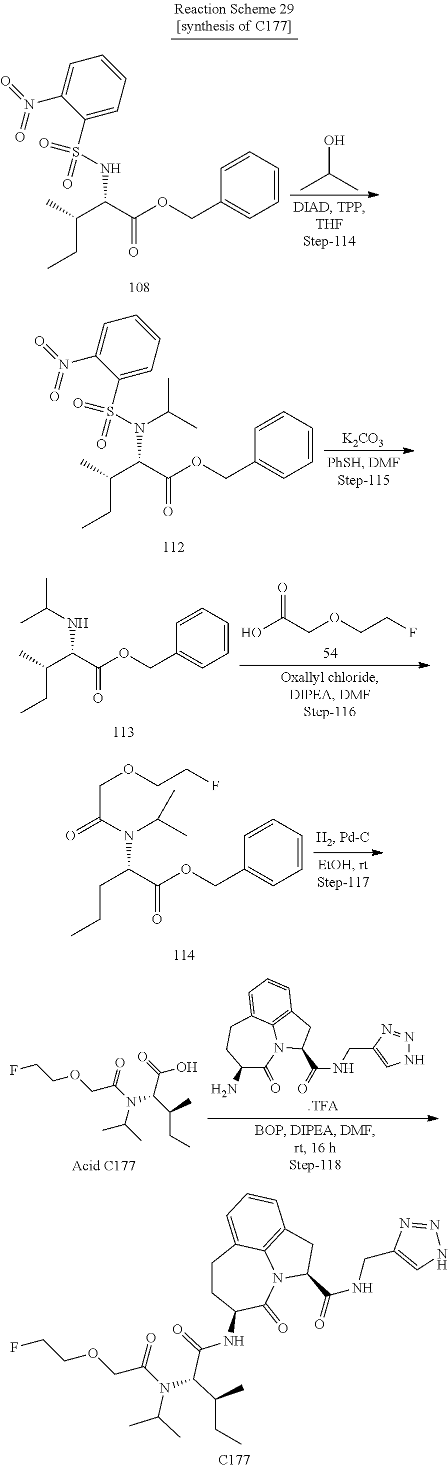

Synthesis of (2S,3S)-2-[Isopropyl-(2-nitro-benzenesulfonyl)-amino]-3-methyl-pentanoic acid benzyl ester [Scheme 29, Step-114]: To a stirred solution of compound 108 (1.70 gm, 4.18 mmol) and iso propanol (3.20 mL, 41.8 mmol) in THF (10 mL) was added PPh₃ (2.19 gm, 8.36 mmol) followed by addition of DIAD (1.60 mL, 8.36 mmol) at 0° C. and the resultant reaction mixture was allowed to stir at room temperature for 16 h. After LC-MS analysis reaction mixture was partitioned between water and water. Organic part was separated, dried over Na₂SO₄ and concentrated under reduced pressure. Crude residue thus obtained was purified by column chromatography (eluent: 2-8% EtOAc-Hexane) to afford desired compound 112 (1.32 gm, 70.3%) as off white gum. Mass [ESI]: m/z 448.53 [M⁺+1]

Synthesis of (2S,3S)-2-Isopropylamino-3-methyl-pentanoic acid benzyl ester [Scheme 29, Step-115]: To a stirred solution of compound 112 (1.60 gm, 7.54 mmol) in DMF (5.0 mL) was added K₂CO₃ (1.00 gm, 7.65 mmol) followed by PhSH (0.60 mL, 5.88 mmol) at room temperature the resultant reaction mixture was allowed to stir at room temperature for 1.5 h. After completion of the reaction (confirmed by TLC), the reaction mixture was quenched with ice cold water, extracted with EtOAc, washed with NaHCO₃ solution and brine, dried over Na₂SO₄ and evaporated under reduced pressure. Resultant crude was purified by combiflash chromatography (Eluent: 0-20% EtOAc-Hexane) to afford compound 113 (830 mg, 90.1%) as pale brown gummy liquid. Mass [ESI]: m/z 263.38 [M⁺+1]

Synthesis of (2S,3S)-2-{[2-(2-Fluoro-ethoxy)-acetyl]-isopropyl-amino}-3-methyl-pentanoic acid benzyl ester [Scheme 29, Step-116]: To a stirred solution of compound 113 (500 mg, 4.10 mmol) in DCM (3.0 mL) was added oxalyl chloride (0.35 mL, 4.10 mmol) and 1 drop of DMF at room temperature under argon atmosphere and allowed to stir for 1 h. Then reaction mixture was concentrated under reduced pressure and inert atmosphere. To the resultant mixture was added DCM (4.0 mL), followed by drop-wise addition of DIPEA (3.57 mL, 20.5 mmol), and compound 54 (1.10 gm, 4.10 mmol), and stirred at room temperature under inert atmosphere for 16 h. On completion of the reaction (confirmed by TLC and LCMS), the mixture was extracted by DCM, washed with water, brine, dried (Na₂SO₄) and concentrated. The resultant crude was purified by Prep TLC (eluent: 20% EtOAc-Hexane), to afford compound 114 (150 mg, 10.2%) as pale yellow gum. Mass [ESI]: m/z 367.46 [M⁺+1]

Synthesis of (2S,3S)-2-{[2-(2-Fluoro-ethoxy)-acetyl]-isopropyl-amino}-3-methyl-pentanoic acid [Scheme 29, Step-117]: To a stirred solution of compound 114 (150 mg, 0.41 mmol) in EtOH (4.0 mL), 10% Pd/C (wet) (50 mg) powder was added and degassed under argon atmosphere for 10 minutes. The resultant reaction mixture was allowed to stir under H₂ balloon pressure for 16 h at room temperature. Then reaction mixture was filtered through celite bed and filtrate part was concentrated under reduced pressure to afford Acid C177 (100 mg, 88.1%) as off white gummy liquid. Mass [ESI]: m/z 277.34 [M⁺+1]

Synthesis of (2S, 5S)-5-((2S, 3S)-2-{[2-(2-Fluoro-ethoxy)-acetyl]-isopropyl-amino}-3-methyl-pentanoylamino)-4-oxo-1,2,4,5,6,7-hexahydro-azepino[3,2,1-hi]indole-2-carboxylic acid (1H-[1,2,3]triazol-4-ylmethyl)-amide [Scheme-29, Step-118]: To a stirred solution of central scaffold (150 mg, 0.34 mmol) and Acid C177 (94.5 mg, 0.34 mmol) in DMF (2.0 mL) was added DIPEA (0.30 mL, 1.70 mmol), followed by the addition of BOP reagent (301 mg, 0.68 mmol) and resultant reaction mixture was allowed to stir at room temperature for 16 h. After completion [monitored with LC-MS], crude reaction mixture was submitted for reverse phase prep HPLC purification to afford 8.5 mg of faster eluting isomer [Isomer 1] of C177 as white solid. Mass [ESI]: m/z 585.68 [M⁺+1].

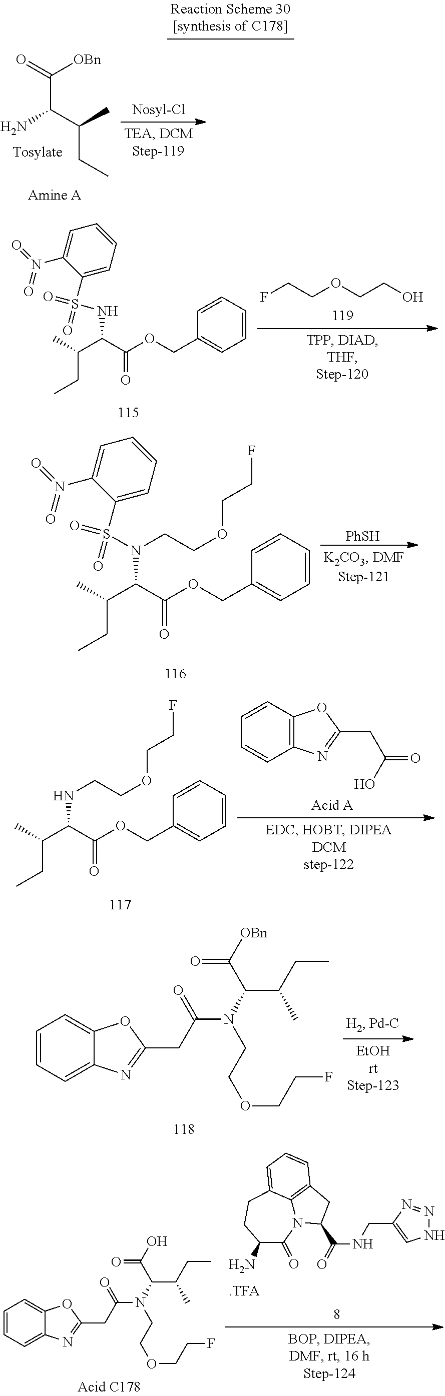

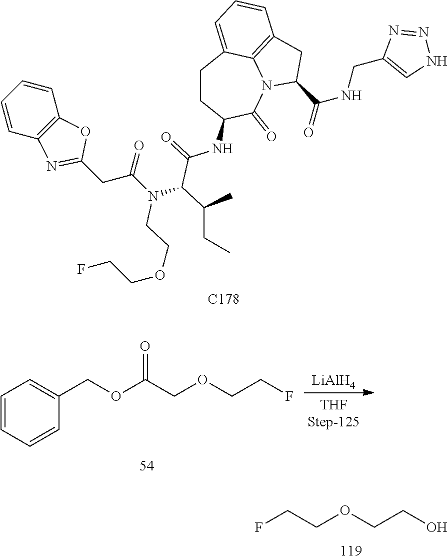

Synthesis of (2S,3S)-3-Methyl-2-(2-nitro-benzenesulfonylamino)-pentanoic acid benzyl ester [Scheme 30, Step-119]: To a stirred solution of Amine A (5.00 gm, 12.7 mmol) in DCM (50 mL) was added Et₃N (10.6 mL, 76.2 mmol) followed by slow addition of Nosyl Chloride (3.38 gm, 15.2 mmol) at room temperature and allowed to stir for 16 h. After completion (monitored by LC-MS), reaction mixture was evaporated to dryness, diluted with EtOAc, washed with 1(N) KHSO₄ and brine. Then reaction mixture was dried over sodium sulphate and concentrated under reduced pressure, resultant crude was purified by column chromatography (eluent: 15-20% EtOAc-Hexane) to afford compound 115 (2.7 g, 52%) as colorless semi-solid. Mass [ESI]: m/z 406.46 [M⁺+1].

Synthesis of (2S, 3S)-2-[[2-(2-Fluoro-ethoxy)-ethyl]-(2-nitro-benzenesulfonyl)-amino]-3-methyl-pentanoic acid benzyl ester [Scheme 30, Step-120]: To a stirred solution of compound 115 (1.36 μm, 3.35 mmol) and compound 119 (724 mg, 6.69 mmol) in THF (6.0 mL) was added TPP (1.76 gm, 6.69 mmol) followed by DIAD (1.32 mL, 6.69 mmol) drop wise under ice cold condition and allowed to stir for 4 h at room temperature. After completion, (confirmed by LC-MS) reaction mixture was diluted with ethyl acetate and washed with excess water followed by brine. The organic part was concentrated under reduce pressure and purified by column chromatography [Absorbent SiO₂, eluting solvent 10-18% EtOAc-Hexane] to afford compound 116 contaminated with some UV inactive impurity. This crude was further purified by column chromatography using 0.1% IPA in DCM [To get rid of the KMnO₄ active impurity] as eluting solvent to afford the compound 116 (1.35 gm, 81.2%) as gummy liquid. Mass [ESI]: m/z 496.56 [M⁺+1]

Synthesis of (2S,3S)-2-[2-(2-Fluoro-ethoxy)-ethylamino]-3-methyl-pentanoic acid benzyl ester [Scheme 30, Step-121]: To a stirred solution of compound 116 (1.50 gm, 3.02 mmol) in DMF (10 mL), was added K₂CO₃ (1.09 gm, 7.85 mmol) followed by the addition of PhSH (0.31 mL, 3.02 mmol) and the resultant reaction mixture was allowed to stir at room temperature for 1.5 h. After completion (confirmed by TLC), reaction mixture was quenched with ice cold water, extracted with ethyl acetate, washed with NaHCO₃ solution and brine, dried over Na₂SO₄ and evaporated under reduced pressure. Resultant crude was purified by combiflash chromatography (Eluent: 0-20% EtOAc-hexane) to provide compound 117 (750 mg, 79.1%) as colorless liquid. Mass [ESI]: m/z 311.40 [M⁺+1].

Synthesis of (2S,3S)-2-{(2-Benzooxazol-2-yl-acetyl)-[2-(2-fluoro-ethoxy)-ethyl]-amino}-3-methyl-pentanoic acid benzyl ester [Scheme 30, Step-122]: To a stirred solution of compound 117 (200 mg, 0.64 mmol) and Acid A (129 mg, 0.64 mmol) in DCM (20 mL) were added DIPEA (0.58 mL, 3.21 mmol) at 0° C. Finally EDC.HCl (160 mg, 0.84 mmol) and HOBT (130 mg, 0.96 mmol) were added in one portion at ice cold condition and the reaction mixture was stirred for 16 h at room temperature. After completion (monitored by LC-MS and TLC), reaction mixture was quenched with water and extracted with EtOAc. Organic layer was dried over Na₂SO₄ and concentrated under reduced pressure. Resultant crude was purified by column chromatography (eluent: 0-20% EtOAc-hexane) to afford compound 118 (150 mg, 49.3%) as gummy liquid. Mass [ESI]: m/z 470.55 [M⁺+1]

Synthesis of (2S,3S)-2-{(2-Benzooxazol-2-yl-acetyl)-[2-(2-fluoro-ethoxy)-ethyl]-amino}-3-methyl-pentanoic acid [Scheme 30, Step-123]: To a solution of Compound 118 (150 mg, 0.32 mmol) dissolved in EtOH (15 mL) was added Pd—C (100 mg, wet 10%) and resultant suspension was degassed with nitrogen for 10 minutes. Then reaction mixture was stirred for 4 h at room temperature under hydrogen balloon pressure. After completion (confirmed by TLC), reaction mixture was filtered through celite and the filtrate was concentrated to provide Acid C178 (140 mg, 98.3%) as gummy liquid. Mass [ESI]: m/z 380.42 [M⁺+1]

Synthesis of 2-(2-Fluoro-ethoxy)-ethanol [Scheme 30, Step-125]: To a suspension of LiAlH₄ (941 mg, 24.7 mmol) in Et₂O (50 mL) was added solution of Compound 53 (1.75 gm, 8.25 mmol) in Et₂O (10 mL) at 0° C. and allowed to stir at room temperature for 3 h. After completion (monitored with TLC) reaction mixture was slowly quenched with saturated aqueous solution of sodium sulphate under ice cold condition. Filtrate was concentrated under reduced pressure and resultant crude was purified by column chromatography [absorbent-SiO₂, 20-35% EtOAc-hexane] to afford compound 119 [250 mg, 28.01%] as pale yellow liquid. Mass [ESI]: m/z 108.11 [M⁺+1]

Synthesis of (2S,5S)-5-((2S,3S)-2-{(2-Benzooxazol-2-yl-acetyl)-[2-(2-fluoro-ethoxy)-ethyl]-amino}-3-methyl-pentanoylamino)-4-oxo-1,2,4,5,6,7-hexahydro-azepino[3,2,1-hi]indole-2-carboxylic acid (1H-[1,2,3]triazol-4-ylmethyl)-amide [Scheme 30, Step-124]: To a stirred solution of Acid C178 (100 mg, 0.26 mmol) and central scaffold (117 mg, 0.26 mmol) in DMF (0.8 mL) were added DIPEA (0.28 mL, 1.58 mmol) at 0° C. Finally Bop reagent (232 mg, 0.53 mmol) was added at ice cold condition and the reaction mixture was stirred at room temperature for 16 h. After completion [monitored with LC-MS], crude reaction mixture was submitted for reverse phase prep HPLC purification to afford faster eluting isomer [Isomer 1] of C178 (25 mg, 13%) as white solid. Mass [ESI]: m/z 688.77 [M⁺+1].

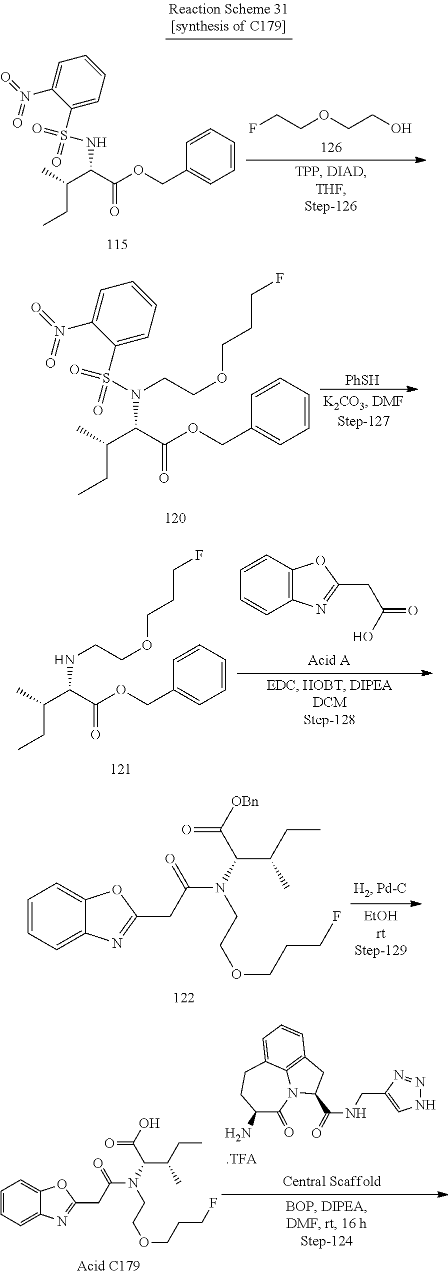

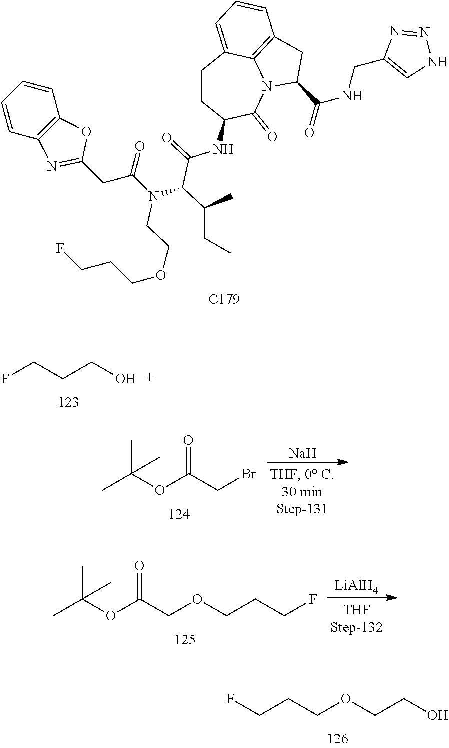

Synthesis of (2S,3S)-2-[[2-(3-Fluoro-propoxy)-ethyl]-(2-nitro-benzenesulfonyl)-amino]-3-methyl-pentanoic acid benzyl ester [Scheme 31, Step-126]: To a stirred solution of Compound 115 (160 mg, 1.31 mmol) and Compound 126 (267 mg, 0.66 mmol) in THF (5.0 mL) was added TPP (340 mg, 1.31 mmol) followed by drop wise addition of DIAD (0.26 mL, 1.31 mmol) at ice cold condition and the reaction mixture was stirred for 4 h at room temperature. After completion (monitored by TLC) reaction mixture was diluted with EtOAc and washed with excess water followed by brine. The organic part was dried [MgSO₄], concentrated and purified by combiflash column chromatography (eluent: 0-5% EtOAc-hexane) to afford compound 120 (140 mg, 21%) as gummy liquid. Mass [ESI]: m/z 510.59 [M⁺+1]

Synthesis of (2S,3S)-2-[2-(3-Fluoro-propoxy)-ethylamino]-3-methyl-pentanoic acid benzyl ester [Scheme 31, Step-127]: To a stirred solution of Compound 120 (140 mg, 0.28 mmol) in DMF (4.0 mL), was added K2CO3 (100 mg, 0.71 mmol) followed by the addition of PhSH (0.03 mL, 0.28 mmol) and the resultant reaction mixture was allowed to stir at room temperature for 1.5 h. After completion of the reaction (confirmed by TLC), the reaction mixture was quenched with ice cold water, extracted with EtOAc, washed with NaHCO₃ solution and brine, dried over Na₂SO₄ and evaporated to get the crude product which was purified by combiflash column chromatography (eluent: 0-20% EtOAc-hexane) to get the desired product compound 121 (65 mg, 72%) as liquid. Mass [ESI]: m/z 325.43 [M⁺+1]

Synthesis of (2S,3S)-2-{(2-Benzooxazol-2-yl-acetyl)-[2-(3-fluoro-propoxy)-ethyl]-amino}-3-methyl-pentanoic acid benzyl ester [Scheme 31, Step-128]: To a stirred solution of Na salt of Acid A (165 mg, 0.83 mmol) and Compound 121 (268 mg, 0.83 mmol) in DCM (4.0 mL) were added DIPEA (0.74 mL, 4.12 mmol) and HOBT (190 mg, 1.24 mmol) at 0° C. followed by the addition of EDC.HCl (206 mg, 1.07 mmol) in one portion at ice cold condition and the reaction mixture was stirred for 16 h at room temperature. After completion [monitored by LC-MS and TLC] reaction mixture was partitioned between DCM and water. Organic layer was separated, dried over sodium sulphate and concentrated under reduced pressure. The resultant crude was purified through combiflash column chromatography (20-40% EtOAc-hexane) to provide compound 122 (210 mg, 50.2%) as white solid. Mass [ESI]: m/z 484.56 [M⁺+1].

Synthesis of (2S,3S)-2-{(2-Benzooxazol-2-yl-acetyl)-[2-(3-fluoro-propoxy)-ethyl]-amino}-3-methyl-pentanoic acid [Scheme 31, Step-129]: To a stirred solution of Compound 122 (210 mg, 0.43 mmol) in EtOH (10 mL), was added Pd—C (100 mg, wet 10%) and resultant suspension was degassed under Argon atmosphere for 10 minutes. Then reaction mixture was stirred for 2 h at room temperature under hydrogen (balloon pressure). After completion (confirmed by TLC), reaction mixture was filtered through celite and washed three times with ethanol. Filtrate was concentrated to afford Acid C179 (170 mg, 99.2%) as off-white solid. Mass [ESI]: m/z 394.45 [M⁺+1].

Synthesis of (3-Fluoro-propoxy)-acetic acid tert-butyl ester [Scheme 31, Step-131]: To a stirred suspension of NaH (60%) (600 mg, 12.8 mmol) in THF (7.0 mL) was added Compound 123 (1.00 gm, 12.8 mmol) at −10° C. and stirred for 5 minutes. Then compound 124 (1.90 mL, 12.8 mmol) was added drop wise at the same temperature and stirred for 10 minutes. After completion (monitored by TLC; 10% EtAOc-Hexane, Rf-0.6) reaction mixture was partitioned between EtOAc and water. Organic part was separated, dried (MgSO₄) and concentrated under reduced pressure. Resultant crude was purified by column chromatography [absorbent SiO₂, eluting solvent 1-2% EtOAc-hexane] to afford compound 125 as light brown liquid. Mass [ESI]: m/z 192.23 [M⁺+1].

Synthesis of 2-(2-Fluoro-ethoxy)-ethanol [Scheme 31, Step-132]: To a suspension of LiAlH₄ (279 mg, 7.34 mmol) in Et₂O (10 mL) was added solution of Compound 125 (470 mg, 7.34 mmol) in Et₂O (10 mL) at 0° C. and allowed to stir at room temperature for 3 h. After completion (monitored with TLC) reaction mixture was slowly quenched with saturated aqueous solution of sodium sulphate under ice cold condition. Filtrate was concentrated under reduced pressure and resultant crude was purified by column chromatography [absorbent-SiO₂, 20-35% EtOAc-hexane] to afford compound 126 (110 mg, 37.3%) as pale yellow liquid. Mass [ESI]: m/z 122.14 [M⁺+1].

Synthesis of (2S,5S)-5-(2S,3S)-2-{(2-Benzooxazol-2-yl-acetyl)-[2-(3-fluoro-propoxy)-ethyl]-amino}-3-methyl-pentanoylamino)-4-oxo-1,2,4,5,6,7-hexahydro-azepino[3,2,1-hi]indole-2-carboxylic acid (1H-[1,2,3]triazol-4-ylmethyl)-amide [Scheme 31, Step-130]: To a stirred solution of Central Scaffold (100 mg, 0.22 mmol) and Acid C190 (88.0 mg, 0.22 mmol) in DMF (2.0 mL) was added DIPEA (0.24 mL, 1.35 mmol), Bop reagent (198 mg, 0.44 mmol) and the resultant reaction mixture was stirred for 16 h at room temperature. After completion [monitored with LC-MS], crude reaction mixture was submitted for reverse phase prep HPLC purification to afford faster eluting isomer [Isomer 1] of C179 (30 mg, 14%) as white solid. Mass [ESI]: m/z 702.79 [M⁺+1].

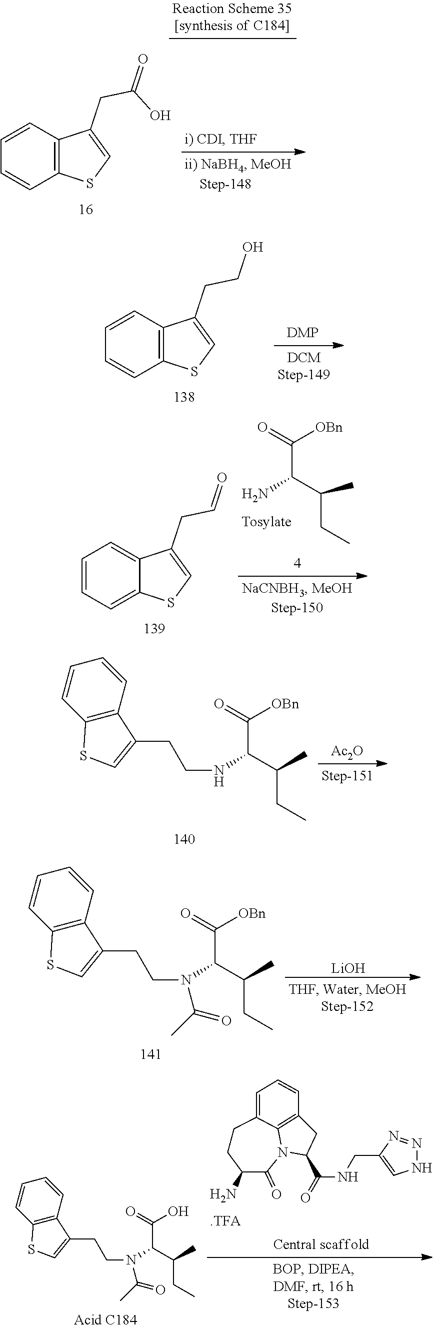

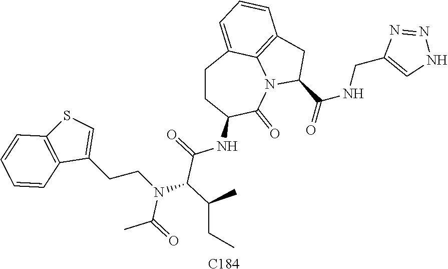 [SMILES: CC[C@H](C)[C@@H](C(=O)N[C@H]1CCc2cccc3c2N(C1=O)[C@H](C(=O)NCc1c[nH]nn1)C3)N(CCc1csc2ccccc12)C(C)=O ; "C184"]

Synthesis of 2-Benzo[b]thiophen-3-yl-ethanol [Scheme 35, Step-148]: To a stirred solution of compound 16 (1.00 gm, 5.20 mmol) in THF (20 mL) was added CDI (928 mg, 5.72 mmol) and the resultant reaction mixture was allowed to stir at 50° C. for 1 h. Then the reaction mixture was brought to room temperature and added to a pre-cooled solution of NaBH₄ (590 mg, 15.6 mmol) in water (40 mL) at 0° C.-5° C. and slowly allowed to reach to room temperature and stirred for 2 h. After completion (monitored by TLC, 30% EtOAc-hexane), the reaction mixture was partitioned between EtOAc and water. The organic part was separated, dried over Na₂SO₄, and evaporated under reduced pressure. Resultant crude was purified by column chromatography (eluent: 10-20% EtOAc-hexane) to afford compound 138 (450 mg, 48%) as colorless sticky liquid. Mass [ESI]: m/z 178.28 [M⁺+1] Synthesis of 2-Benzo[b]thiophen-3-yl-ethanol [Scheme 35, Step-149]: To a stirred solution of compound 138 (480 mg, 2.69 mmol) in DCM (5.0 mL), was added DMP (1.14 gm, 2.69 mmol) at 0° C. and the resultant reaction mixture was allowed to stir at 0-5° C. for 3 h. After completion of the reaction (confirmed by TLC), the reaction mixture was quenched with 10% sodium thiosulfate solution and saturated aqueous sodium bicarbonate solution. Then reaction mixture was extracted with EtOAc, washed with brine, dried (Na₂SO₄) and concentrated. Resultant crude was purified by column chromatography (eluent: 0-15% EtOAc-hexane) to afford desired compound 139 (250 mg, 52%) as colorless liquid. Mass [ESI]: m/z 176.24 [M⁺+1]

Synthesis of (2S,3S)-2-(2-Benzo[b]thiophen-3-yl-ethylamino)-3-methyl-pentanoic acid benzyl ester [Scheme 35, Step-150]: To a stirred solution of compound Amine A (838 mg, 2.13 mmol) in MeOH (2.0 mL), was added TEA (5.90 mL, 4.26 mmol). Then compound 139 (250 mg, 1.42 mmol) was added to the reaction mixture followed the addition of 2 drops of AcOH and the reaction mixture was allowed to stir for 3 h at room temperature. Then NaCNBH₃ (133 mg, 2.13 mmol) was added to the reaction mixture and allowed to stir at room temperature for 16 h. After completion (monitored by LCMS and TLC, 20% EtOAc-hexane), MeOH was evaporated (in low temp) and the reaction mixture was extracted with EtOAc, washed with brine, dried over Na₂SO₄, and evaporated. Resultant crude was purified by column chromatography (eluent: 0-10% EtOAc-hexane, SiO₂) to afford desired compound 140 (180 mg, 33%) as colorless liquid. Mass [ESI]: m/z 381.54 [M⁺+1]

Synthesis of 2S,3S)-2-[Acetyl-(2-benzo[b]thiophen-3-yl-ethyl)-amino]-3-methyl-pentanoic acid benzyl ester [Scheme 35, Step-151]: To a stirred solution of compound 140 (140 mg, 0.37 mmol) in Pyridine (2.0 mL) was added Ac₂O (0.42 mL, 4.40 mmol) and the reaction mixture was allowed to stir for 16 hours at 60° C. After completion of reaction (monitored by TLC and LCMS), reaction mixture was quenched with HCl and extracted with EtOAc. The organic part was washed with brine, dried over Na₂SO₄, and evaporated to provide compound 141 (75.0 mg, 48%) as yellow liquid. Mass [ESI]: m/z 423.58 [M⁺+1]

Synthesis of (2S, 3S)-2-[Acetyl-(2-benzo[b]thiophen-3-yl-ethyl)-amino]-3-methyl-pentanoic acid [Scheme 35, Step-152]: To a stirred solution of compound 141 (75.0 mg, 0.18 mmol) in THF:Water:Methanol (3:1:1, 3 mL), LiOH (22 mg, 0.53 mmol) was added and the reaction mixture was allowed to stir for 4 h at room temperature. After completion [monitored by TLC and LC-MS] reaction mixture was concentrated, resultant crude was partitioned between EtOAc and water. Aqueous part was separated; pH was adjusted to 3-4 using 1 (N) aqueous HCl and extracted with EtOAc. Organic layer was separated, dried over Na₂SO₄, and evaporated to provide compound Acid C184 (50 mg, 84%) as off white solid.

Synthesis of (2S, 5S)-5-{(2S, 3S)-2-[Acetyl-(2-benzo[b]thiophen-3-yl-ethyl)-amino]-3-methyl-pentanoylamino}-4-oxo-1,2,4,5,6,7-hexahydro-azepino[3, 2, 1-hi]indole-2-carboxylic acid (1H-[1, 2, 3]triazol-4-ylmethyl)-amide [Scheme 35, Step-153]: To a stirred solution of Acid C184 (50.0 mg, 0.15 mmol) and central scaffold (66.0 mg, 0.15 mmol) in DMF (2.0 mL), was added DIPEA (0.13 mL, 0.75 mmol), followed by the addition of BOP reagent (133 mg, 0.30 mmol) under argon atmosphere at room temperature and allowed to stir for 16 h. After completion [monitored by LC-MS], crude reaction mixture was submitted for reverse phase preparative HPLC purification to afford faster eluting isomer [Isomer 1] of C184 (8.0 mg, 9%) as white solid. Mass [ESI]: m/z 641.8 [M⁺+1].

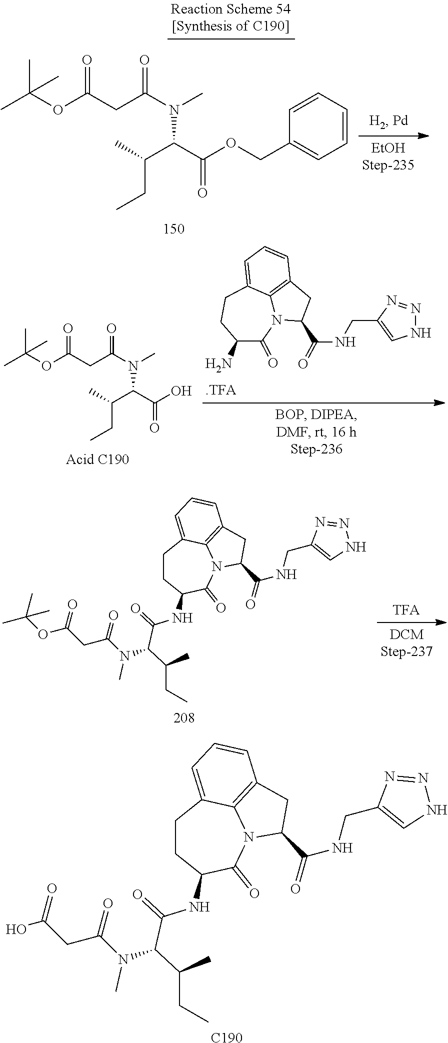

Synthesis of (2S,3S)-2-[(2-tert-Butoxycarbonyl-acetyl)-methyl-amino]-3-methyl-pentanoic acid [Scheme 54, Step-235]: To a solution of compound 150 (300 mg, 0.71 mmol) in EtOH (5.00 mL) was added Pd/C (wet) powder (100 mg) and degassed under argon atmosphere for 10 min. The resultant suspension was allowed to stir under hydrogen balloon pressure for 16 h. After completion (confirmed by TLC), reaction mixture was filtered through celite bed and concentrated under reduced pressure to afford Acid C200 (210 mg, 91.9%) as sticky liquid. Mass [ESI]: m/z 287.36 [M⁺+1] Synthesis of N-Methyl-N-((1S,2S)-2-methyl-1-{(2S,5S)-4-oxo-2-[(1H-[1, 2, 3]triazol-4-ylmethyl)-carbamoyl]-1,2,4,5,6,7-hexahydro-azepino[3,2,1-hi]indol-5-ylcarbamoyl}-butyl)-malonamic acid tert-butyl ester [Scheme 54, Step-236]: To a stirred solution of Acid C200 (130 mg, 0.35 mmol) and Central scaffold (153 mg, 0.35 mmol) in DMF (2.00 mL), was added DIPEA (0.30 mL, 1.74 mmol), Bop reagent (308 mg, 0.61 mmol) and the resultant reaction mixture was allowed to stir at room temperature for 16 h. After completion (confirmed by LC-MS), reaction mixture was extracted by EtOAc, washed with cold-water and brine, dried over solid anhydrous Na₂SO₄. The resultant crude compound 208 (100 mg, 48.2%) was forwarded for the next step. Mass [ESI]: m/z 595.70 [M⁺+1].

Synthesis of N-Methyl-N-((1S, 2S)-2-methyl-1-{(2S,5S)-4-oxo-2-[(1H-[1, 2, 3]triazol-4-ylmethyl)-carbamoyl]-1,2,4,5,6,7-hexahydro-azepino[3,2,1-hi]indol-5-ylcarbamoyl}-butyl)-malonamic acid [Scheme 54, Step-237]: To a stirred solution of compound 208 (100 mg, 0.17 mmol) in DCM (3.00 mL) was added TFA (0.50 mL) at room temperature under argon atmosphere, and stirred for 1.5 h. After completion of the reaction (confirmed by LC-MS), crude reaction mixture was submitted for reverse phase prep HPLC purification to afford faster eluting isomer (Isomer 1, 5.0 mg, 5.50%) of C190 as white solid. Mass [ESI]: m/z 539.60 [M⁺+1].

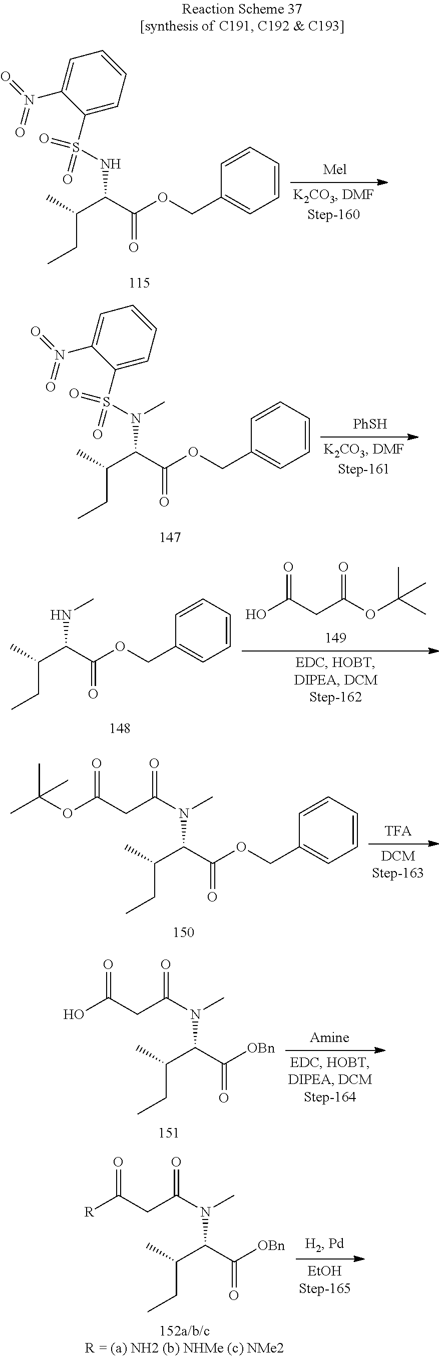

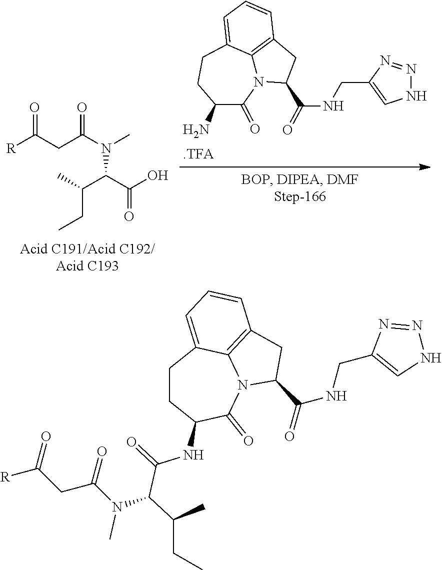

Synthesis of (2S,3S)-3-Methyl-2-[methyl-(2-nitro-benzenesulfonyl)-amino]-pentanoic acid benzyl ester [Scheme 37, Step-160]: To a stirred solution of compound 115 (2.20 gm, 5.41 mmol) in DMF (5.0 mL) was added K2CO3 (1.50 gm, 10.8 mmol) and MeI (1.40 mL, 21.7 mmol) at 0° C. and resultant reaction mixture was allowed to stir at room temperature for 3 h. After completion (monitored by TLC, LCMS) reaction mixture was quenched with ice water, extracted with EtOAc. The organic part was washed with brine, dried over Na₂SO₄, and evaporated to provide crude compound 147 (2.3 gm, 99%) as yellow sticky liquid. Mass [ESI]: m/z 420.49 [M⁺+1]

Synthesis of (2S,3S)-3-Methyl-2-methylamino-pentanoic acid benzyl ester [Scheme 37, Step-161]: To a stirred solution of compound 147 (2.20 gm, 5.23 mmol) in DMF (5.0 mL), was added K2CO3 (1.88 gm, 13.6 mmol) followed by the addition of PhSH (0.53 mL, 5.23 mmol) and the resultant reaction mixture was allowed to stir at room temperature for 3 h. After completion (confirmed by TLC), reaction mixture was quenched with ice cold water, extracted with EtOAc. Organic layer was further washed with NaHCO₃ and brine, dried over Na₂SO₄ and evaporated under reduced pressure. Crude residue was purified by combiflash (eluent: 0-20% EtOAc-hexane) to provide pure compound 148 (900 mg, 73.2%) as yellow liquid. Mass [ESI]: m/z 235.33 [M⁺+1]

Synthesis of (2S,3S)-2-[(2-tert-Butoxycarbonyl-acetyl)-methyl-amino]-3-methyl-pentanoic acid benzyl ester[Scheme 37, Step-162]: To a stirred solution of compound 148 (650 mg, 2.76 mmol) in DCM (3.0 mL) was added compound 149 (442 mg, 2.76 mmol), DIPEA (2.40 mL, 13.8 mmol), HOBT (571 mg, 4.23 mmol), EDC.HCl (704 mg, 3.67 mmol) at room temperature under inert atmosphere and allowed stir for 16 h. After completion (confirmed by LC-MS), reaction mixture was partitioned between DCM and water. Organic part was further washed with brine, dried over Na₂SO₄, and evaporated under reduced pressure. Resultant crude was purified by combiflash (eluent: 0-20% EtOAc-hexane) to afford compound 150 (780 mg, 74.1%) as colorless sticky liquid. Mass [ESI]: m/z 377.48 [M⁺+1]

Synthesis of (2S,3S)-2-[(2-Carboxy-acetyl)-methyl-amino]-3-methyl-pentanoic acid benzyl ester [Scheme 37, Step-163]: To a stirred solution of compound 150 (780 mg, 2.06 mmol) in DCM (5.0 mL) was added 50 TFA: DCM (5.0 mL) and the reaction mixture was stirred for 3 h at room temperature. After completion (monitored by LC-MS) reaction mixture was concentrated under reduced pressure to provide compound 151 (900 mg, 90.2%) as brown sticky liquid. Mass [ESI]: m/z 321.38 [M++1]

### Synthesis of C191

Synthesis of (2S,3S)-2-[(2-Carbamoyl-acetyl)-methyl-amino]-3-methyl-pentanoic acid benzyl ester [Scheme 37, Step-164]: To a stirred solution of compound 151 (400 mg, 0.92 mmol) in DMF (4.0 mL) was added DIPEA (0.8 mL, 4.6 mmol), NH₄Cl (492 mg, 9.20 mmol), TBTU (443 mg, 1.38 mmol) and the resultant reaction mixture was allowed to stir at room temperature for 16 h. After completion (confirmed by LC-MS), reaction mixture was partitioned between EtOAc and water. Organic layer was further washed with brine, dried over Na₂SO₄ and evaporated under reduced pressure. The resultant crude was purified by combiflash column chromatography (eluent: 30-60% EtOAc-hexane) to provide the compound 152a (160 mg, 54.2%) as white solid. Mass [ESI]: m/z 320.39 [M⁺+1]

Synthesis of (2S,3S)-2-[(2-Carbamoyl-acetyl)-methyl-amino]-3-methyl-pentanoic acid [Scheme 37, Step-165]: To a solution of compound 152a (160 mg, 0.50 mmol) in EtOH (3.0 mL) was added Pd/C (wet) powder (50 mg) and degassed under argon atmosphere for 10 min. The resultant suspension was allowed to stir under hydrogen balloon pressure for 16 h. After completion (confirmed by TLC), reaction mixture was filtered through celite bed and concentrated under reduced pressure to afford Acid C191 (100 mg, 87.2%) as sticky liquid. Mass [ESI]: m/z 230.27 [M⁺+1]

Synthesis of N-Methyl-N-((1S,2S)-2-methyl-1-{(2S,5S)-4-oxo-2-[(1H-[1, 2, 3]triazol-4-ylmethyl)-carbamoyl]-1,2,4,5,6,7-hexahydro-azepino[3,2,1-hi]indol-5-ylcarbamoyl}-butyl)-malonamide [Scheme 37, Step-166]: To a stirred solution of Acid C191 (80.0 mg, 0.35 mmol) and Central scaffold (153 mg, 0.35 mmol) in DMF (2.0 mL), was added DIPEA (0.3 mL, 1.7 mmol), Bop reagent (307 mg, 0.70 mmol) and the resultant reaction mixture was allowed to stir at room temperature for 16 h. After completion [monitored with LC-MS], crude reaction mixture was submitted for reverse phase prep HPLC purification to afford faster eluting isomer [Isomer 1] compound C191 (18 mg, 9.2%) as white solid. Mass [ESI]: m/z 538.61 [M⁺+1]

### Synthesis of C192

Synthesis of (2S,3S)-3-Methyl-2-[methyl-(2-methylcarbamoyl-acetyl)-amino]-pentanoic acid benzyl ester [Scheme 37, Step-164]: To a stirred solution of compound 151 (200 mg, 0.46 mmol) in DCM (5.0 mL) was added methyl amine (31.0 mg, 0.46 mmol), DIPEA (0.4 mL, 2.3 mmol), HOBT (80.0 mg, 0.60 mmol), EDC.HCl (131 mg, 0.69 mmol) and allowed to stir at room temperature for 16 h. After completion (monitored with LC-MS), reaction mixture was extracted with DCM. Organic layer was further washed with brine, dried (MgSO₄) and concentrated. The crude residue was purified by combiflash column chromatography (eluent: 20-40% EtOAc-hexane) to afford compound 152b (110 mg, 71%) as white solid. Mass [ESI]: m/z 334.42 [M⁺+1]

Synthesis of (2S,3S)-3-Methyl-2-[methyl-(2-methylcarbamoyl-acetyl)-amino]-pentanoic acid [Scheme 37, Step-165]: To a solution of compound 152b (160 mg, 0.55 mmol) in EtOH (5.0 mL) was added Pd/C (wet) powder (70 mg) and degassed under argon atmosphere for 15 minutes. Then reaction mixture was stirred under hydrogen pressure (balloon) for 16 h. After completion (confirmed by TLC), reaction mixture was filtered through celite bed. Filtrate was concentrated under reduced pressure to afford Acid C192 (100 mg, 75.2%) as sticky liquid. Mass [ESI]: m/z 244.29 [M⁺+1]

Synthesis of N,N′-Dimethyl-N-((1S,2S)-2-methyl-1-{(2S,5S)-4-oxo-2-[(1H-[1,2,3]triazol-4-ylmethyl)-carbamoyl]1,2,4,5,6,7-hexahydro-azepino[3,2,1-hi] indol-5-ylcarbamoyl}-butyl)-malonamide [Scheme 37, Step-166]: To a stirred solution of Acid C192 (83.2 mg, 0.34 mmol) in DMF (2.0 mL) was added central scaffold (150 mg, 0.34 mmol), DIPEA (0.28 mL, 1.70 mmol), BOP reagent (301 mg, 0.68 mmol) and allowed to stir at room temperature for 16 h. After completion [monitored with LC-MS], crude reaction mixture was submitted for reverse phase prep HPLC purification to afford faster eluting isomer [Isomer 1] (5.3 mg) of pure compound C192 as white solid. Mass [ESI]: m/z 552.64 [M⁺+1]

### Synthesis of C193

Synthesis of (2S,3S)-2-[(2-Dimethylcarbamoyl-acetyl)-methyl-amino]-3-methyl-pentanoic acid benzyl ester [Scheme 37, Step-164]: To a stirred solution of compound 151 (200 mg, 0.46 mmol) in DCM (3.0 mL) was added Dimethyl amine (37.5 mg, 0.46 mmol), DIPEA (0.40 mL, 2.30 mmol), HOBT (82.6 mg, 0.61 mmol) and EDC.HCl (135 mg, 0.70 mmol) at room temperature under inert atmosphere and the reaction mixture was stirred for 16 h. After completion (confirmed by LC-MS), reaction mixture was partitioned between DCM and water. Organic layer was separated, further washed with brine, dried (Na₂SO₄) and concentrated under reduced pressure. Resultant crude was purified by combiflash column chromatography (eluent: 50-70% EtOAc-hexane) to afford compound 152C (108 mg, 67.4%) as white solid. Mass [ESI]: m/z 348.45 [M⁺+1]

Synthesis of (2S,3S)-2-[(2-Dimethylcarbamoyl-acetyl)-methyl-amino]-3-methyl-pentanoic acid [Scheme 37, Step-165]: To a solution of compound 152C (150 mg, 0.43 mmol) in ethanol (5.0 mL) was added 10% by weight Pd/C (wet) powder (60 mg) and degassed under argon atmosphere for 15 minutes. Then the reaction mixture was stirred under hydrogen balloon pressure for 16 h. After completion (confirmed by TLC) reaction mixture was filtered through celite bed, filtrate was concentrated under reduced pressure to afford Acid C193 (90 mg, 81%) as sticky liquid. Mass [ESI]: m/z 258.32 [M⁺+1]

Synthesis of N,N,N-Trimethyl-N′-((1S,2S)-2-methyl-1-{(2S,5S)-4-oxo-2-[(1H-[1,2,3]triazol-4-ylmethyl)-carbamoyl]-1,2,4,5,6,7-hexahydro-azepino[3,2,1-hi]indol-5-ylcarbamoyl}-butyl)-malonamide [Scheme 37, Step-166]: To a stirred solution of Acid C193 (88.0 mg, 0.34 mmol) and central scaffold (150 mg, 0.34 mmol) in DMF (2.0 mL), was added DIPEA (0.3 mL, 1.7 mmol), Bop reagent (301 mg, 0.68 mmol) and resultant reaction mixture was allowed to stir at room temperature for 16 h. After completion [monitored with LC-MS], crude reaction mixture was submitted for reverse phase prep HPLC purification to afford faster eluting isomer [Isomer 1] (14 mg) of C193 as white solid. Mass [ESI]: m/z 566.67 [M⁺+1]

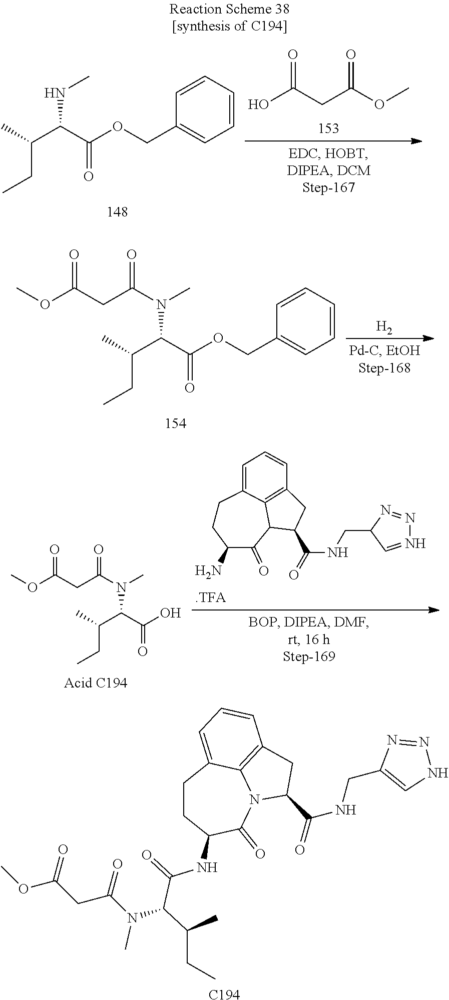

Synthesis of (2S,3S)-2-[(2-Methoxycarbonyl-acetyl)-methyl-amino]-3-methyl-pentanoic acid benzyl ester [Scheme 38, Step-167]: To a stirred solution of compound 148 (340 mg, 1.45 mmol) in DCM (3.0 mL) was added compound 153 (226 mg, 1.45 mmol), DIPEA (1.30 mL, 7.23 mmol), HOBT (293 mg, 2.17 mmol), EDC.HCl (360 mg, 1.88 mmol) and allowed to stir at room temperature for 16 h. After completion (confirmed by TLC), reaction mixture was partitioned between DCM and water. Organic layer was separated, further washed with brine, dried over Na₂SO₄ and concentrated under reduced pressure. Resultant crude was purified by combiflash column chromatography (eluent: 0-20% EtOAc-hexane) to afford compound 154 (390 mg, 80%) as colorless sticky liquid. Mass [ESI]: m/z 335.40 [M⁺+1]

Synthesis of (2S,3S)-2-[(2-Methoxycarbonyl-acetyl)-methyl-amino]-3-methyl-pentanoic acid [Scheme 38, Step-168]: To a solution of compound 154 (390 mg, 1.16 mmol) in EtOH (4.0 mL) was added 10% by weight Pd/C (wet) (170 mg) and degassed under argon atmosphere for 15 minutes. Then reaction mixture was stirred under hydrogen balloon pressure for 16 h. After completion (confirmed by TLC), reaction mixture was filtered through celite bed. The filtrate was concentrated under reduced pressure to provide Acid C194 (280 mg, 99.2%) as sticky liquid. Mass [ESI]: m/z 245.28 [M⁺+1]

Synthesis of N-Methyl-N-((1S, 2S)-2-methyl-1-{(2S, 5S)-4-oxo-2-[(1H-[1, 2, 3]triazol-4-ylmethyl)-carbamoyl]-1, 2, 4, 5, 6, 7-hexahydro-azepino[3,2,1-hi]indol-5-ylcarbamoyl}-butyl)-malonamic acid methyl ester [Scheme 38, Step-169]: To a stirred solution of Acid C194 (80.0 mg, 0.33 mmol) and central scaffold (143 mg, 0.33 mmol) in DMF (2.0 mL) was added DIPEA (0.28 mL, 1.63 mmol) drop wise, followed by the addition of BOP reagent (289 mg, 0.65 mmol) under argon atmosphere at room temperature. The reaction was continued at the same temperature for 16 h. After completion [monitored with LC-MS], crude reaction mixture was submitted for reverse phase prep HPLC purification to afford faster eluting isomer [Isomer 1] (15.0 mg) of C194 as white solid. Mass [ESI]: m/z 553.62 [M⁺+1].

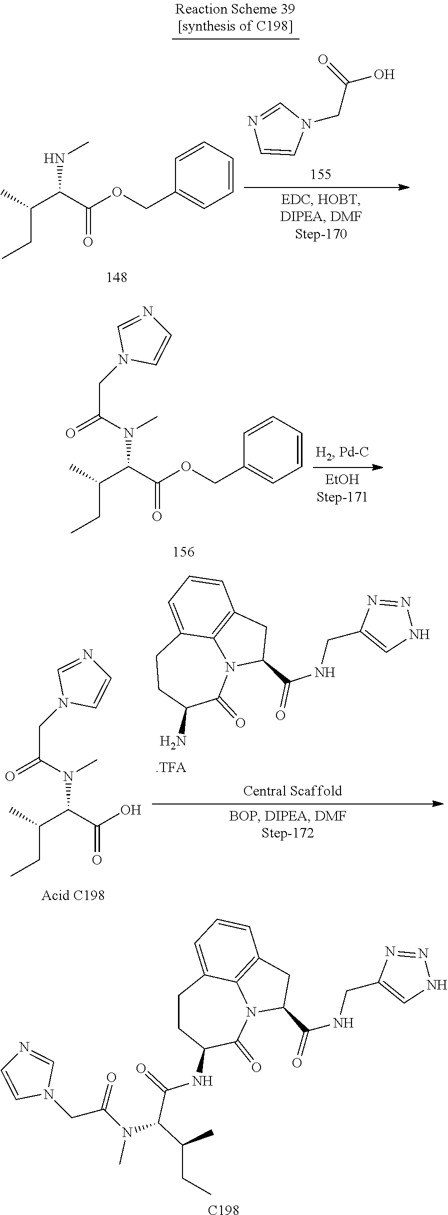

Synthesis of (2S,3S)-3-Methyl-2-methylamino-pentanoic acid benzyl ester [Scheme 39, Step-170]: To the stirred solution of compound 148 (200 mg, 0.85 mmol) in DMF (4.0 mL) was added compound 155 (107 mg, 0.85 mmol), DIPEA (0.55 mL, 4.26 mmol), HOBT (172 mg, 1.28 mmol), EDC.HCl (211 mg, 1.11 mmol) and resultant reaction mixture was allowed to stir at room temperature for 16 h. After completion (confirmed by LCMS) reaction mixture was partitioned between EtOAc and excess of water. Organic layer was separated, dried (MgSO₄) and concentrated under reduced pressure. Resultant crude was purified by column chromatography (eluent: 0-2% Methanol-DCM, SiO₂) to afford pure compound 156 (180 mg, 61.2%) as sticky liquid. Mass [ESI]: m/z 343.43 [M⁺+1]

Synthesis of (2S,3S)-2-[(2-Imidazol-1-yl-acetyl)-methyl-amino]-3-methyl-pentanoic acid [Scheme 39, Step-171]: To the solution of compound 156 (180 mg, 0.53 mmol) in EtOH (10 mL) was added 10% by weight Pd/C (wet) (100 mg) and degassed under argon atmosphere for 15 minutes. Then resultant reaction mixture was stirred for 16 h at room temperature under hydrogen balloon pressure. After completion (confirmed by TLC), reaction mixture was filtered through celite bed. The filtrate was concentrated under reduced pressure and the resultant crude was triturated with n-pentane to afford Acid C198 (100 mg, 75.2%) as white solid. Mass [ESI]: m/z 253.30 [M⁺+1]

Synthesis of (2S,5S)-5-{(2S,3S)-2-[(2-Imidazol-1-yl-acetyl)-methyl-amino]-3-methyl-pentanoylamino}-4-oxo-1,2,4,5,6,7-hexahydro-azepino [3,2,1-hi]indole-2-carboxylic acid (1H-[1,2,3]triazol-4-ylmethyl)-amide [Scheme 39, Step-172]: To a stirred solution of Acid C198 (102 mg, 0.40 mmol) and Central Scaffold (150 mg, 0.34 mmol) in DMF (2.0 mL) was added DIPEA (0.30 mL, 1.68 mmol) and Bop reagent (297 mg, 0.67 mmol). Then the resultant reaction mixture was allowed to stir at room temperature for 16 h. After completion [monitored with LC-MS], crude reaction mixture was submitted for reverse phase prep HPLC purification to afford faster eluting isomer [Isomer 1] (72.0 mg, 38.1%) of C198 as white solid. Mass [ESI]: m/z 561.65 [M⁺+1]

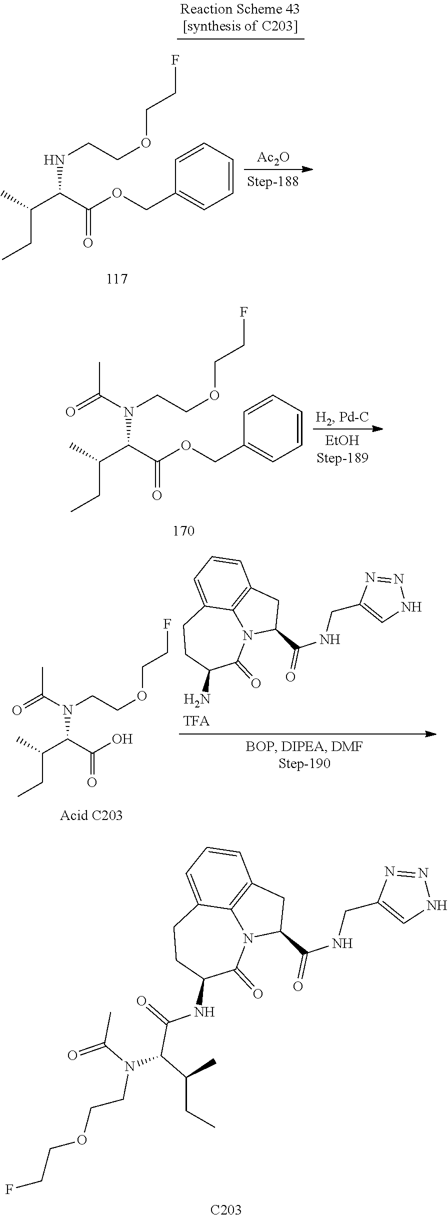

Synthesis of (2S,3S)-2-{Acetyl-[2-(2-fluoro-ethoxy)-ethyl]-amino}-3-methyl-pentanoic acid benzyl ester [Scheme 43, Step-188]: To a stirred solution of compound 117 (380 mg, 1.22 mmol) in Pyridine (2.5 mL) was added Ac₂O (1.20 mL, 12.2 mmol) and the reaction mixture was allowed to stir for 16 h at 60° C. After completion (monitored by TLC and LCMS), reaction mixture was quenched with aqueous HCl, extracted with EtOAc. Organic part was washed with brine, dried over Na₂SO₄, and concentrated under reduced pressure. Resultant crude was purified by column chromatography (eluent: 0-20% EtOAc-hexane, absorbent SiO₂) to afford compound 170 (750 mg, 79%) as sticky liquid. Mass [ESI]: m/z 353.43 [M⁺+1].

Synthesis of (2S,3S)-2-{Acetyl-[2-(2-fluoro-ethoxy)-ethyl]-amino}-3-methyl-pentanoic acid [Scheme 43, Step-189]: To a stirred solution of Compound 170 (350 mg, 0.99 mmol) in EtOH (20 mL) was added 10% Pd/C (wet) powder (200 mg) and degassed under argon atmosphere for 10 minutes. Then reaction mixture was stirred at room temperature for overnight under hydrogen balloon pressure. After completion (confirmed by TLC), reaction mixture was filtered through celite, filtrate was concentrated to provide crude compound Acid C203 (260 mg, 99%) as gummy liquid. Mass [ESI]: m/z 263.31 [M⁺+1]

Synthesis of (2S, 5S)-5-((2S,3S)-2-{Acetyl-[2-(2-fluoro-ethoxy)-ethyl]-amino}-3-methyl-pentanoylamino)-4-oxo-1, 2, 4, 5, 6, 7-hexahydro-azepino[3, 2, 1-hi] indole-2-carboxylic acid (1H[1, 2, 3]triazol-4-ylmethyl)-amide [Scheme 43, Step-190]: To a stirred solution of TFA salt of Central Scaffold (200 mg, 0.45 mmol) and Acid C203 (138 mg, 0.45 mmol) in DMF (2.0 mL) was added DIPEA (0.40 mL, 2.26 mmol) followed by addition of BOP reagent (400 mg, 0.91 mmol) under argon atmosphere at room temperature. The reaction mixture was continued at ambient temperature for 16 h. After completion [monitored with LC-MS], crude reaction mixture was submitted for reverse phase prep HPLC purification to afford faster eluting isomer [Isomer 1] (37.0 mg, 14%) of C203 as white solid. Mass [ESI]: m/z 571.66 [M⁺+1].

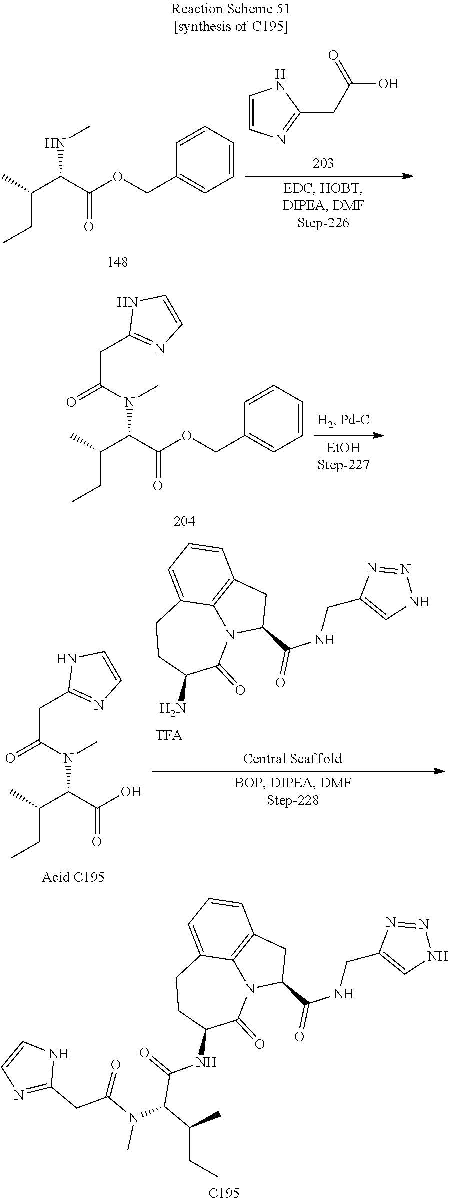

Synthesis of (2S,3S)-2-[(2-1H-Imidazol-2-yl-acetyl)-methyl-amino]-3-methyl-pentanoic acid benzyl ester [Scheme 51, Step-226]: To the stirred solution of compound 148 (300 mg, 2.38 mmol) in DMF (4.0 mL) was added compound 203 (560 mg, 2.38 mmol), DIPEA (2.13 mL, 11.90 mmol), HOBT (482 mg, 3.57 mmol), EDC.HCl (591 mg, 3.10 mmol) and resultant reaction mixture was allowed to stir at room temperature for 16 h. After completion (confirmed by LCMS) reaction mixture was partitioned between EtOAc and excess of water. Organic layer was separated, dried (MgSO₄) and concentrated under reduced pressure. Resultant crude was purified by column chromatography (eluent: 0-2% Methanol-DCM, SiO₂) to afford pure compound 204 (200 mg, 24.5%) as sticky liquid. Mass [ESI]: m/z 343.43 [M⁺+1].

Synthesis of (2S,3S)-2-[(2-1H-Imidazol-2-yl-acetyl)-methyl-amino]-3-methyl-pentanoic acid [Scheme 51, Step-227]: To the solution of compound 204 (200 mg, 0.58 mmol) in EtOH (10.0 mL) was added 10% by weight Pd/C (wet) (120 mg) and degassed under argon atmosphere for 15 minutes. Then resultant reaction mixture was stirred for 16 h at room temperature under hydrogen balloon pressure. After completion (confirmed by TLC), reaction mixture was filtered through celite bed. The filtrate was concentrated under reduced pressure and the resultant crude was triturated with n-pentane to afford Acid C195 (130 mg, 88.0%) as white solid. Mass [ESI]: m/z 253.30 [M⁺+1].

Synthesis of (2S, 5S)-5-{(2S, 3S)-2-[(2-1H-Imidazol-2-yl-acetyl)-methyl-amino]-3-methyl-pentanoylamino}-4-oxo-1,2,4,5,6,7-hexahydro-azepino [3,2,1-hi]indole-2-carboxylic acid (1H-[1,2,3]triazol-4-ylmethyl)-amide [Scheme 51, Step-228]: To a stirred solution of Acid (102 mg, 0.40 mmol) and Central Scaffold (150 mg, 0.34 mmol) in DMF (2.0 mL) was added DIPEA (0.30 mL, 1.68 mmol) and Bop reagent (297 mg, 0.67 mmol). Then the resultant reaction mixture was allowed to stir at room temperature for 16 h. After completion [monitored with LC-MS], crude reaction mixture was submitted for reverse phase prep HPLC purification to afford faster eluting isomer [Isomer 1] (5.0 mg, 2.60%) of C195 as white solid. Mass [ESI]: m/z 561.65 [M⁺+1].

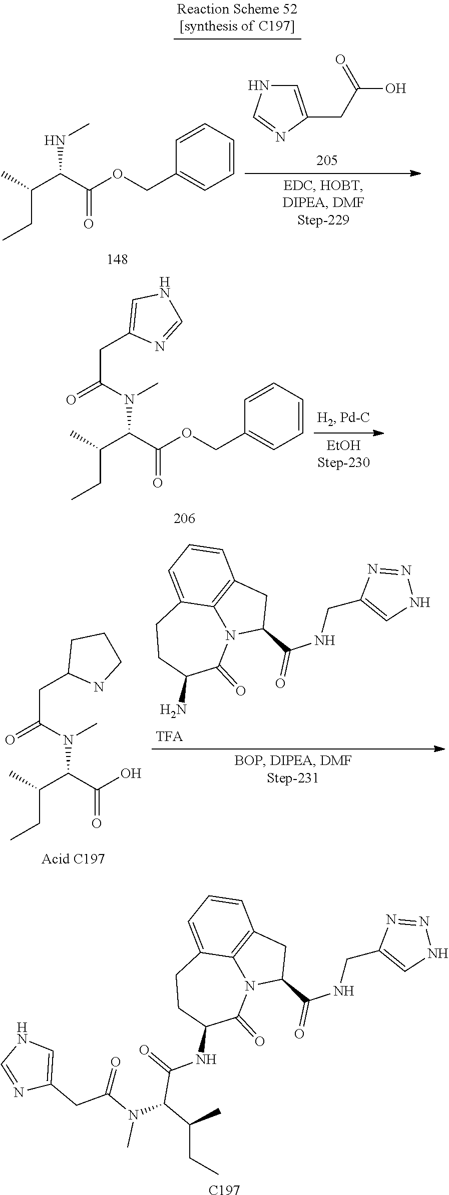

Synthesis of (2S,3S)-2-[(2-1H-Imidazol-2-yl-acetyl)-methyl-amino]-3-methyl-pentanoic acid benzyl ester [Scheme 52, Step-229]: To the stirred solution of compound 148 (350 mg, 1.49 mmol) in DMF (4.0 mL) was added compound 205 (242 mg, 1.49 mmol), DIPEA (1.34 mL, 7.45 mmol), HOBT (302 mg, 2.23 mmol), EDC.HCl (370 mg, 1.94 mmol) and resultant reaction mixture was allowed to stir at room temperature for 16 h. After completion (confirmed by LCMS) reaction mixture was partitioned between EtOAc and excess of water. Organic layer was separated, dried (MgSO₄) and concentrated under reduced pressure. Resultant crude was purified by column chromatography (eluent: 0-2% Methanol-DCM, Sift) to afford pure compound 206 (140 mg, 27.4%) as sticky liquid. Mass [ESI]: m/z 343.43 [M⁺+1].

Synthesis of (2S,3S)-2-[(2-1H-Imidazol-2-yl-acetyl)-methyl-amino]-3-methyl-pentanoic acid [Scheme 52, Step-230]: To the solution of compound 206 (180 mg, 0.53 mmol) in EtOH (10.0 mL) was added 10% by weight Pd/C (wet) (110 mg) and degassed under argon atmosphere for 15 minutes. Then resultant reaction mixture was stirred for 16 h at room temperature under hydrogen balloon pressure. After completion (confirmed by TLC), reaction mixture was filtered through celite bed. The filtrate was concentrated under reduced pressure and the resultant crude was triturated with n-pentane to afford C197 (120 mg, 90.3%) as white solid. Mass [ESI]: m/z 253.30 [M⁺+1].

Synthesis of (2S, 5S)-5-{(2S, 3S)-2-[(2-1H-Imidazol-4-yl-acetyl)-methyl-amino]-3-methyl-pentanoylamino}-4-oxo-1,2,4,5,6,7-hexahydro-azepino[3, 2, 1-hi]indole-2-carboxylic acid (1H-[1,2,3]triazol-4-ylmethyl)-amide [Scheme 52, Step-231]: To a stirred solution of Acid C197 (88.49 mg, 0.35 mmol) and Central Scaffold (130 mg, 0.29 mmol) in DMF (2.0 mL) was added DIPEA (0.26 mL, 1.46 mmol) and Bop reagent (258 mg, 0.58 mmol). Then the resultant reaction mixture was allowed to stir at room temperature for 16 h. After completion [monitored with LC-MS], crude reaction mixture was submitted for reverse phase prep HPLC purification to afford faster eluting isomer [Isomer 1] (8.80 mg, 4.20%) of C197 as white solid. Mass [ESI]: m/z 561.65 [M⁺+1]

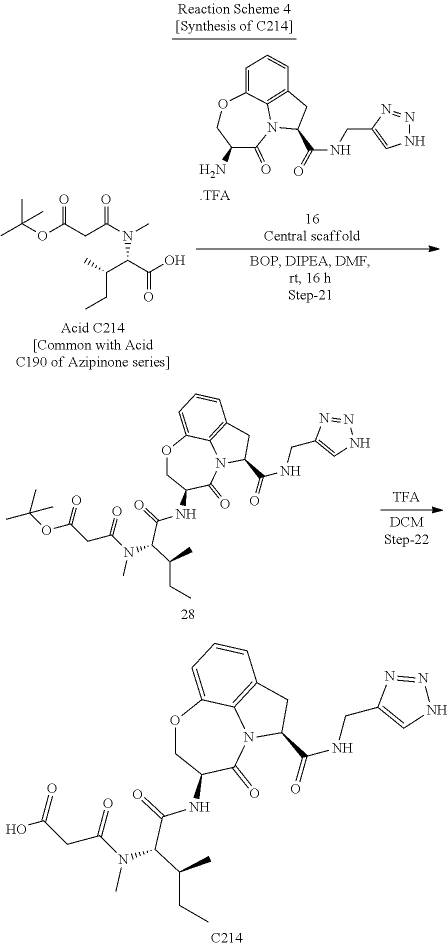

Synthesis of N-Methyl-N-((1S, 2S)-2-methyl-1-{(1S, 8S)-9-oxo-1-[(1H-[1, 2, 3]triazol-4-ylmethyl)-carbamoyl]-1, 2, 8, 9-tetrahydro-7H-6-oxa-9a-aza-benzo [cd]azulen-8-ylcarbamoyl}-butyl)-malonamic acid tert-butyl ester [Scheme 4, Step-21]: To a stirred solution of Acid C 225 (65.0 mg, 0.23 mmol) and Central scaffold (compound 16, 100 mg, 0.23 mmol) in DMF (2.00 mL), was added DIPEA (0.20 mL, 1.13 mmol), BOP reagent (200 mg, 0.45 mmol) and the resultant reaction mixture was allowed to stir at room temperature for 16 h. After completion (confirmed by LC-MS), reaction mixture was extracted by EtOAc, washed with cold-water and brine, dried over solid anhydrous Na₂SO₄ and concentrated under reduced pressure. The resultant crude compound 28 (150 mg) was forwarded for the next step without further purification. Mass [ESI]: m/z 597.68 [M⁺+1].

Synthesis of N-Methyl-N-((1S, 2S)-2-methyl-1-{(1S, 8S)-9-oxo-1-[(1H-[1, 2, 3]triazol-4-ylmethyl)-carbamoyl]-1, 2, 8, 9-tetra hydro-7H-6-oxa-9a-aza-benzo[cd]azulen-8-ylcarbamoyl}-butyl)-malonamic acid [Scheme 4, Step-22]: To a stirred solution of compound 28 (150 mg, 0.25 mmol) in DCM (0.50 mL) was added TFA (1.50 mL) at room temperature under argon atmosphere and stirred for 5 h. After completion of the reaction (confirmed by LC-MS), crude reaction mixture was submitted for reverse phase prep HPLC purification to afford faster eluting isomer (Isomer 1) (8.00 mg, 5.90%) of C214 as white solid. Mass [ESI]: m/z 541.57 [M⁺+1].

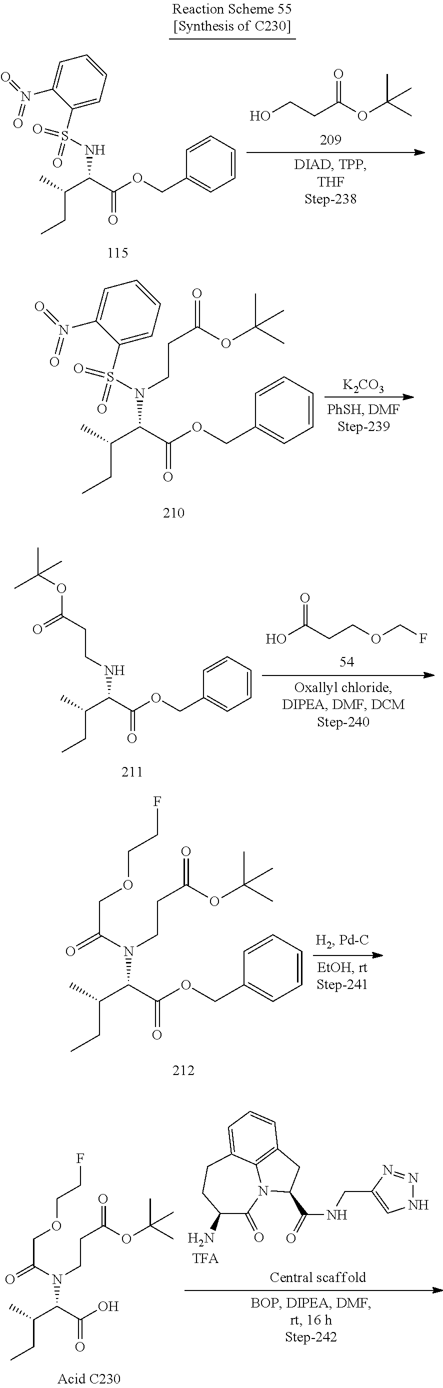

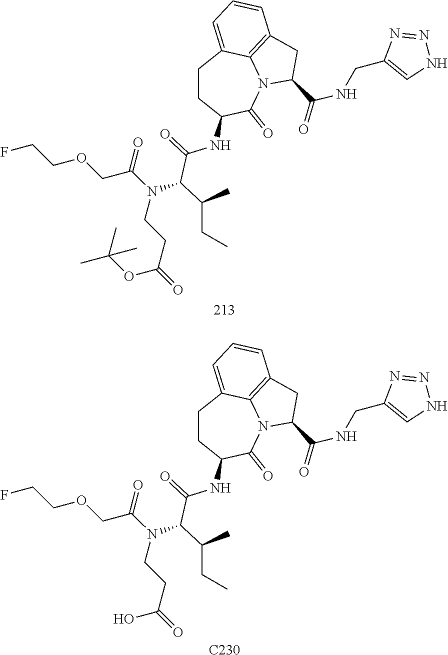 [SMILES: CC[C@H](C)[C@@H](C(=O)N[C@H]1CCc2cccc3c2N(C1=O)[C@H](C(=O)NCc1c[nH]nn1)C3)N(CCC(=O)OC(C)(C)C)C(=O)COCCF ; CC[C@H](C)[C@@H](C(=O)N[C@H]1CCc2cccc3c2N(C1=O)[C@H](C(=O)NCc1c[nH]nn1)C3)N(CCC(=O)O)C(=O)COCCF ; "C230"]

Synthesis of (2S,3S)-2-[(2-tert-Butoxycarbonyl-ethyl)-(2-nitro-benzenesulfonyl)-amino]-3-methyl-pentanoic acid benzyl ester [Scheme 55, Step-238]: To a stirred solution of Compound 115 (1.00 gm, 2.46 mmol) and Compound 209 (0.72 gm, 4.92 mmol) in THF (5.00 mL) was added TPP (1.29 gm, 4.92 mmol) followed by drop wise addition of DIAD (0.97 mL, 4.92 mmol) at ice cold condition and the reaction mixture was stirred for 4 h at room temperature. After completion (monitored by TLC) reaction mixture was diluted with EtOAc and washed with excess water followed by brine: The organic part was dried [MgSO₄], concentrated and purified by combiflash column chromatography (eluent: 0-5% EtOAc-hexane) to afford compound 210 (250 mg, 19%) as gummy liquid. Mass [ESI]: m/z 534.63 [M⁺+1]

Synthesis of (2S,3S)-2-(2-tert-Butoxycarbonyl-ethylamino)-3-methyl-pentanoic acid benzyl ester [Scheme 55, Step-239]: To a stirred solution of Compound 210 (500 mg, 0.94 mmol) in DMF (4.0 mL), was added K2CO3 (336 mg, 2.43 mmol) followed by the addition of PhSH (0.12 mL, 1.12 mmol) and the resultant reaction mixture was allowed to stir at room temperature for 3 h. After completion of the reaction (confirmed by TLC), the reaction mixture was quenched with ice cold water, extracted with EtOAc, washed with NaHCO₃ solution and brine, dried over Na₂SO₄ and evaporated under reduced pressure. Resultant crude was purified by combiflash column chromatography (eluent: 0-10% EtOAc-hexane) to provide desired compound 211 (170 mg, 52.1%) as yellow liquid. Mass [ESI]: m/z 349.47 [M⁺+1]

Synthesis of (2S,3S)-2-{(2-tert-Butoxycarbonyl-ethyl)-[2-(2-fluoro-ethoxy)-acetyl]-amino}-3-methyl-pentanoic acid benzyl ester [Scheme 55, Step-240]: To a stirred solution of compound 211 (100 mg, 0.82 mmol) in DCM (5.00 mL) was added oxalyl chloride (0.14 mL, 1.64 mmol) followed by addition of catalytic amount of DMF and the resultant reaction mixture was allowed to stir at room temperature for 30 minutes. Then reaction mixture was concentrated under reduced pressure and resultant crude acid chloride was diluted with DCM (2.00 mL) followed by addition of DIPEA (0.71 mL, 4.09 mmol), compound 54 (286 mg, 0.82 mmol) and allowed to stir at room temperature for 16 h. After completion (confirmed by TLC), the resultant reaction mixture was partitioned between EtOAc and water. Organic layer was separated, dried (Na₂SO₄) and concentrated under reduced pressure. Crude residue thus obtained was purified by column chromatography (eluent: 0-20% EtOAc-hexane) to afford compound 212 [75.0 mg] as light brown sticky liquid. Mass [ESI]: m/z 453.56 [M⁺+1].

Synthesis of (2S,3S)-2-{(2-tert-Butoxycarbonyl-ethyl)-[2-(2-fluoro-ethoxy)-acetyl]-amino}-3-methyl-pentanoic acid [Scheme 55, Step-241]: To a stirred solution of Compound 212 [100 mg, 0.22 mmol] in EtOH [3.00 mL] was added Pd—C [40.0 mg, 10% wet] and degassed under argon atmosphere for 10 min. The resultant suspension was subjected to ordinary hydrogen pressure (balloon) and allowed to stir at room temperature for 16 h. After completion [confirmed by TLC], reaction mixture was filtered through celite bed and concentrated under reduced pressure to provide Acid C230 (60.0 mg) as colorless liquid. Mass [ESI]: m/z 363.43 [M⁺+1].

Synthesis of 3-[[2-(2-Fluoro-ethoxy)-acetyl]-((1S,2S)-2-methyl-1-{(2S,5S)-4-oxo-2-[(1H-[1,2,3]triazol-4-ylmethyl)-carbamoyl]-1,2,4,5,6,7-hexahydro-azepino[3,2,1-hi]indol-5-ylcarbamoyl}-butyl)-amino]-propionic acid tert-butyl ester [Scheme 55, Step-242]: To a stirred solution of Central Scaffold (72.7 mg, 0.17 mmol) and Acid C230 (60.0 mg, 0.17 mmol) in DMF (2.00 mL) was added DIPEA (0.14 mL, 0.83 mmol), BOP reagent (146 mg, 0.33 mmol) and the resultant reaction mixture was allowed to stir at room temperature for 16 h. After completion (confirmed by LC-MS), reaction mixture was extracted by EtOAc, washed with cold-water and brine, dried over solid anhydrous Na₂SO₄ and concentrated. The resultant crude compound 213 (150 mg) was forwarded for the next step without further purification. Mass [ESI]: m/z 671.78 [M⁺+1].

Synthesis of 3-[[2-(2-Fluoro-ethoxy)-acetyl]-((1S,2S)-2-methyl-1-{(2S,5S)-4-oxo-2-[(1H-[1,2,3]triazol-4-ylmethyl)-carbamoyl]-1,2,4,5,6,7-hexahydro-azepino[3,2,1-hi]indol-5-ylcarbamoyl}-butyl)-amino]-propionic acid [Scheme 55, Step-243]: To a stirred solution of Compound 213 (120 mg, 0.18 mmol) in DCM (2.00 mL), was added TFA (0.50 mL) under inert atmosphere at room temperature and stirred for 2 h. On completion of the reaction (confirmed by LC-MS), crude reaction mixture was submitted for reverse phase prep HPLC purification to afford faster eluting isomer [Isomer 1] (7.00 mg, 6.36%) of C230 as white solid. Mass [ESI]: m/z 615.65 [M⁺+1].

The above procedures describe synthesis for exemplary compounds wherein X is CH₂. The compounds within the scope of this disclosure wherein X is O, S or Sulfoxide, or NR can be prepared in a similar manner by following the experimental procedures described in PCT/US2019/017802.

### Example 2: Evaluation of Compounds in Microsomal Stability Assay

Certain compounds of this disclosure were tested in the microsomal stability assay provided above. Results are shown in Table 6 below.

TABLE 6
Compounds Stability in Human Liver Microsomes

| | | Human Liver |
|---|---|---|
| | | Microsomes |
| | | % remaining @30 |
| Compound | Name | min |
| C155 | (2S,5S)-5-((25,3S)-2-{[2-(2-Fluoro-ethoxy)-acetyl]-methyl-amino}- | 66.3 |
| | 3-methyl-pentanoylamino)-4-oxo-1,2,4,5,6,7-hexahydro-azepino[3,2,1- | |
| | hi]indole-2-carboxylic acid (1H[1,2,3]triazol-4-ylmethyl)-amide | |
| C178 | (2S,5S)-5((2S,3S)-2- {(2-Benzooxazol-2-yl-acetyl)-[2-(2-fluoro-ethoxy)- | 0.8 |
| | ethyll-aminol}-3-methyl-pentanoylamino)-4-oxo-1,2,4,5,6,7- | |
| | hexahydro-azepino[3,2,1-hi] indole-2-carboxylic | |
| | acid (1H[-1,2,3]triazol-4-ylmethyl)-amide | |
| C191 | N-Methyl-N-((1S,2S)-2-methyl-1-{(2S,5S)-4-oxo- | 83.6 |
| | 2-[(1H-[1,2,3]triazol-4-ylmethyl]- | |
| | carbamoyll-1,2,4,5,6,7-hexahydro-azepino[3,2,1-hi]indol- | |
| | 5-ylcarbamoyl}-buty1)-malonamide | |
| C192 | N,N'-Dimethyl-N-((lS,2S)-2-methy1-1-{(2S,5S)-4- | 72.8 |
| | oxo-2-[(1H-[1,2,3]triazol-4-ylmethyl)-carbamoyl]- | |
| | 1,2,4,5,6,7-hexahydro-azepino[3,2, | |
| | 1-hi]indol-5-ylcarbamoyl}-buty1)-malonamide | |
| C198 | (2S,5S)-5-{(2S,3S)-2-{(2-Imidazol-1-yl-acetyl)- | 55.4 |
| | methyl-amino]-3-methyl-pentanoylamino}-4-oxo- | |
| | 1,2,4,5,6,7-hexahydro-azepinop[3,2,1-hi]indole-2- | |
| | carboxylic acid (1H-[1,2,3]triazol-4-ylmethyl)-amide | |
| C214 | N-Methyl-N-((1S,2S)-2-methyl-1-{(1S,8S)-9-oxo- | 100.0 |
| | 1-[(1H-[1,2,3]triazol-4-ylmethyl)- | |
| | carbamoyl]-1,2,8,9-tetrahydro-7H-6-oxa-9a-aza- | |
| | benzo[cd]azulen-8-ylcarbamoyl}-butyl)-malonamic acid | |
| C230 | 3-[[2-(2-Fluoro-ethoxy)-acety1]-((1S,2S)-2-methyl-1- {(2S,5S)-4-oxo-2- | 98.2 |
| | [(1H-1,2,3]triazol-4-ylmethyl)-carbamoy1]-1,2,4,5,6,7-hexahydro- | |
| | azepino[3,2,1-hi]indol-5-ylcarbamoyl}-butyl)-amino]-propionic acid | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A compound of Formula I: 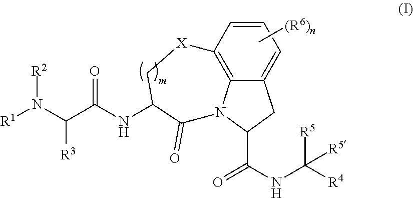
or a pharmaceutically acceptable salt thereof, or a stereoisomer or tautomer thereof, wherein:
R¹ is C₁₋₆ alkyl, C₁₋₆ heteroalkyl, C₃₋₈ cycloalkyl, C₂₋₈ heterocycloalkyl, —C(O)C₁₋₆ alkyl, —C(O)C₁₋₆ heteroalkyl, —C(O)C₃₋₈ cycloalkyl, —C(O)C₂₋₈ heterocycloalkyl, —C(O)aryl, or —C(O)heteroaryl;
R² is C₁₋₆ alkyl, C₁₋₆ alkoxy, or C₁₋₆ heteroalkyl;
R³ is hydrogen, C₁₋₆ alkyl, or C₃₋₈ cycloalkyl;
R⁴ is —C(O)OR, C₁₋₆ alkoxy, C₁₋₆ haloalkyl, —B(OR′)₂, —PO(OR″)₂, or heteroaryl;
each of R⁵ and R^{5′} is independently selected from the group consisting of hydrogen, halo, COOH, hydroxy, C₁₋₆ alkyl, C₁₋₆ alkoxy, and C₁₋₆ haloalkyl;
R⁶ is hydrogen, halo, OH, NRR′, —C(O)OR, —C(O)NRR′, C₁₋₆ alkyl, C₁₋₆ alkoxy, aryl, or heteroaryl;
X is CH₂, O, S, SO₂, or NR;
m is 0, 1, or 2; and
n is 1, 2, or 3,
wherein each of the C₁₋₆ alkyl, C₁₋₆ alkoxy, C₁₋₆ haloalkyl, C₁₋₆ heteroalkyl, C₃₋₈ cycloalkyl, C₂₋₈ heterocycloalkyl, aryl, and heteroaryl is optionally substituted with one or more moieties selected from the group consisting of oxo, halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′;
wherein each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
wherein at least one of R¹, R², R³, and R⁶ optionally comprises an imaging agent or a radioisotope.

2. The compound of claim 1, wherein X is CH₂.

3. The compound of claim 1, wherein R¹ is C₁₋₆ alkyl, C₁₋₆ heteroalkyl, —C(O)C₁₋₆ alkyl, or —C(O)C₁₋₆ heteroalkyl.

4. The compound of claim 1, wherein R² is C₁₋₆ alkyl or C₁₋₆ heteroalkyl.

5. The compound of claim 1, wherein R³ is C₁₋₆ alkyl.

6. The compound of claim 1, wherein R⁴ is heteroaryl and each of R⁵ and R^{5′} is hydrogen, wherein the heteroaryl is optionally substituted with 1, 2, or 3 substituents independently selected from the group consisting of oxo, halo, hydroxy, C₁₋₆ alkyl, and C₁₋₆ haloalkyl.

7. The compound of claim 2, wherein R¹ is C₁₋₆ alkyl, C₁₋₆ heteroalkyl, —C(O)C₁₋₆ alkyl, or —C(O)C₁₋₆heteroalkyl.

8. The compound of claim 2, wherein R² is C₁₋₆ alkyl or C₁₋₆ heteroalkyl.

9. The compound of claim 2, wherein R³ is C₁₋₆ alkyl.

10. The compound of claim 2, wherein R⁴ is heteroaryl and each of R⁵ and R^{5′} is hydrogen, wherein the heteroaryl is optionally substituted with 1, 2, or 3 substituents independently selected from the group consisting of oxo, halo, hydroxy, C₁₋₆ alkyl, and C₁₋₆ haloalkyl.

11. The compound of claim 2, wherein R¹ is C₁₋₆ alkyl, C₁₋₆ heteroalkyl, —C(O)C₁₋₆ alkyl, or —C(O)C₁₋₆heteroalkyl; and R² is C₁₋₆ alkyl or C₁₋₆ heteroalkyl.

12. The compound of claim 2, wherein R³ is C₁₋₆ alkyl, R⁴ is heteroaryl, and each of R⁵ and R^{5′} is hydrogen.

13. The compound of claim 2, wherein R¹ is C₁₋₆ alkyl, C₁₋₆ heteroalkyl, —C(O)C₁₋₆ alkyl, or —C(O)C₁₋₆heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen.

14. The compound of claim 13, wherein R³ is butyl and R⁴ is unsubstituted triazole.

15. The compound of claim 14, wherein R³ is 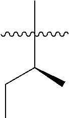 [SMILES: *[C@@H](C)CC]
and R⁴ is

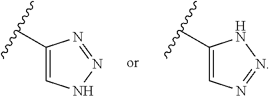 [SMILES: *c1c[nH]nn1 ; "or" ; *c1cnn[nH]1]

16. The compound of claim 1, wherein R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen.

17. The compound of claim 16, wherein R³ is 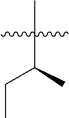 [SMILES: *[C@@H](C)CC]
and R⁴ is

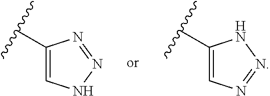 [SMILES: *c1c[nH]nn1 ; "or" ; *c1cnn[nH]1]

18. The compound of claim 17, wherein R¹ is —C(O)C₁₋₆ alkyl or —C(O)C₁₋₆ heteroalkyl.

19. The compound of claim 18, wherein R¹ is —C(O)C₁₋₆ alkyl substituted with aryl or heteroaryl.

20. The compound of claim 18, wherein R¹ is —C(O)C₁₋₆ heteroalkyl substituted with F.

21. The compound of claim 1, wherein X is O.

22. The compound of claim 21, wherein R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen.

23. The compound of claim 22, wherein R³ is butyl and R⁴ is unsubstituted triazole.

24. The compound of claim 23, wherein R³ is 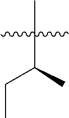 [SMILES: *[C@@H](C)CC]
and R⁴ is

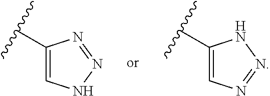 [SMILES: *c1c[nH]nn1 ; "or" ; *c1cnn[nH]1]

25. The compound of claim 24, wherein R¹ is —C(O)C₁₋₆ alkyl or —C(O)C₁₋₆ heteroalkyl.

26. The compound of claim 25, wherein R¹ is —C(O)C₁₋₆ alkyl substituted with aryl or heteroaryl.

27. The compound of claim 25, wherein R¹ is —C(O)C₁₋₆ heteroalkyl substituted with F.

28. The compound of claim 1, wherein X is NR.

29. The compound of claim 28, wherein R¹ is C₁₋₆ alkyl, C₁₋₆ heteroalkyl, —C(O)C₁₋₆ alkyl, or —C(O)C₁₋₆ heteroalkyl; R² is C₁₋₆ alkyl or C₁₋₆ heteroalkyl; R³ is C₁₋₆ alkyl; R⁴ is heteroaryl; and each of R⁵ and R^{5′} is hydrogen.

30. The compound of claim 29, wherein R³ is butyl and R⁴ is unsubstituted triazole.

31. The compound of claim 30, wherein R³ is 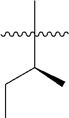 [SMILES: *[C@@H](C)CC]
and R⁴ is

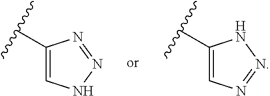 [SMILES: *c1c[nH]nn1 ; "or" ; *c1cnn[nH]1]

32. The compound of claim 31, wherein R¹ is —C(O)C₁₋₆ alkyl or —C(O)C₁₋₆ heteroalkyl.

33. The compound of claim 32, wherein R¹ is —C(O)C₁₋₆ alkyl substituted with aryl or heteroaryl.

34. The compound of claim 32, wherein R¹ is —C(O)C₁₋₆ heteroalkyl substituted with F.

35. The compound of claim 1, wherein the compound is one of the following compounds:

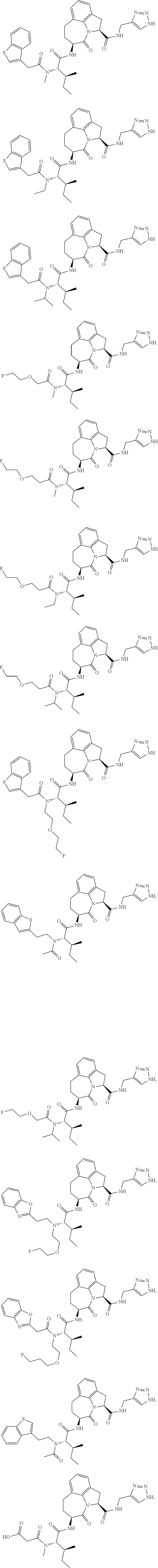

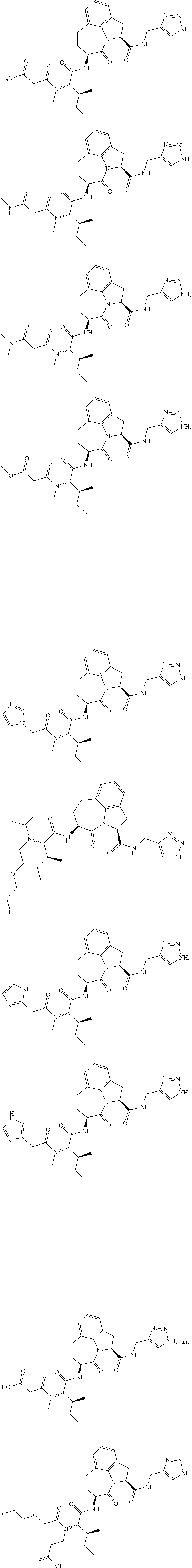

36. The compound of claim 1, wherein the compound is one of the following compounds: 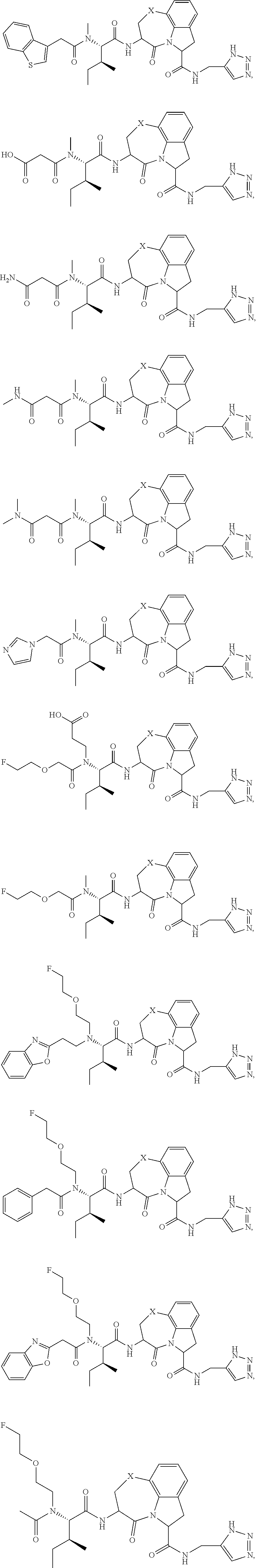
wherein X is CH₂, O, S, SO₂, or NR, in which R is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl.

37. The compound of claim 1, wherein at least one of R¹, R², R³, and R⁶ comprises an imaging agent.

38. The compound of claim 37, wherein the said imaging agent is selected from a group consisting of a PET imaging agent, a SPECT imaging agent, and a computed tomography imaging agent.

39. The compound of claim 37, wherein the said imaging agent is selected from a group consisting of a paramagnetic ion, an x-ray imaging agent, a fluorophore and a radioisotope.

40. The compound of claim 1, wherein at least one of R¹, R², R³, and R⁶ contains a radioisotope.

41. The compound of claim 40, wherein the said radioisotope is ¹⁸F.

42. The compound of claim 1, wherein at least one of R¹, R², R³, and R⁶ contains a PET imaging agent.

43. The compound of claim 42, wherein the said PET imaging agent comprises ¹⁸F.

44. The compound of claim 1, which is a compound of the following formula: 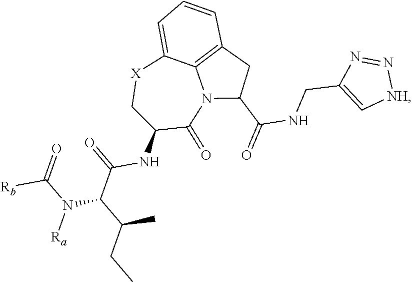
wherein X is CH₂, O, S, SO₂, or NR, in which R is H, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl,
Rₐ is C₁₋₆ alkyl or C₁₋₆ heteroalkyl in which the C₁₋₆ alkyl or C₁₋₆ heteroalkyl is optionally substituted with a halo, heteroaryl, or C(O)ORc,
R_{b} is C₁₋₆ alkyl or C₁₋₆ heteroalkyl in which the C₁₋₆ alkyl or C₁₋₆ heteroalkyl is optionally substituted with a halo, heteroaryl, —C(O)OR_{c}, or —C(O)NR_{c}R_{d},
R_{c} and R_{d}, independently, is H or C₁₋₆ alkyl,
wherein at least one of the moieties corresponding to R¹, R², R³, and R⁶ of Formula I optionally comprises an imaging agent or a radioisotope.

45. The compound of claim 44, wherein the moiety

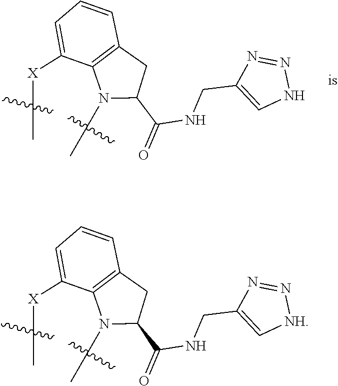

46. The compound of claim 44, wherein Rₐ is C₁₋₆ alkyl.

47. The compound of claim 44, wherein Rₐ is C₁₋₆ alkyl substituted with —C(O)OR_{c}.

48. The compound of claim 44, wherein Rₐ is C₁₋₆ alkyl substituted with a heteroaryl.

49. The compound of claim 48, wherein Rₐ is C₁₋₆ alkyl substituted with a benzothiophenyl.

50. The compound of claim 44, wherein Rₐ is C₁₋₆ heteroalkyl substituted with F.

51. The compound of claim 44, wherein R_{b} is C₁₋₆ alkyl.

52. The compound of claim 44, wherein R_{b} is C₁₋₆ alkyl substituted with —C(O)OR_{c} or —C(O)NR_{c}R_{d}.

53. The compound of claim 44, wherein R_{b} is C₁₋₆ alkyl substituted with a heteroaryl.

54. The compound of claim 44, wherein R_{b} is C₁₋₆ alkyl substituted with a heteroaryl selected from imidazolyl, benzothiophenyl, and benzooxazolyl,

55. The compound of claim 44, wherein R_{b} is C₁₋₆ heteroalkyl substituted with F.

56. A pharmaceutical composition comprising a compound of claim 1 and a pharmaceutically acceptable carrier.

57. A method of treating an immunoregulatory abnormality in a subject in need thereof, the method comprising administering to said patient a compound of claim 1 in an amount effective for treating said immunoregulatory abnormality.
